(19) United States
(12) Patent Application Publication
(10) Pub. No.: US 2021/0054323 A1
(43) Pub. Date: Feb. 25, 2021

(54) INNERVATED INTESTINE ON CHIP

(71) Applicant: EMULATE, Inc., Boston, MA (US)

(72) Inventors: Catherine Karalis, Brookline, MA (US); losif Pediaditakis, Boston, MA (US); Athanasia Apostolou, Brookline, MA (US)

(21) Appl. No.: 17/017,029

(22) Filed: Sep. 10, 2020

Related U.S. Application Data
(63) Continuation of application No. PCT/US19/22082, filed on Mar. 13, 2019.
(60) Provisional application No. 62/756,919, filed on Nov. 7, 2018.
(60) Provisional application No. 62/643,016, filed on Mar. 14, 2018.

(51) Int. Cl. C12M 3/06; B01L 3/00; G01N 33/50; C12M 1/12

(52) U.S. Cl. CPC C12M 23/16; B01L 3/502753; G01N 33/5082; G01N 2800/06; B01L 2200/0647; B01L 2300/0816; B01L 2300/0887; C12M 25/02

(57) ABSTRACT

The present invention relates to a combination of cell culture systems and microfluidic fluidic systems for use in providing a human innervated Intestine On-Chip. More specifically, in some embodiments, a microfluidic chip containing intestinal epithelial cells co-cultured with intestinal endothelial cells or intestinal muscle cells, or both, in the presence of induced neural crest cells may find use in providing an innervated Intestine-On-Chip. In some embodiments, an innervated Intestine On-Chip may be used for identifying (testing) therapeutic compounds for use in treating gastrointestinal disorders or diseases.

### FIELD OF THE DISCLOSURE

The present invention relates to a combination of cell culture systems and microfluidic fluidic systems for use in providing a human innervated Intestine On-Chip. More specifically, in some embodiments, a microfluidic chip containing intestinal epithelial cells co-cultured with intestinal endothelial cells or intestinal muscle cells, or both, in the presence of induced neural crest cells may find use in providing an innervated Intestine-On-Chip. In some embodiments, an innervated Intestine On-Chip may be used for identifying (testing) therapeutic compounds for use in treating gastrointestinal disorders or diseases.

### BACKGROUND

Healthy intestinal function is often disrupted by human conditions including disorders and diseases.

There is a need for a better platform to test therapeutic compounds for treating gastrointestinal diseases.

### SUMMARY OF THE INVENTION

The present invention relates to a combination of cell culture systems and microfluidic fluidic systems for use in providing a human innervated Intestine On-Chip. More specifically, in some embodiments, a microfluidic chip containing intestinal epithelial cells co-cultured with intestinal endothelial cells or intestinal muscle cells, or both, in the presence of induced neural crest cells may find use in providing an innervated Intestine-On-Chip. In some embodiments, an innervated Intestine On-Chip may be used for identifying (testing) therapeutic compounds for use in treating gastrointestinal disorders or diseases.

The present invention, in one embodiment, contemplates a method of culturing cells, comprising: a) providing a microfluidic device comprising a membrane, said membrane comprising a top side and a bottom side; b) seeding neurons on said top side and non-neuronal cells on said bottom side so as to create seeded cells; c) exposing at least some of said seeded cells to a flow of culture fluid for a period of time; and d) culturing said seeded cells under conditions such that contact is made between some of said neurons with some of said non-neuronal cells. The neurons need not contact said top side (e.g. they can rest on a gel which is in contact with said top side). It is not intended that the flow be the same for all cells. For example, neurons could be exposed to a low shear flow (or even a static or near static flow), while the non-neuronal cells (e.g. intestinal cells) could be exposed to flow (or even a high shear flow). It is also not intended that this embodiment be limited to just two cell types. For example, in one embodiment, said seeded cells further comprise endothelial cells (and these cells could be in the same compartment or side of the membrane, or in a different compartment or channel). It is not intended that the culture fluid be limited to culture media. For example, in one embodiment, said culture fluid comprises blood or blood components. It is not intended that this embodiment be limited to the manner by which contact is made. For example, in one embodiment, said neurons comprise neuronal terminal bulbs and said contact is made between said bulbs and said non-neuronal cells. In one embodiment, the cells are cultured under conditions such that at least some of said neurons extend processes across said membrane. In one embodiment, the cells are cultured under conditions such that contact is made by at least partial transmigration of said membrane by said neuron (or portion thereof) or neurons. It is not intended that the present invention be limited by the type of non-neuronal cell. In one embodiment, said non-neuronal cells are intestinal cells. In one embodiment, said intestinal cells are Human Colonic Microvascular Epithelial cells. In one embodiment, said intestinal cells are Human Intestinal Smooth Muscle cells. In one embodiment, said intestinal cells are Human Colonic Microvascular Epithelial cells. In one embodiment, said non-neuronal cells are selected from the group consisting of lung epithelial cells and skin epithelial cells. In one embodiment, said neurons are sensory neurons (including but not limited to the sensory neurons derived from Neural Crest cells as described herein). In one embodiment, said sensory neurons are not derived from Neural Crest cells. In one embodiment, the microfluidic device comprises a first channel on the topside of the membrane and a second channel on the bottom side. In one embodiment, the microfluidic device comprises a third channel (e.g. for a third cell type).

The present invention provides a method of culturing cells, comprising: a) providing a microfluidic device comprising a membrane, said membrane comprising a top side and a bottom side; b) seeding sensory neurons on said top side and non-neuronal cells on said bottom side so as to create seeded cells; c) exposing at least some of said seeded cells to a flow of culture fluid for a period of time; d) culturing said seeded cells under conditions such that contact is made between some of said neurons with some of said non-neuronal cells and e) measuring a function of said sensory neurons. In one embodiment, said non-neuronal cells are endothelial cells. In one embodiment, said function is barrier function. In one embodiment, said function is intracellular calcium ion fluctuations. In one embodiment, said function is Substance P secretion. In one embodiment, said method further comprises exposing said sensory neurons to a pain sensitizer chemical for increasing said function. In one embodiment, said pain sensitizer chemical is Capsaicin (trans-8-methyl-N-vanillyl-6-nonenamide). In one embodiment, said method further comprises exposing said endothelial cells to one or more inflammatory mediators. In one embodiment, said inflammatory mediators are selected from the group consisting of Prostaglandin E2, bradykinin, serotonin and histamine. In one embodiment, said method further comprises exposing said seeded cells to a drug for reducing said sensory function.

### BRIEF DESCRIPTION OF THE DRAWINGS

Exemplary embodiments are illustrated in referenced figures. It is intended that the embodiments and figures disclosed herein are to be considered illustrative rather than restrictive.

The file of this patent contains at least one drawing executed in color. Copies of this patent with color drawings will be provided by the Patent and Trademark Office upon request and payment of the necessary fee.

FIG. 1A illustrates a perspective view of a microfluidic device with microfluidic channels in accordance with an embodiment.

FIG. 1B illustrates an exploded view of the device in accordance with an embodiment, showing a microfluidic channel in a top piece and a microfluidic channel in a bottom piece, separated by a membrane.

FIG. 2A-B illustrates one embodiment of a microfluidic intestine-on-chip. FIG. 2A. In one embodiment a microfluidic intestine-on-chip is comprised of at least two micro-channels (blue and pink channels) separated by a porous flexible-membrane. The material is functionalized with specific extracellular matrix and cells are seeded into the different channels. In one embodiment of a fluidic Gut-Chip model human endothelial cells, such as HIMECs, are seeded in the bottom compartment, i.e. lower channel (pink) and human colorectal epithelial cells in the top compartment, i.e. upper channel (blue) to emulate the basic functioning unit of an organ, e.g. gut. In some embodiments, immune cells are added, e.g. isolated resident intestinal immune cells, PBMCs, T-cell populations, etc. In some embodiments, sensory neurons are added, such as iNeruons as described herein. Vacuum pressure can (optionally) be applied to the side channels (gray boxes) to mechanically stretch the membrane. Fluids can be continuously pumped through the channels to mimic shear forces, bring in nutrients, and wash away waste. In another embodiment, a more advanced Gut-Chip, resident immune cells and sensory neurons are incorporated in a gel in between the intestinal epithelial cells and the chip membrane.

FIG. 2B is an exemplary SEM image showing human intestine cells forming villi-like structures inside an exemplary Intestine-Chip representing a human innervated Intestine-Chip (hiIC) incorporating intestinal epithelial cells, intestinal endothelial cells, and (in preferred embodiments) resident immune cells, sensory neurons, and a representative microbiome.

FIG. 3A shows exemplary spontaneous activity studied by means of calcium imaging, enabling the investigation of neuronal activity of single cells and the network as a whole. Fluo-4 AM (shown as green labeled cells), a calcium sensitive fluorescent dye, was loaded onto the cells, and intracellular calcium fluctuations were monitored over time (left to right, 0, 30 and 60 seconds) using high-speed imaging.

FIG. 3B shows exemplary sensory neurons stained on-chip with Tuj-1 (green) for β-Tubulin 3, a pan-neuronal marker, TRPV1 (red) marker for sensory neurons identity (nociceptors). A merged image is shown in the lower panel where overlapping staining is shown in yellow. An enlarged area of Tuj-1+ neurons (top panel) is shown to the right.

FIGS. 4A-D shows an exemplary illustration of Enteric Nervous System (ENS) development.

FIG. 4A shows an exemplary illustration of Enteric Nervous System (ENS) in vivo.

FIG. 4B shows an exemplary illustration of ENS ganglia.

FIG. 4C shows an exemplary illustration of cell development from cranial, vagal and trunk areas of the neural crest, where arrows point to examples of derived cell types.

FIG. 4D shows an exemplary fluorescent micrograph of Phox2B (paired mesoderm homeobox protein 2B) expression in a mouse fetal gut.

FIG. 5 shows an exemplary protocol for Neural Crest differentiation. Starting on Day 0, 20,000 MEF cells/cm2 were seeded in CM including 10 ng/ml bFGF and 10 μM Y-27632 as PO Day 0. Day 2, cells were incubated in CM including 10 ng/ml bFGF. Day 3, now P1 Day 0, cells were plated in KSR media including 10 μM SB-431542 and 500 nM LDN-193189. P1 Day 1 KSR media including 10 μM SB-431542 and 500 nM LDN-193189. P1 Day 2 KSR media including 10 μM SB-431542, 500 nM LDN-193189 and 3 uM CHIR 99012. P1 Day 3 KSR media including 10 μM SB-431542 and 3 uM CHIR 99012. P1 Day 4 75% KSR media, 25% N2, including 3 uM CHIR 99012. P1 Day 4 75% KSR media, 50% N2, including 3 uM CHIR 99012 and 1 uM RA. P1 Day 8 25% KSR media, 75% N2, including 3 uM CHIR 99012 and 1 uM RA. P1 Day 10 N2, including 3 uM CHIR 99012 and 1 uM RA. P1 Day 11 N2, including 10 ng/ml bFGF and 10 ng/ml EGF. Sorting for HNK1+/p75− cells.

FIG. 6A-E shows exemplary results of magnetic beads sorting for HNK1− populations and HNK1+ and p75− populations. Left to right panels show increasing magnification of images, 5×, 10× and 20×.

FIG. 6A shows exemplary results of magnetic beads sorting for HNK1 and p75: HNK1− upper row. HNK1+/p75− lower row.

FIG. 6B shows exemplary results of magnetic beads sorting for HNK1 and p75: HNK1+/p75+ cells as double positive cells corresponding to the pure naïve Neural Crest cells population. Lower row of panels showing enlarged areas of upper panels, ×40.

FIG. 6C shows exemplary results of magnetic beads sorting for HNK1 and p75: Undifferentiated (left column) Pld11 (right column): HNK1 (green)/p75 (red)/DAPI (blue). For right panels, the enlarged area below is from the white boxed above.

FIG. 6D shows exemplary results of magnetic beads sorting for HNK1 and p75: Undifferentiated (left column) Pld11 (right column): HNK1 (green)/PAX6 (red)/DAPI (blue). The lower panels show an enlarged area of the white-boxed area above.

FIG. 6E shows exemplary results of magnetic beads sorting for HNK1 and p75: Undifferentiated (left column) and Pld11 (right column): AP2 (green)/PAX6 (red)/DAPI (blue). The lower panels show an enlarged area of the white boxed area above.

FIG. 7A illustrates a perspective view of an organ mimic device in accordance with an embodiment that contains three parallel microchannels separated by two porous membranes.

FIG. 7B illustrates a perspective view of an organ mimic device in accordance with an embodiment.

FIG. 7C illustrates the device containing three channels as described in FIG. 7A.

FIG. 8A-B shows exemplary bright-field microscopic images of NCM460 monolayer morphology. Left to right panels show images on Day 1, Day 2, Day 5, and Day 8.

FIG. 8A shows exemplary detachment of monolayer areas due to over confluency by Day 8 of culture (incubation). Upper row shows iNC plus cHIMEC (double membrane). Lower row shows iNC (LSR).

FIG. 8B shows exemplary cells that are present on Day 8 (far right panels). Upper row shows NCM460 plus cHIMEC (double membrane). Lower row shows NCM460 (LSR).

FIG. 9A-B shows exemplary barrier function comparing iNC+cHIMEC (red), iNC (blue) and cHIMEC (grey).

FIG. 9A shows that exemplary barrier function increased over time using 3 kDA particles.

FIG. 9B shows that exemplary barrier function increased over time using 450 kDA particles.

FIG. 10A-B shows that exemplary immunofluorescence—iNC differentiation assessment.

FIG. 10A shows exemplary images of glial cells. Middle row shows S100B glial cells colored green. Lower row shows Hoechst stained nuclei colored blue. Upper row of composite images shows overlapping green and blue stained gilal cells. Left two columns show NCM460 (LSR). Right two columns show NCM460 plus cHIMEC (double membrane) results. 10× magnification.

FIG. 10B shows enlargement of boxes corresponding to numbers 1 and 2 in FIG. 8A.

FIG. 11A-B shows that exemplary bright field images comparing NCM460 monolayers on-chip to iNCs on-chip. Day 2 (upper row) and Day 8 (lower row). Left column, NCM460/SMCs. Middle column, NCM460/cHIMECs. Right column, NCM460. 10× magnification with enlarged areas in the lower right or upper left in some panels.

FIG. 11A shows exemplary bright field images of NCM460 monolayer morphology.

FIG. 11B shows exemplary bright field images of iNC morphology.

FIG. 12 illustrates induction of iNC differentiation in presence of different intestinal cell types: NCM460/SMCs; NCM460/cHIMECs; or NCM460 in combination with iPS derived neural crest cells (iNC) elongated image, Human colonic epithelial cell line NCM460 as a blue cube, Human colonic microvascular epithelial cells (cHIMECs) as a source of NGF (red rectangle), Human intestinal smooth muscle cells (SMCs) as a source of GDNF (grey areas). In one embodiment, 6 days of culture under flow. Flow rate: 30 ul/hr.

FIG. 13 shows that exemplary expression of neuronal lineage markers in the differentiating population of iNCs. NCM460/cHIMECs (upper row). NCM460/SMCs (middle row). Lower row NCM460. 40× magnification. From left to right columns: Hoechst stained nuclei colored blue. S100B glial cells colored green. TUJ1 neurons colored red. Composite images shows overlapping blue, green and red stained cells.

FIG. 14 shows that exemplary functional characterization of differentiating iNC using Ca 2+ (colored green) imaging. Left panel shows cHIMECs. Right panel shows smooth muscle cells (SMCs).

FIG. 15A shows an exemplary schematic of an exploded view of an open top microfluidic chip.

FIG. 15B shows an exemplary schematic of an open top microfluidic chip showing embodiments of cellular locations. One embodiment is a schematic of a partial open top chip demonstrating channels and open area in relation to compartments in the chip (left). One embodiment as a schematic of a partial open top chip additionally demonstrating cells in the compartments of the chip (right). Comparative epithelial compartments include intestinal epithelium (enteroid/colonoid derived) from 4 different intestinal segments: duodenum, jejunum, ileum and colon. Stromal compartment includes intestinal fibroblasts+/−immune cells. Vascular compartment includes but is not limited to intestinal microvascular endothelium from small intestine and/or large intestine.

FIG. 15C shows another exemplary schematic of an open top microfluidic chip showing embodiments of a stretchable open top chip device 3200.

FIG. 15D shows another exemplary schematic of a fluidic gut-on-a-Chip wherein a body has a central microchannel therein; and an at least partially porous and at least partially flexible porous membrane positioned within the central microchannel and along a plane. The membrane is configured to separate the central microchannel to form a first central microchannel having a lumen side and a second central microchannel having a blood side, wherein a first fluid is applied through the first central microchannel and a second fluid is applied through the second central microchannel. There is at least one operating channel (vacuum chambers) separated from the first and second central microchannels by a first microchannel wall. The membrane is mounted to the first microchannel wall, and when a pressure is applied to the operating channel (one or more vacuum chambers), it can cause the membrane to expand or contract along the plane within the first and the second central microchannels. In some embodiments, one side of the membrane, example lumen side, can be seeded with epithelial cells to mimic an epithelial layer while another side of the membrane, example blood side, can be seeded with microvascular endothelial cells to mimic capillary vessels.

FIGS. 15E and 15F schematically depicts relaxed and elongated cell layers, via a stretched membrane, in an exemplary gut On-Chip. See, WO2013086486, Integrated Human Organ-On-Chip Microphysiological Systems. Publication Date: Jun. 13, 2013, herein incorporated by reference in its entirety.

FIG. 16 shows one embodiment for microfluidic devices as contemplated herein that is configured for electrophysiological measurements (e.g., for example, patch clamp measurements using transepithelial electric resistance (TEER).

FIG. 17 shows one embodiment for microfluidic devices as contemplated herein that is configured for electrophysiological measurements (e.g., for example, patch clamp measurements using transepithelial electric resistance (TEER).

FIG. 18 shows one embodiment for microfluidic devices as contemplated herein that is configured for electrophysiological measurements (e.g., for example, patch clamp measurements using transepithelial electric resistance (TEER).

FIG. 19 shows one embodiment for microfluidic devices as contemplated herein that is configured for electrophysiological measurements (e.g., for example, patch clamp measurements using transepithelial electric resistance (TEER). See, for examples, WO2017143049, Improved Blood-Brain Barrier Endothelial Cells Derived From Pluripotent Stem Cells For Blood-Brain Barrier Models, Publication Date: Aug. 24, 2017.

FIG. 20A-B shows an illustration of an architecture of the human blood-brain barrier. The normal architecture of the Human Blood Brain Barrier (top left) is mimicked by culturing human endothelium on one side of a porous membrane, in combination with human neuronal cells, human astrocytes, human lymphocytes, human pericytes, etc., with embedded platinum electrodes within an Organ Chip schematically shown in FIG. 20B. See, for nonlimiting examples, US20160145554A1, Integrated human organ-on-chip microphysiological systems, herein incorporated by reference in its entirety.

FIG. 20B shows an exemplary fluidic device, as one nonlimiting example, 250 μm tall, 500 μm wide, having a platinum electrode on one side, e.g. 200 μm wide. See, for examples without a specific description of innervated tissues, WO2013086486, Integrated Human Organ-On-Chip Microphysiological Systems. Publication Date: Jun. 13, 2013, herein incorporated by reference in its entirety. See, for examples, WO2017143049, Improved Blood-Brain Barrier Endothelial Cells Derived From Pluripotent Stem Cells For Blood-Brain Barrier Models, Publication Date: Aug. 24, 2017.

FIG. 21A shows an exemplary Morphological Characterization of the Neuronal Channel as one embodiment of an innervated Brain-chip. Scanning confocal microscope immunofluorescent images show immunohistochemistry staining of hiPSC-derived neuronal cultures using specific markers for discriminating neurons from astrocytes and pericytes, showing neurons (MPA2+, green) in direct contact with astrocytes (GFAP+, pink) and pericytes (NG2+, red), after 10 days of co-culture. Blue represents Hoechst-stained nuclei.

FIG. 21B shows an exemplary innervated Brain-chip further comprising Microglia, demonstrating that Microglia cells in neuron-microglial co-culture maintain their characteristics (ramified, referring to the cell body of the ramified form of microglia remains in place while its branches are constantly moving) on chip. Neurons (TUJ1-green), Microglia (Iba1-pink), Hoechst stain (nuclei-blue). Ionized calcium binding adaptor molecule 1 (Iba1) refers to a biomarker for a brain microglia calcium-binding protein.

FIG. 22A-D shows an exemplary morphological characterization of endothelial cells on a Brain chip by immunohistochemistry staining, shown as scanning confocal microscope immunofluorescent images in FIGS. 22A, 22C and 22D and flow cytometry analysis of endothelial cells FIG. 22D. Bar=50 μm.

FIG. 22A shows an exemplary immunocytochemical analysis of hiPSC-derived brain endothelial cells cultured on-chip for 7 days labeled with CD31 (white), F-actin (white), ZO-1 (green), GLUT-1 (red), Occludin (red), Claudin-5 (blue), and JAM-A (greenish-yellow)(bar, 50 μm).

FIG. 22B shows an exemplary immunofluorescence micrographic view of the human brain microvascular endothelium cultured on-chip for 7 days demonstrating high levels of expression of ZO-1 across the entire endothelial cell monolayer (bar, 50 μm).

FIG. 22C shows an exemplary immunofluorescence micrographic side view of stacked images for the human Brain-Chip showing the lower chamber covered by a continuous monolayer of brain endothelial cells stained for Von Willebrand factor (vWF, Cyan), and a mixture of pericytes (NG2, red), astrocytes (GFAP, magenta), glutamatergic and GABAergic neurons (MAP2, green) on the surface of the porous membrane in the upper chamber of the same chip.

FIG. 22D shows an exemplary flow cytometry analysis demonstrating the expression of ZO-1, and GLUT-1.

FIG. 23A-C shows exemplary Real-time TEER measurements in and exemplary BBB-chip. FIG. 23A (left) showing a gradual increase in barrier function upon initiation of flow and then an abrupt drop after TNF-a challenge. The barrier function of the same BBB-chip measured by flowing a fluorescent molecule on one side of the chip and measuring how much of the molecules crosses the endothelial cells, FIG. 23B (middle). The resolution of this measurement is limited by the time it takes for a sufficient sample to flow through the chip. A model of an exemplary TEER-Chip design FIG. 23C (right) utilizing electrodes oriented in a manner that allows electrical resistance of the cells on the membrane to be measured in real-time.

FIG. 24A-B shows an exemplary immunofluorescence micrographic view and quantitation of a Glutamatergic/GABAergic ratio observed in the Brain-Chip that has an vivo relevance with the cerebral cortex.

FIG. 24A shows exemplary glutamatergic neurons identified using vesicular glutamate transporter 1 (VGLUT1) antibodies (red) and GABArgic neurons identified using vesicular GABA transporter (VGAT) antibodies (green). Overlapping neurons, yellow, identify glutamatergic and GABAergic presynaptic terminals. Bar=50 μm.

FIG. 24B shows exemplary graphical quantitation as a percentage of the neuronal population on-chip.

FIG. 25 shows an exemplary assessment of the permeability in several embodiments of a Brain-Chip, e.g. astrocytes and pericytes seeded with iPS derived 1) glutamatergic neurons; 2) GABArgic neurons or 3) with both glutamatergic neurons and GABArgic neurons. A known concentration of a fluorescent protein, Dextran, was added in the vascular channel media; then after a defined period of time, the fluorescent dextran was quantified in the neuronal channel via effluent.

FIG. 26A-B shows an exemplary creation of an inflammatory environment on a Brain-chip. The vascular channel was directly stimulated the vascular channel at two concentrations of TNF-α. Exposure to TNF-α for 16 hours induces changes in endothelial cell morphology, and significantly increases the paracellular permeability.

FIG. 26A shows exemplary morphological disruption of endothelial cell expression of ZO-1, control-left panel, TNF-α (100 ng/ml)—middle panel and TNF-α 1 ug/ml—right panel, viewed as immunofluorescent micrographs. Bar=50 μm.

FIG. 26B shows exemplary quantitative correlation of a loss in barrier function, indicated by the disrupted ZO-1 expression. The percent leakage of Dextran into the apical channel is significantly higher than the control for both levels of TNF-α treatment.

FIG. 27A-B shows exemplary TNF-α treatment of one embodiment of a Brain-Chip significantly increases the GFAP expression (i.e. by astrocytes) up to 24 hours after stimulation. TNF-α (100 ng/ml) or vehicle was perfused into the neuronal channel.

FIG. 27A shows exemplary immunofluorescent micrographs of the neuronal channel demonstrating an increase in GFAP expression (pink); MAP2 (green); and Hoechst nuclear stain (blue) for vehicle, left panel (control) vs. TNF-α treatment, right panel.

FIG. 27A shows an exemplary graphical quatification of optical intensity of GFAP staining showing TNF-α (100 ng/ml) significantly increases the GFAP expression up to 24 hours after stimulation.

FIG. 28A-C shows exemplary sensory neurons, i.e. Nociceptors, in an apical channel on-chip, maintaining their neuronal characteristics on-chip after 7 days in culture.

FIG. 28A shows a fluorescent image of the entire neuronal channel showing Tuj-1+ neurons (green) upper panel; TRPV1 neurons (red) middle panel; and merged images showing yellow co-staining of Nociceptors, lower panel.

FIG. 28B shows exemplary Nociceptors as yellow co-stained Tuj-1+/TRPV1+ neurons, left panel, Tuj-1+ (green) upper right; TRPV1+ (red) lower right. Scale bars=50 μm.

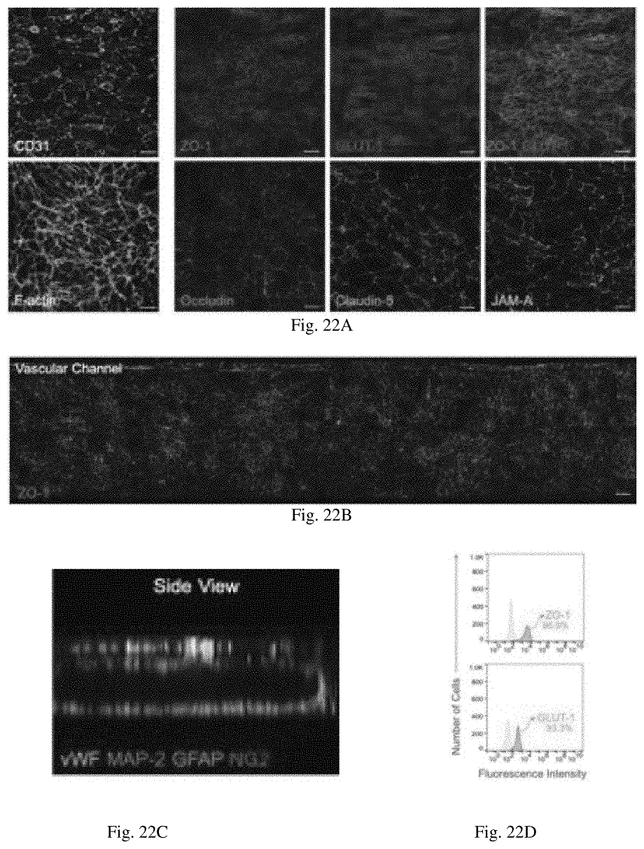

FIG. 22C shows exemplary Nociceptors stained with Fluo4-AM after irritation by Capsaicin (1 μM), see circled activated nodes.

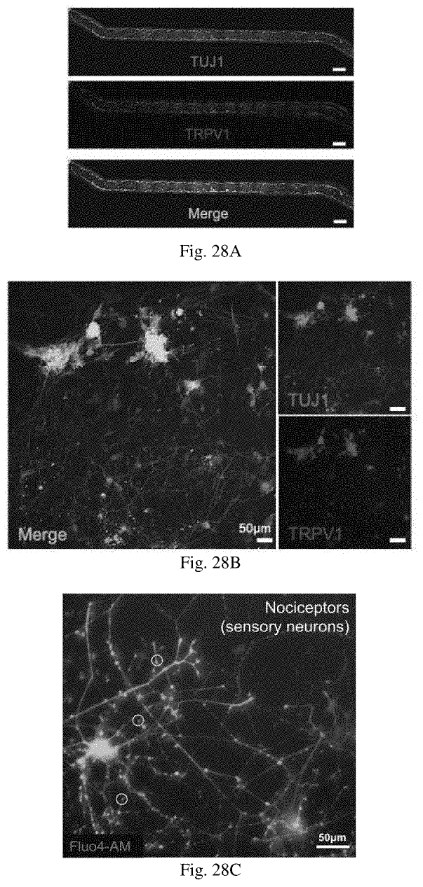

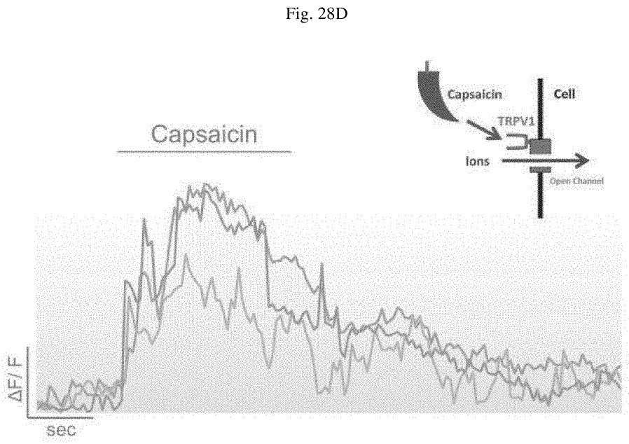

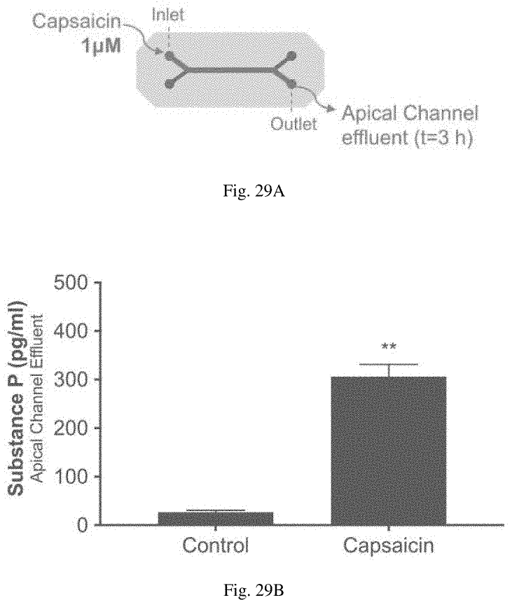

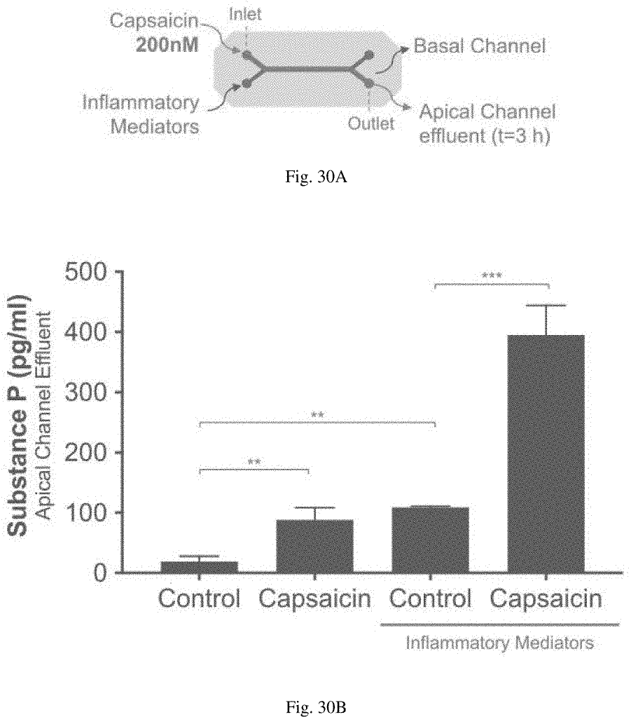

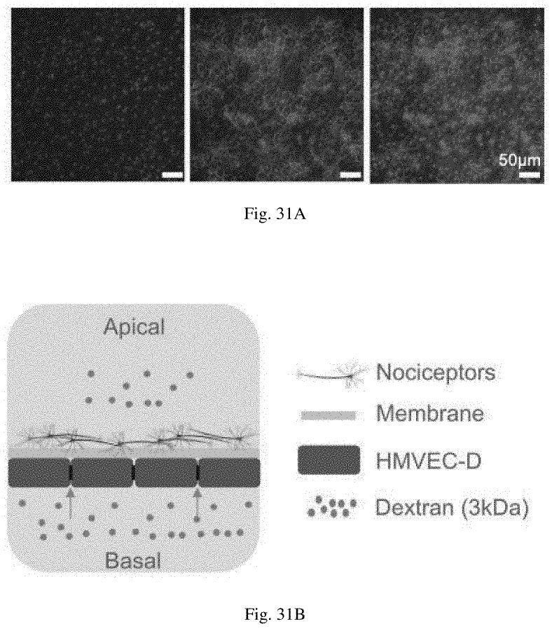

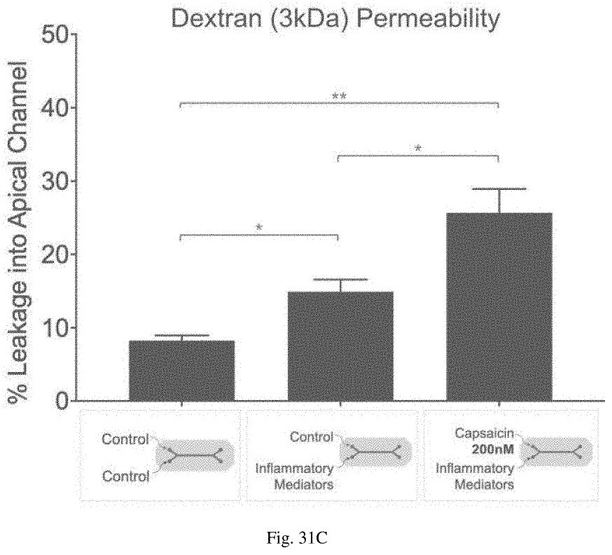

FIG. 28D shows an exemplary graph of Nociceptors responding to capsaicin, as shown by live imaging of calcium, change in fluorescence (ΔF) compared to fluorescence before stimulation (F).

FIG. 29A-B shows an exemplary read-out of substance P produced by chronic stimulation of Nociceptors (sensory neurons) though perfusion of the neuronal channel with capsaicin.

FIG. 29A shows an exemplary schematic of a closed channel chip comprising nociceptors in the apical channel perfused with capsaicin (1 uM) through the inlet, under flow, where the effluent was collected from the outlet of the apical channel after 3 hours (t=3 h).

FIG. 29B shows an exemplary graph of Substance P (pg/ml) measured in the apical channel effluent, as described in FIG. 29A showing a significant increase in Substance P released after capsaicin.

FIG. 30A-B shows an exemplary read-out of substance P produced by chronic stimulation of Nociceptors (sensory neurons) though perfusion of the neuronal channel with capsaicin in the presence of inflammatory mediators simultaneously perfused through the basal channel.

FIG. 30A shows an exemplary schematic of a closed channel chip comprising nociceptors in the apical channel perfused with capsaicin (200 nM in media) through the inlet, under flow. At the same time, inflammatory mediators, i.e. Prostaglandin E2, bradykinin, serotonin and histamine (each 5 μM in media), were added to the inlet of the basal channel. The effluent was collected from the outlet of the apical channel after 3 hours (t=3 h).

FIG. 30B shows an exemplary graph of Substance P (pg/ml) measured in the apical channel effluent, as described in FIG. 29A showing a significant increase in Substance P released after capsaicin treatment in the presence of inflammatory mediators.

FIG. 31A-B shows an exemplary read-out of changes in vascular permeability produced by chronic stimulation of Nociceptors (sensory neurons) though perfusion of the neuronal channel with capsaicin in the presence of inflammatory mediators simultaneously perfused through the basal channel.

FIG. 31A shows exemplary florescent microscope images of endothelial cells in the basal channel. Left, Hoechst staining of nuclei (blue), middle, e-cadherin staining (pink) and right, merged florescent images showing individual cells (blue nuclei) surrounded by e-cadherin.

FIG. 31B shows an exemplary schematic of a vertical chip cross-section of a closed channel chip comprising Nociceptors in the apical channel separated from HMVEC-D by a membrane (yellow) where Dextran (3 kDa—blue dots) added to the basal channel leaked into the apical channel.

FIG. 31C shows an exemplary graphical comparison of leakage (percent) of 3 kDa Dextrin into the apical channel between controls, no treatment (left), inflammatory mediators (middle), demonstrating a significant amount of leakage after perfusion with capsaicin (200 nM in media) in the apical channel simultaneously with perfusion of inflammatory mediators, i.e. Prostaglandin E2, Bradykinin, serotonin and histamine (each 5 μM in media), added to the inlet of the basal channel, right, after 3 hours (t=3 h).

### DEFINITIONS

The term “microfluidic” as used herein relates to components where moving fluid is constrained in or directed through one or more channels wherein one or more dimensions are 1 mm or smaller (microscale). Microfluidic channels may be larger than microscale in one or more directions, though the channel(s) will be on the microscale in at least one direction. In some instances the geometry of a microfluidic channel may be configured to control the fluid flow rate through the channel (e.g. increase channel height to reduce shear). Microfluidic channels can be formed of various geometries to facilitate a wide range of flow rates through the channels.

“Channels” are pathways (whether straight, curved, single, multiple, in a network, etc.) through a medium (e.g., silicon) that allow for movement of liquids and gasses. Channels thus can connect other components, i.e., keep components “in communication” and more particularly, “in fluidic communication” and still more particularly, “in liquid communication.” Such components include, but are not limited to, liquid-intake ports and gas vents. Microchannels are channels with dimensions less than 1 millimeter and greater than 1 micron.

As used herein, the phrases “connected to,” “coupled to,” “in contact with” and “in communication with” refer to any form of interaction between two or more entities, including mechanical, electrical, magnetic, electromagnetic, fluidic, and thermal interaction. For example, in one embodiment, channels in a microfluidic device are in fluidic communication with cells and (optionally) a fluid reservoir. Two components may be coupled to each other even though they are not in direct contact with each other. For example, two components may be coupled to each other through an intermediate component (e.g. tubing or other conduit).

As used herein, the term “biopsy” refers to a sample of the tissue that is removed from a body.

As used herein, “Caco-2” or “Caco2” refer to a human epithlial intestinal cell line demonstrating a well-differentiated brush border on the apical surface with tight junctions between cells. Although this cell line was originally derived from a large intestine (colon) carcinoma, also called an epithelial colorectal adenocarcinoma, this cell line can express typical small-intestinal microvillus hydrolases and nutrient transporters, see. Meunier, et al., “The human intestinal epithelial cell line Caco-2; pharmacological and pharmacokinetic applications.” Cell Biol Toxicol. 11(3-4):187-94, 1995, abstract. Examples of Caco-2 cell lines include but are not limited to CRL-2102, American Type Culture Collection (Rockville, Md.); a BBE subclone of Caco-2 cells; etc.

### DESCRIPTION OF INVENTION

The present invention relates to a combination of cell culture systems and microfluidic fluidic systems for use in providing a human innervated Intestine-On-Chip. More specifically, in some embodiments, a microfluidic chip containing intestinal epithelial cells co-cultured with intestinal endothelial cells or intestinal muscle cells, or both, in the presence of induced neural crest cells may find use in providing an innervated Intestine-On-Chip. In some embodiments, an innervated Intestine-On-Chip may be used for identifying (testing) therapeutic compounds for use in treating gastrointestinal disorders or diseases.

Lack of a robust, well-characterized in vitro model for the human innervated-intestine significantly limits any potential progress that can be made in these areas, since current in vivo studies in humans are difficult to conduct and species differences in animal models result in poor predictors of human responses. Furthermore, in general there is a lack of in vitro models for human innervation of organs that closely mimic conditions in vivo.

There is also a lack of an in vitro model for a human innervated brain that closely mimics neuronal compositions of human cortical brain tissue. Thus, in some embodiments, innervated fluidic devices are Brain-Chips, e.g. human brain a blood brain barrier (BBB)-chips. Brain chips may comprise human primary astrocytes, primary pericytes, with a combination of iPS derived Glutamaterigic neurons and iPS derived GABAergic neurons at physiologic mixtures, cell lines of these cell types, etc., in the neuronal (apical) channel. Brain chips may further comprise endothelial cells in the basal channel, such as iPS derived brain endothelial cells. In some embodiments, Brain-chips are closed top chips with a porous membrane separating the apical and basal channels, optional vacuum channels, etc.

Therefore, the present invention relates to providing a human innervated microfluidic device. In some embodiments, innervated tissue, including parenchymal tissue, stromal tissue, etc., is innervated on chip for modeling associations with one or more conditions including but not limited to: inflammation, sensing pain, mobility disorders, etc. Innervation may be associated with or cause mobility disorders such as found in intestinal disorders or diseases. Such intestinal mobility disorders may involve inflammation, pain and spasmatic movements of the intestine.

### I. Types of Innervated Fluidic Devices.

The present invention relates to innervating fluidic devices with neurons. As used herein, a neuronal cell may comprise, depending upon the type of neuron, dendrites, a cell body (containing a nucleus), axon, myelin sheath (provided by a Schwaan cell and surrounds an axon with the exception of Nodes of Ranvier along the axon), axon terminals (synaptic bulbs). Thus, “innervation” refers to an association of at least one part of a neuronal cell with a nonneuronal cell.

It is not meant to limit the types of innervation. In fact, bodily organs are innervated in certain configurations having at least one or more types of functions. For example, innervation includes neurons capable of sensing pain and neurons having additional functions, and neurons not capable of sensing pain. In some embodiments, neuronal responses may be associated with inflammation. In some embodiments, neuronal responses may be associated with smooth muscle mobility disorders, such as intestinal (e.g. small intestine, large intestine, stomach, etc.), brain, lung, airway, skin, kidney, bladder etc. In some embodiments, fluidic devices are innervated having one or more of epithelial layers as parenchymal cells, etc. In some embodiments, fluidic devices have innervated specialized parenchymal cells, for example, lymph node on-chip having innervation (e.g. by noradrenergic nerve cells) of one or more cell types including but not limited to endothelial cells, simple cuboidal epithelium, smooth muscle cells, connective tissue, lamina propria (e.g. produced by reticular cells, fibroblasts, etc.), macrophages, etc.); spleen on-chip having innervation, including but not limited to one or more of noradrenergic (sympathetic) neurons (e.g. norepinephrine (NE) release); cholinergic (parasympathetic) neurons (catecholaminergic); neuron typically containing tyrosine hydroxylase (TH); neuron typically containing neurotransmitter nerve neuropeptide Y (NPY); neuron typically containing neurotransmitter vasoactive intestinal peptide (VIP); neurons typically containing calcitonin gene related peptide (CGRP); neurons typically containing substance P (SP), etc. In some embodiments, an innervated chip does not have parenchymal cells, such as a spleen on-chip, lymph-node on-chip, etc.

In some embodiments, innervation is innervation of muscle cells. In some embodiments, innervation is innervation of smooth muscle cells. In some embodiments, innervation is innervation of cardiac muscle cells.

In some embodiments, an innervated microfluidic chip comprising muscle cells is configured such that neurons may be electrically or chemically excited neurons without (predominantly) exciting the muscle cells. In some embodiments, such configuration comprises a membrane.

As further examples, SP+ and CGRP+ nerve terminals, as one example, in close proximity to marginal-zone-like macrophages and B cells; autonomic nervous system type neurons in driving and/or modulating splenic TNF production, mostly released by resident macrophages; splenic ‘inflammatory reflex’ refers to a complex response, containing both neural and non-neural elements. In its current concept, it includes autonomic neural circuits composed of noradrenergic (sympathetic) and cholinergic (parasympathetic) neurons as well as splenic T cells and macrophages turn influences macrophage polarization, T- and B-cell activation, and antibody production

Sympathetic and parasympathetic divisions of the autonomic nervous system with regard to regulating a splenic inflammatory response. Innervation by a neuron, where a neuron refers to the entire nerve cell, including a cell body, dendrite, axon and nerve terminal, may include innervation by one or more components of a neuron, a cell fiber, i.e. axon, bundle of axons, nerve terminal, with or without a myelin sheath, i.e. with or without Schwann cells, (e.g. having one or more cell types including but not limited to squamous epithelium, endothelium, connective tissue (e.g. produced by fibroblasts, etc.), macrophages, lymphocytes, including but not limited to lymphocytes producing acetylcholine (ACh) and expressing choline acetyltransferase (ChAT), and other types of white blood cells, red blood cells, etc.), blood-brain barrier on-chip, brain on-chip (e.g. having one or more cell types including but not limited to nervous tissue (nerve cells and glia), etc.

In some embodiments, innervated chips are contemplated to comprise microorganisms, including pathogenic microorganisms, such as pathogenic bacteria. In some embodiments, an innervated Intestine-Chip is contemplated for use in identifying therapeutic targets for infectious disorders of the intestine. In some embodiments, an innervated Intestine-Chip is contemplated for use in identifying candidate drugs for treating infectious disorders of the intestine. In some embodiments, an innervated Intestine-Chip will be used to assess the activation of the incorporated primary sensory neurons as an in vivo-relevant biological read out for the bacteria-mediated disruption of intestinal defense. In some embodiments, an innervated Intestine-Chip will be used as a model for studying host-pathogen interactions and investigating the probiotic function of commensal bacteria. In some embodiments, an innervated Intestine-Chip is contemplated for use in identifying therapeutic targets for autoimmune disorders of the intestine.

It is not meant to limit bacteria interactions to an innervated Intestine-Chip. In some embodiments, an innervated chip as described herein is used in combination with bacteria, e.g. pathogenic, nonpathogenic, commensal, etc.

In some embodiments, neurons interact with epithelial cells. However, it is not meant to limit the type of epithelial cells intended for innervation, such that epithelial cells include but are not limited to intestinal epithelial cells, such as small intestinal cells, large intestinal cells, colonic intestinal cells, etc.

The present invention relates to providing a human innervated Intestine-Chip (hiIC) comprised of colonic epithelial cells, colonic endothelial derived cells, and enteric sensory neurons derived from induced Pluripotent Stem cells (iPSc). It is not meant to limit the source of cell types used for providing enteric sensory neurons. Indeed several types of cells may be used for providing enteric sensory neurons, including but not limited to neural crest derived-iPS cells, fibroblast derived iPS cells, biopsy derived cells, etc. The present invention relates to providing a human innervated Intestine-Chip (hilC) comprised of colonic epithelial cells, colonic smooth muscle derived cells, and enteric sensory neurons derived from induced Pluripotent Stem Cells (iPSc).

The present invention relates to providing a human innervated Brain-chip, e.g. comprising human induced pluripotent stem cells (iPSC)-derived cortical neurons, i.e., Glutamatergic and GABAergic neurons, in addition to pericytes, astrocytes and microglial cells, in the neuronal channel co-cultured with brain endothelial cells, e.g., Human iPSC-derived brain microvascular endothelial cells, in the vascular channel.

In one embodiment, an innervated chip mimics a lymph node. Thus in some embodiments, neurons interacting with epithelial cells mimic organ-specific lymph nodes, such as areas of the intestine referred to as Peyer's Patches. In some embodiments, epithelial cells in vivo known as microfold cells line the side of the Peyer's patch facing the intestinal lumen, while the outer side of a Peyer's Patch contains many lymphoid cells and lymphatic vessels. Thus, in some embodiments, epithelial cells include microfold cells (or M cells).

In one embodiment, an Intestine-Chip (hilC) is innervated by neurons of the peripheral nervous system. In one embodiment, innervations of intestinal parenchymal cells are by sensory neurons. However, it is not meant to limit innervation to one the type of neuron. Thus, additionally embodiments of innervated fluidic devices may include motor neurons (which are those that specifically actuate muscle), neurons of the sympathetic nervous system, and neurons of the parasympathetic nervous system. The sympathetic and parasympathetic nervous systems include both afferent (sensory) and efferent (actuating) neurons.

In one embodiment, an innervated chip is a lung chip. In one embodiment, an innervated chip, such as a lung chip, an intestine chip, and the like, includes smooth muscle cells. In one embodiment, innervation is innervation of smooth muscle cells in a lung chip. In one embodiment, an innervated lung chip comprising smooth muscle cells is capable of mimicking muscle contractions such as is implicated in asthma, etc.

In one embodiment, an innervated chip comprises neurons co-cultured with endothelial cells. In one embodiment, an innervated chip comprises simultaneous seeding of neurons and endothelial cells. In one embodiment, an innervated chip comprises separate seeding of seeding of neurons and endothelial cells. In one embodiment, neurons are seeded on top of the membrane then endothelial cells are seeded on top of the neurons. In one embodiment, neurons are seeded in a gel on top of the membrane then endothelial cells are seeded on top of the gel containing neurons. In one embodiment, a fluidic chip comprises two membranes. In one embodiment, the two membranes are based upon a design disclosed in '861, thus providing three compartments: a top for compartment for endothelial cells, middle compartment for neurons, and a bottom compartment for epidermal cells. This embodiment, in part is based upon the observation that a) endothelial cells often improve maturation of the neurons, and may improve the organ-specific cells, and/or the junction/interaction of the two, b) endothelial cells typically improve the ability to capture inflammatory response, and c) there is desirable cell biology benefits associated with the interaction of neurons and endothelial cells, as one example semaphorins. See for example the sentence “Conversely, SEMA3A secreted by ischemic neurons prevents neovascularization of the injured area” in www.ncbi.nlm.nih.gov/pmc/articles/PMC4181631/. Nasarre, et al., “The emerging role of class-3 semaphorins and their neuropilin receptors in oncology.” OncoTargets and Therapy 7 1663-1687, 2014.

The present invention also relates to providing iPS derived Neural Crest Cells (iNC). Thus, in some embodiments, intestinal epithelial cells and/or intestinal endothelial derived cells are co-cultured with iPS derived Neural Crest Cells, e.g. (HNK1+/p75+ induced neural crest cells) for providing more mature neurons on-chips for use in intestinal chips.

In one embodiment, Human Colonic Microvascular Epithelial Cells (cHIMECs) provide a source of NGF for inducing differentiating iPS derived Neural Crest Cells on-chips.

In one embodiment, Human Intestinal Smooth Muscle Cells (SMCs) provide a source of GDNF for inducing differentiating iPS derived Neural Crest Cells on-chips.

The present invention also relates to providing a human innervated Intestine-Chip (hiIC) comprised of colonic epithelial cells, lamina propria derived resident immune cells, and enteric sensory neurons derived from induced Pluripotent Stem cells (iPSc). Thus, in one embodiment, iPS derived Neural Crest Cells (iNC) are added to microfluidic chips. In one embodiment, these cell types recapitulate a nondiseased intestinal environment. In one embodiment, these cell types recapitulate a normal intestinal barrier. In one embodiment, one or more of these cell types provide a model for intestinal diseases. In one embodiment, one or more of these cell types provide a model for intestinal diseases involving a neuronal component, either normal or abnormal of diseased.

In one embodiment, human sensory neurons are introduced into the chip and demonstrate survival and functionality by measuring neuron excitability. In one embodiment, analyze gene expression profiles and characterize the identity of all cells in the chip over the course of the culture.

As used herein, “muscle” may refer to any type of muscle, such as smooth, cardiac muscle, etc.

One embodiment of a microfluidic intestine-on-chip. In one embodiment, a microfluidic chip is comprised of at least two micro-channels (blue and pink channels) separated by a porous flexible-membrane. The material is functionalized with specific extracellular matrix and cells are seeded into the different channels. In our basic Gut-Chip model human endothelial cells are seeded in the bottom compartment (pink) and human colorectal epithelial cells in the top compartment (blue) to emulate the basic functioning unit of an organ. Vacuum pressure can be applied to the side channels (gray) to mechanically stretch the membrane. Fluids can be continuously pumped through the channels to mimic shear forces, bring in nutrients, and wash away waste. In the more advanced Gut-Chip, resident immune cells and sensory neurons are incorporated in a gel in between the intestinal epithelial cells and the chip membrane.

Spontaneous activity was studied by means of calcium imaging, enabling the investigation of neuronal activity of single cells and the network as a whole. Fluo-4 AM, a calcium sensitive fluorescent dye, was loaded onto the cells, and intracellular calcium fluctuations were monitored over time using high-speed imaging.

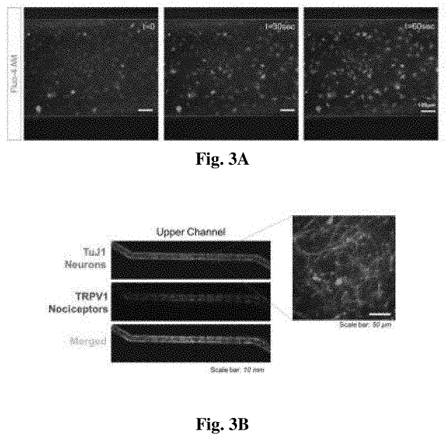

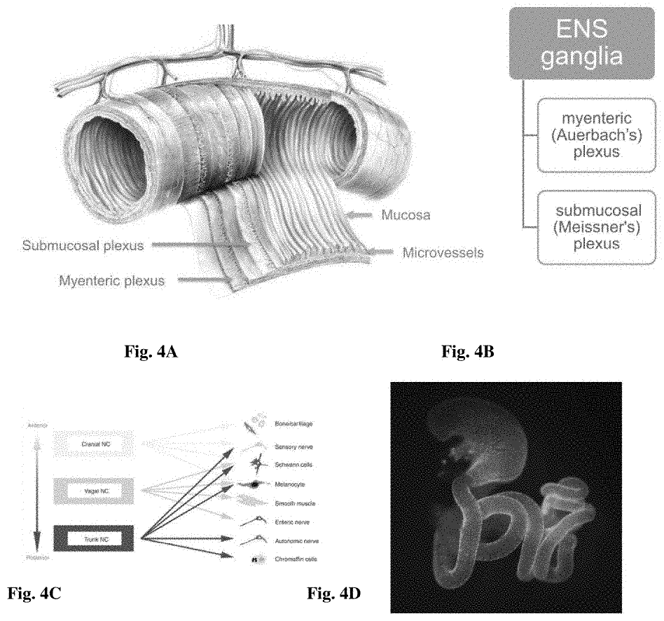

FIG. 3A shows exemplary spontaneous activity studied by means of calcium imaging, enabling the investigation of neuronal activity of single cells and the network as a whole. Fluo-4 AM (shown as green labeled cells), a calcium sensitive fluorescent dye, was loaded onto the cells, and intracellular calcium fluctuations were monitored over time (left to right, 0, 30 and 60 seconds) using high-speed imaging.

FIG. 3B shows exemplary sensory neurons stained on-chip with Tuj-1 (green) for β-Tubulin 3, a pan-neuronal marker, TRPV1 (red) marker for sensory neuron identity (Nociceptors). A merged image is shown in the lower panel where overlapping staining is shown in yellow. An enlarged area of Tuj-1+ neurons (top panel) is shown to the right.

In some embodiments, the iNC is being differentiated in the presence of intestinal epithelial cells. In some embodiments, the iNC is being differentiated in the presence of endothelial cells. In some embodiments, the iNC is being differentiated in the presence of both epithelial cells and endothelial cells.

In some embodiments, the iNC differentiates into a glial genotype. In some embodiments, the iNC differentiates into a glial phenotype. In some embodiments, the iNC differentiates into a neuronal genotype. In some embodiments, the iNC differentiates into a neuronal phenotype.

In some embodiments, as iNC differentiates, characteristics of epithelial layer homeostasis and function are measured.

A. Embodiments Including Gels.

In some embodiments, innervated fluidic devices comprise gels. In particular, because neurons in vivo naturally live in a matrix environment, a gel matrix is contemplated for use for providing a neuronal microenvironment, e.g. for neuronal dendrite extensions, cell bodies, etc. Thus, there are multiple locations within chips for including gels, such as gel layers, above, below or for adding so that neurons may extend into such gel layers. Thus in one embodiment, an entire neuronal cell, including cell bodies, extensions and dendrites are embedded within a gel layer. In one embodiment, at least one part of a neuronal cell is embedded in a gel layer, including but not limited to a neuronal cell growth into the gel layer. In one embodiment, at least one part of a neuronal cell is on top of a gel layer. In one embodiment, at least one part of a neuronal cell is below a gel layer. In one embodiment, at least one part of a neuronal cell is on the organ side of the model: e.g. Intestine-Chip with lamina propria and fibroblasts under the epidermal cells. In one embodiment, at least one part of a neuronal cell extends into a gel under muscle cells to provide a more suitable mechanical environment for muscle cell flexing, i.e. movement.

B. Types of Neurons.

In some embodiments, fluidic innervated devices are seeded with neuronal cells. In some embodiments, fluidic innervated devices are seeded with cells intended for differentiation into neuronal cells. It is not meant to limit the source of cell types used for providing sensory neurons. Indeed several types of cells may be used for providing sensory neurons, including but not limited to neural crest derived-iPSCs, neural tube-derived-iPSCs, fibroblast derived iPSCs, embryonic derived iPSCs, biopsy derived cells, patient specific cells, etc. Methods for dedifferentiating fibroblast or other cell types into iPSCs are well known. In some embodiments, innervation is by one or more of spinal motor neurons (spMNs) and dopaminergic neurons (DANs). In some embodiments, human iPSCs are directed to provide spinal motor neurons (spMNs). In some embodiments, human iPSCs are directed to provide dopaminergic neurons (DANs).

Thus, in one embodiment, innervation is by innervation of neural crest or neural crest-derived cells. In one embodiment, innervation is by cells not derived from neural crest cells. In one embodiment, innervation is by cells. In one embodiment, innervation is by sensory neurons. In one embodiment, innervation is innervation of muscle cells.

In some embodiments, fluidic innervated devices were seeded with iNC-derived cells.

Results of One Embodiment of a Fluidic Innervated Devices Seeded with iNC-Derived Cells:

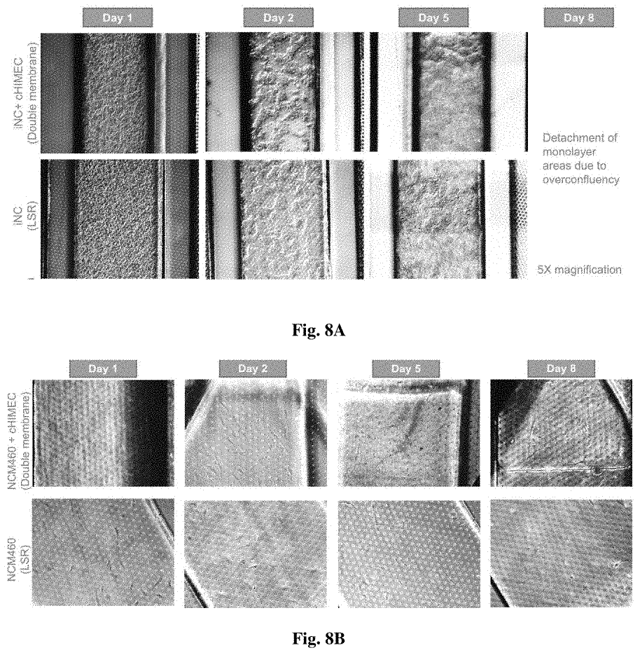

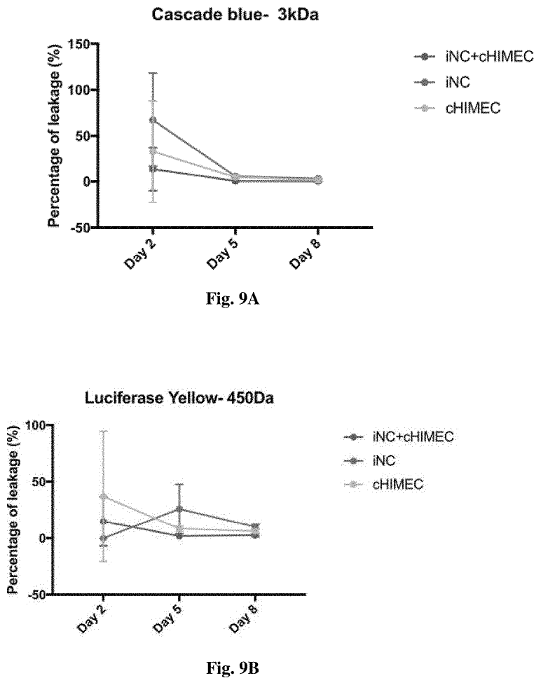

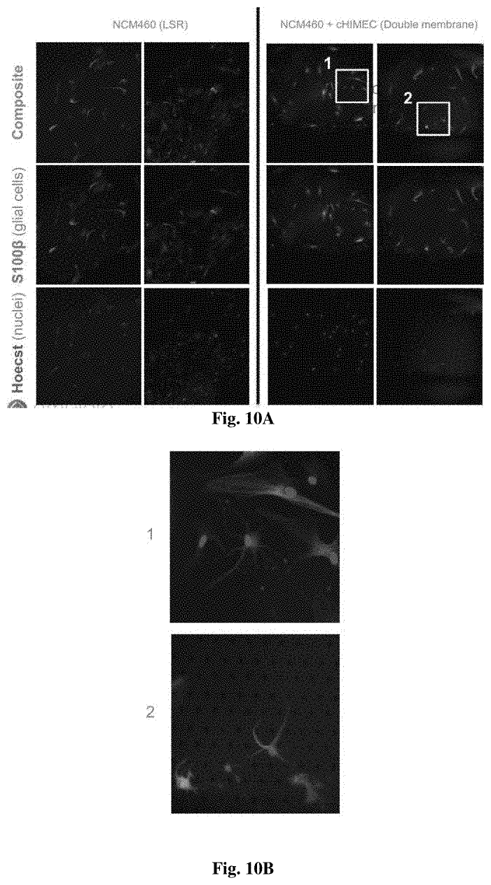

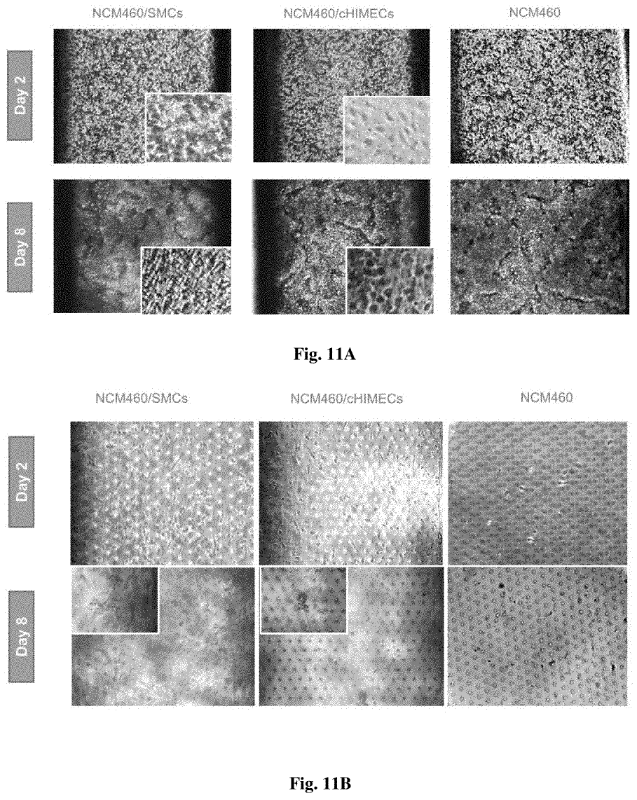

In some embodiments, the iNC cells were differentiated in the presence of cHIMECs. In some embodiments, the iNC cells were differentiated towards glial cells (glial fate) as indicated by the positive S1000 immuno staining. In some embodiments, there was little positive immunostaining for the paneuronal marker β-tubulin III (TUJ1) (nerve fate). In some embodiments, differentiation occurred over 7 days however the monolayers started lifting off of the chip surface by day 8. In some embodiments, on the day 8 the experiment was stopped because NCM460 cells were overconfluent and detached sporadically across the chip. See, FIG. 8A.

FIG. 8A-B shows exemplary bright-field microscopic images of NCM460 monolayer morphology. Left to right panels show images on Day 1, Day 2, Day 5, and Day 8.

FIG. 8A shows exemplary detachment of monolayer areas due to over confluency by Day 8 of culture (incubation). Upper row shows iNC plus cHIMEC (double membrane). Lower row shows iNC (LSR).

FIG. 8B shows exemplary cells that are present on Day 8 (far right panels). Upper row shows NCM460 plus cHIMEC (double membrane). Lower row shows NCM460 (LSR).

In some embodiments, the iNC cells were differentiated in the presence of cHIMECs, NCM460 cells and smooth muscle cells, showing cells growing on chips at Day 8. See, FIG. 11A-B. In some embodiments, the iNC are being differentiated in the presence of intestinal smooth muscle cells and/or endothelial cells.

FIG. 11A-B shows that exemplary bright field images comparing NCM460 monolayers on-chip to iNCs on-chip. Day 2 (upper row) and Day 8 (lower row). Left column, NCM460/SMCs. Middle column, NCM460/cHIMECs. Right column, NCM460. 10× magnification with enlarged areas in the lower right or upper left in some panels.

FIG. 11A shows exemplary bright field images of NCM460 monolayer morphology.

FIG. 11B shows exemplary bright field images of iNC morphology.

Induction of iNC differentiation in presence of different intestinal cell types: NCM460/SMCs; NCM460/cHIMECs; or NCM460. 6 days of culture under flow. Flow rate: 30 ul/hr.

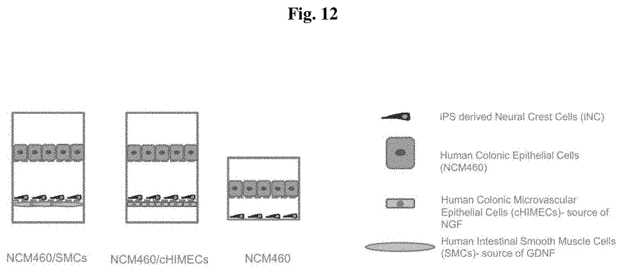

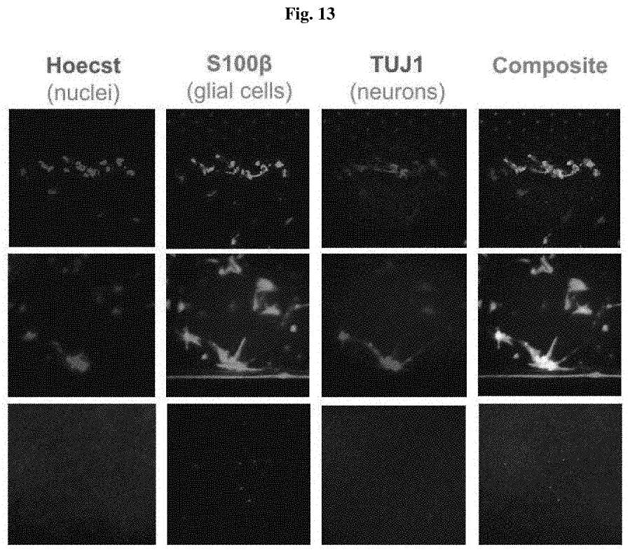

FIG. 12 illustrates induction of iNC differentiation in presence of different intestinal cell types: NCM460/SMCs; NCM460/cHIMECs; or NCM460 in combination with iPS derived neural crest cells (iNC) elongated image, Human colonic epithelial cell line NCM460 as a blue cube, Human colonic microvascular epithelial cells (cHIMECs) as a source of NGF (red rectangle), Human intestinal smooth muscle cells (SMCs) as a source of GDNF (grey areas). In one embodiment, 6 days of culture under flow. Flow rate: 30 ul/hr.

In some embodiments, the iNC cells were differentiated towards glial cells (glial fate) as indicated by the positive S1000 immuno staining. In some embodiments, there was positive immunostaining for the paneuronal marker β-tubulin III (TUJ1) (nerve fate). See, FIG. 13. In some embodiments, differentiation occurred over 7 days with monolayers present on the chip surface by day 8.

FIG. 13 shows that exemplary expression of neuronal lineage markers in the differentiating population of iNCs. NCM460/cHIMECs (upper row). NCM460/SMCs (middle row). Lower row NCM460. 40× magnification. From left to right columns: Hoechst stained nuclei colored blue. S100B glial cells colored green. TUJ1 neurons colored red. Composite images shows overlapping blue, green and red stained cells.

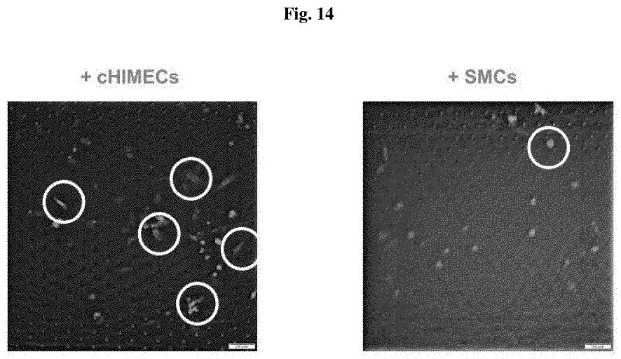

In some embodiments, functional characterization of differentiating iNC using Ca 2+ imaging on iNCs cultured with cHIMECs and iNCs cultured with smooth muscle cells, see FIG. 14.

FIG. 14 shows that exemplary functional characterization of differentiating iNC using Ca 2+ (colored green) imaging. Left panel shows cHIMECs. Right panel shows smooth muscle cells (SMCs).

In some embodiments, functional characterization for cells on chips include but are not limited to real-time readouts of cell morphology, cell motility, transport of fluorescent molecules across the tissue barrier, changes in function of reporter cell lines, Calcium ions in neuronal circuits, and optogenetic actuation.

In some embodiments, intestinal cells are commercially available intestinal cell lines. In some embodiments, intestinal epithelial cells are isolated from human biopsies and seeded as primary cells. In some embodiments, intestinal endothelial cells are isolated from human biopsies and seeded as primary cells. In some embodiments, intestinal smooth muscle cells are isolated from human biopsies and seeded as primary cells.

In some embodiments, iPS cells for producing iNCs are generated from human biopsy cells. In some embodiments, iPS cells for producing iNCs are generated from commercially available cells. In some embodiments, iPS cells for producing iNCs are commercially available cells.

An Innervated Gut-Chip with sensory neurons is contemplated to provide full morphological analysis for evidence that relevant cell types are organized in structures as seen in vivo and maintain morphological and functional characteristics upon 14 days of culture on the microfluidic chip. Each surface of a microfluidic chip will be coated with tissue specific extracellular matrix (e.g. Matrigel) to mimic the biologically active surfaces that cells normally sense in vivo. Cells will be plated, put on flow the next day, and will be maintained in the same conditions for two weeks to form confluent monolayers. Fluids will be continuously pumped through the channels to mimic shear forces, bring in nutrients, and wash away waste. In some embodiments, we will develop the model to include Human Intestinal Microvascular Endothelial cells (HIMECs) along the bottom channel. Colon epithelial cells (C2BBe1), resident immune cells provided by CD45+(Kerns et al., 2017) and iPSc derived sensory neurons will be seeded and grown on the upper channel of the Chips using protocols developed and validated at Emulate. Each surface is coated with tissue specific extracellular matrix (e.g. Collagen I-Lower channel, Matrigel-Upper channel) to mimic the biologically active surfaces that cells normally sense in vivo, as validated in our preliminary studies.

### The Following Read-Outs May be Applied to Chips:

Qualitative and quantitative characterization of human intestine endothelial cells will be done by immunostaining and imaging of cells growing in the Gut (intestine)-Chip with the following markers: VE-Cadherin, PECAM-1.

In some embodiments, evaluating tight junction formation on the epithelial monolayer is by immunostaining against ZO-1, E-cadherin, Occludin. Evaluating the barrier function at different time points (e.g. days: 3, 7, 14).

In some embodiments, vascular leakage will be measured as the vascular-to-epithelium diffusion of fluorescent fluid phase markers dextran (3 kDa) and Lucifer yellow (450 Da) and fluorescence will be detected via spectrophotometry. Following the addition of the fluorescents dyes on the apical channel, effluents from both apical and basal channels will be collected and apparent permeability (P app) will be calculated accordingly.

In some embodiments, characterizing the functional properties of the sensory neurons in the Intestine Chip is by calcium imaging (Fluo-4). In some embodiments, assess and characterize the functional properties of sensory neurons in our system by performing live calcium imaging (Fluo-4) throughout the experiment.

In some embodiments, apoptotic cells will undergo evaluation (Live-Dead staining, TUNEL cell death assay).

In some embodiments, RNAseq will be used to demonstrate expression of standard and specific markers for all cell types included on-chip and depict innervation-dependent gene expression. In some embodiments, RNAseq will be used for showing all cell types express standard and specific markers for their cell type.

In some embodiments

### Example A—Innervation Using iNeural Crest Cell Derived Neurons

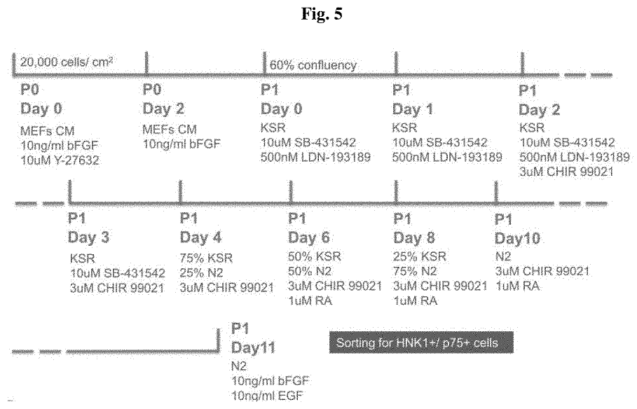

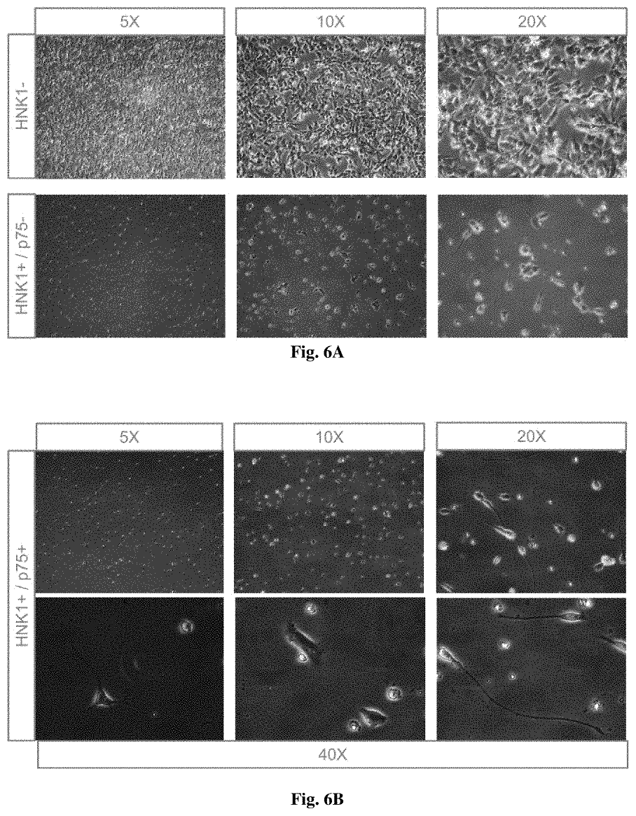

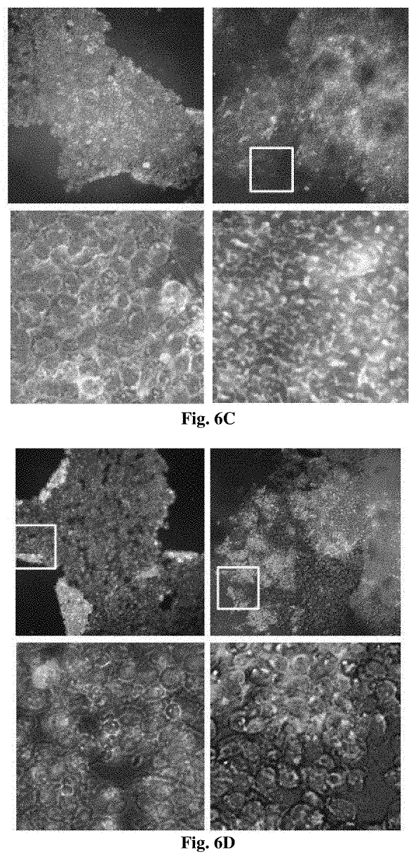

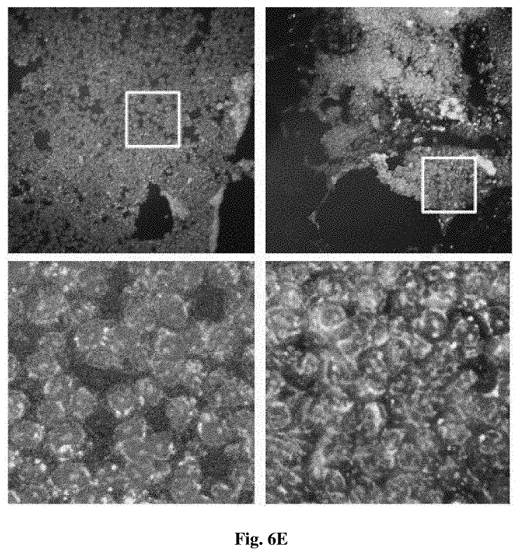

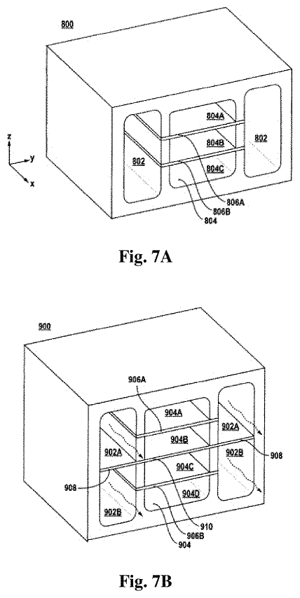

An exemplary protocol for Neural Crest differentiation includes (Chambers, et al., Meth in Mol Biol. 1307: 329-343, 2013), herein incorporated by reference in its entirety; mouse embryonic fibroblast-conditioned media (MEF-CM); and KSR Gibco™ KnockOut™ Serum Replacement (KnockOut™ SR). See, FIG. 5.

In one embodiment, glial cells and neuron cells may find use for innervation on chips. Glial cells (e.g. S100B+) and neuron cells (e.g. TUJ1+) were induced from HNK1+/p75+ sorted passage 1-Day 11 (P1d11) neural crest cell populations differentiated from PS cells (e.g. 20,000 cells/cm²). In one embodiment, beads were used for isolating (sorting out) HNK1+ plus p75+ cells. HNK1+ plus p75+ cells were then seeded onto a second membrane (lower) of a two-membrane chip. In one embodiment, Human Colonic Epithelial Cells (NCM460) were seeded on top of the upper (first membrane). In one embodiment, HNK1+ plus p75+ cells were seeded on top of Human Colonic Microvascular Epithelial Cells (cHIMECs). In one embodiment, cHIMECs are a source of NGF. In another embodiment, HNK1+ plus p75+ cells were seeded on top of Human Intestinal Smooth Muscle Cells (SMCs). In one embodiment, SMCs are a source of GDNF.

After 6 days of culture under flow with a Flow rate: 30 ul/hr, NCM460/cHIMECs and NCM460/SMCs showed S1000+ (glial cells) and TUJ1+ (neurons).

### Example B—Barrier Function—Electric Resistance

There are many ways to evaluate the integrity and physiology of an in vitro system that mimics a physiological barrier, e.g. an epithelial layer, an endothelial layer, a parenchymal cell barrier, etc. Two common methods are Transepithelial Electric Resistance (TEER) and dye particle diffusion measurements, e.g. Lucifer Yellow (LY) dye particle rejection, Luciferase Yellow, 450 Daltons (Da), Cascade blue particles, 3 kDa, etc., travel across cell monolayers. Because dye particle movement is based upon passive paracellular diffusion (through spaces between cells), dye particles have low permeability, i.e. movement, through intact cell layer barriers. Therefore, dye particles are considerably impeded in passing across cell monolayers with tight junctions. Permeability (Papp) for LY of <5 to 12 nm/s was reported to be indicative of a well-established monolayer having intact barrier function.

TEER measures the resistance to pass current across one or more cell layers on a membrane. Specifically, this electrical resistance is a direct measurement of the resistance of the cell monolayer to the transport of ions. The measurement may be affected by the pore size and density of the membrane, but it aims to ascertain cell and/or tissue properties. The TEER value is considered a good measure of the integrity of a cell monolayer.

Thus, in one embodiment, barrier function is monitored, including but not limited to innervated tissue on-chip.

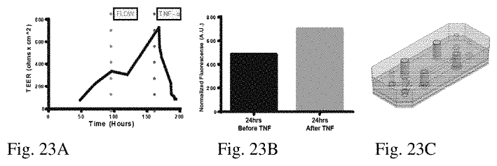

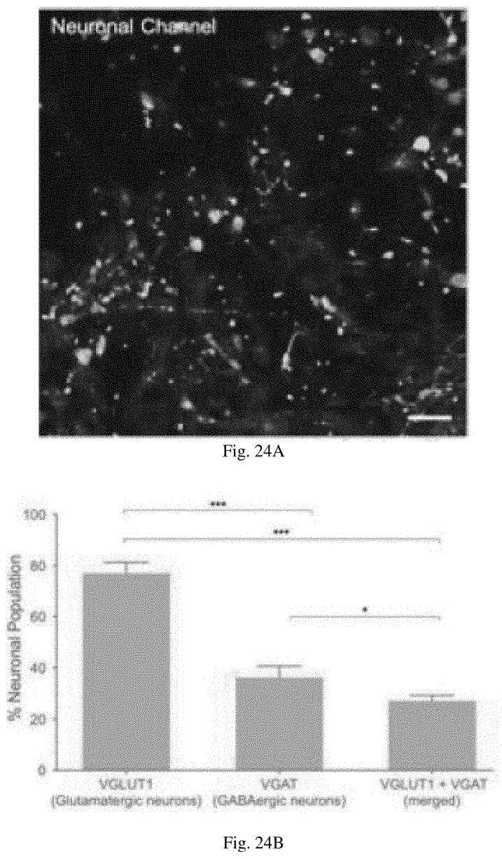

As one example, in one embodiment of a BBB-chip, such as described in Example B, barrier function was measured by flowing a fluorescent molecule on one side of the chip then measuring the amount that crosses the endothelial cell layer (FIG. 23B). The resolution of this measurement is limited by the time it takes for a sufficient sample of fluorescent molecules to flow through the endothelial cell layer of the chip.

FIG. 23A-C shows exemplary Real-time TEER measurements in and exemplary BBB-chip. FIG. 23A (left) shows a gradual increase in barrier function upon initiation of flow and then an abrupt drop after TNF-a challenge. The barrier function of the same BBB-chip measured by flowing a fluorescent molecule on one side of the chip and measuring how much of the molecules crosses the endothelial cells, FIG. 23B (middle). The resolution of this measurement is limited by the time it takes for a sufficient sample to flow through the chip. A model of an exemplary TEER-Chip design FIG. 23C (right) utilizing electrodes oriented in a manner that allows electrical resistance of the cells on the membrane to be measured in real-time.

### Example C—Stimulating Neurons for Electrical Measurements

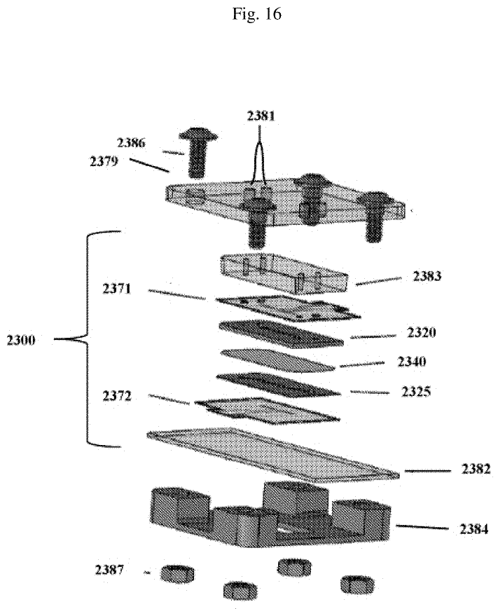

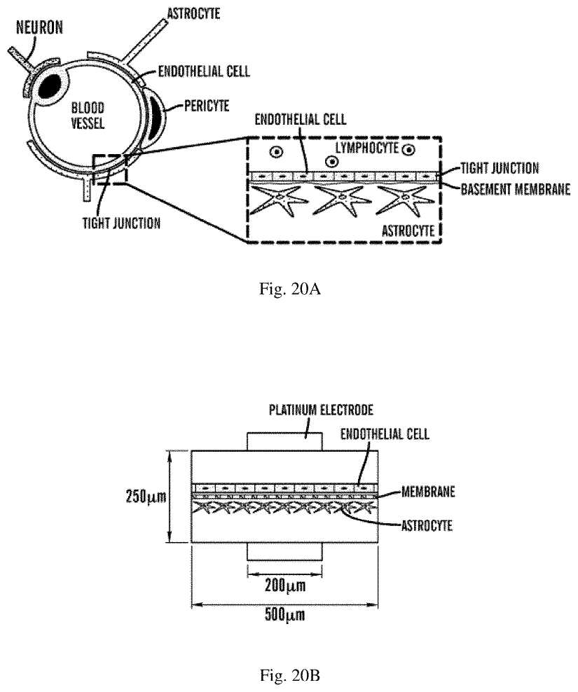

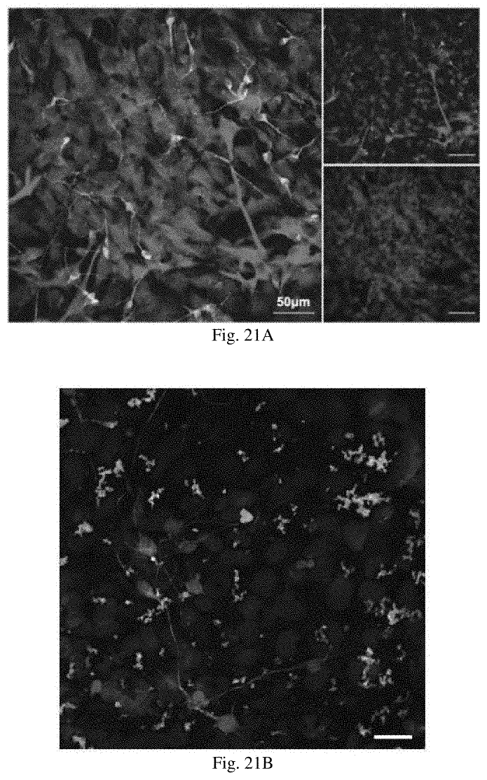

In one embodiment, a TEER-Chip comprising electrodes is used for testing compounds for inducing neuronal function by electrically stimulating innervated neurons using attached electrodes, embedded electrodes, etc. In one embodiment, electrodes are included in the neuronal compartment. In one embodiment, a TEER-Chip comprising electrodes is used for testing compounds for inducing neuronal function by electrically stimulating innervated neurons for inducing muscle twitch. Exemplary embodiments of attached electrodes are shown in FIGS. 16-20 and described herein.

In one embodiment, neurons are stimulated while directly stimulating the organ cells. In one embodiment, neurons are stimulated without directly stimulating the organ cells. Further, at least one of the advantages of using a fluidic device comprising a membrane, such that when directly stimulating cells on one side of a membrane, the membrane may prevent direct electrical stimulation of cells on the other side of the membrane. For example, electrodes may be attached to the neuronal compartment (or channel containing neuronal cells), such that the electrical stimulation predominantly does not get to the other side of the membrane or channel. As another example, applying a chemical/agent/drug/soluble factor to neuronal side in a way that predominantly does not make it to the other side due to the characteristics of the membrane.

### Example D—Inhibiting Neurons and/or Muscle Twitch

In one embodiment, a TEER-Chip comprising electrodes is used for testing compounds for inhibiting neuron function, i.e. Ca++ ion movement across membranes, electrical signaling, etc. In one embodiment, a TEER-Chip comprising electrodes is used for testing compounds for inhibiting muscle twitch.

Another nonlimiting example of a tool for real-time analysis of cellular function is integrated microscopy. This allows for real-time readouts of cell morphology, cell motility, transport of fluorescent molecules across the tissue barrier, changes in function of reporter cell lines, Calcium ions in neuronal circuits, etc. Exemplary embodiments of attached electrodes are shown in FIGS. 16-20 and described herein.

### Example E—Interactions of Epithelial Mucosa with Sensory Neurons and Microbiome

Previous studies showed a correlation between decreasing immune functions in humans with an increasing virulence of bacteria. Thus, in some embodiments, a human innervated Intestine-Chip (hiIC) is contemplated for use with the addition of immune cells and pathogenic bacteria. In some embodiments, the immune cell-innervated Intestine-Chip (hiIC) is used to test the immune response of this model with and without, or before and after, the addition of a pathogenic microorganism, such as a known pathogenic bacteria strain. In some embodiments, biological read outs include but are not limited to assessing the activation of the incorporated primary sensory neurons as an in vivo-relevant biological read out, disrupted barrier function, etc., for identifying bacteria-mediated disruption of the intestinal response (or defense) to pathogens. Such an understanding of the disruption of regulation of barrier function, etc., may be used in testing known and novel therapeutic targets for use in treatments for autoimmune associated microbial responses and infectious disorders of the intestine. Exemplary embodiments of attached electrodes are shown in FIGS. 16-20 and described herein.

### Example F—Innervated Brain-Chip

In one embodiment to provide an innervated human brain chip having biologically active surfaces where cells normally contact and sense in vivo, induced pluripotent stem cells (iPSC)-derived cortical neurons, including populations of Glutamatergic neurons and GABAergic neurons, human primary astrocytes, and human primary pericytes were seeded into the neuronal channel (upper chamber), and iPSC-derived human brain microvascular endothelial cells in the vascular channel (lower chamber).

In one embodiment of an innervated Brain-chip, human iPS-derived neurons are observed to be in direct contact with astrocytes and pericytes, after 10 days of co-culture. Moreover, co-cultures on-chip of human iPS-derived neurons further including microglia cells were observed to maintain cell specific morphology over time.

FIG. 21A shows an exemplary morphological characterization of the neuronal channel as one embodiment of an innervated Brain-chip. Scanning confocal microscope immunofluorescent images show immunohistochemistry staining of hiPSC-derived neuronal cultures using specific markers for discriminating neurons from astrocytes and pericytes, showing neurons (MPA2+, green) in direct contact with astrocytes (GFAP+, pink) and pericytes (NG2+, red), after 10 days of co-culture. Blue represents Hoechst-stained nuclei.

FIG. 21B shows an exemplary innervated Brain-chip further comprising microglia, demonstrating that microglia cells in neuron-microglial co-culture maintain their characteristics (ramified, referring to the cell body of the ramified form of microglia remains in place while its branches are constantly moving) on chip. Neurons (TUJ1-green), Microglia (Iba1-pink), Hoechst stain (nuclei-blue). Ionized calcium binding adaptor molecule 1 (Iba1) refers to a biomarker for a brain microglia calcium-binding protein.

FIG. 22A-D shows an exemplary morphological characterization of endothelial cells on a Brain chip by immunohistochemistry staining, shown as scanning confocal microscope immunofluorescent images in FIGS. 22A, 22C and 22D and flow cytometry analysis of endothelial cells FIG. 22D. Bar=50 μm.

FIG. 22A shows an exemplary immunocytochemical analysis of hiPSC-derived brain endothelial cells cultured on-chip for 7 days labeled with CD31 (white), F-actin (white), ZO-1 (green), GLUT-1 (red), Occludin (red), Claudin-5 (blue), and JAM-A (greenish-yellow). Bar=50 μm.

FIG. 22B shows an exemplary immunofluorescence micrographic view of the human brain microvascular endothelium cultured on-chip for 7 days demonstrating high levels of expression of ZO-1 across the entire endothelial cell monolayer. Bar=50 μm.

FIG. 22C shows an exemplary immunofluorescence micrographic side view of stacked images for the human Brain-Chip showing the lower chamber covered by a continuous monolayer of brain endothelial cells stained for Von Willebrand factor (vWF, Cyan), and a mixture of pericytes (NG2, red), astrocytes (GFAP, magenta), glutamatergic and GABAergic neurons (MAP2, green) on the surface of the porous membrane in the upper chamber of the same chip.

FIG. 22D shows an exemplary flow cytometry analysis demonstrating the expression of ZO-1, and GLUT-1.

### Example G—Innervated Brain-Chip Comprising Physiologically Relevant Ratios of Types of Cortical Neurons

In one embodiment to provide an innervated human brain chip having intact barrier function, induced pluripotent stem cells (iPSC)-derived cortical neurons, including populations of Glutamatergic neurons and GABAergic neurons, human primary astrocytes, and human primary pericytes were seeded into the neuronal channel (upper chamber), and iPSC-derived human brain microvascular endothelial cells in the vascular channel (lower chamber).

1. Seeding Human iPS-Derived Glutamatergic and GABAergic Neurons at High and Low Densities.

In some preferred embodiments, Glutamatergic and GABAergic neurons were seeded at different densities, e.g. high vs. low density, for comparing neuronal density and composition. Surprisingly, neuronal density and composition was discovered to influence the barrier function. For one example of evaluating neuronal density and composition, a comparison of Dextran leakage into the neuronal channel was compared between Brain-chips seeded with different densities of human iPSC-derived Glutamatergic neurons, GABAergic Neurons, or mixtures thereof.

Specifically, Glutamatergic Neurons alone were seeded at Low Density (0.4 mi/ml); GABAergic Neurons alone were seeded at Low Density (0.2 mi/ml); and a mixture of GABAergic Neurons and Glutamatergic Neurons (High Density) (0.8 mi/ml); and GABAergic Neurons and Glutamatergic Neurons (Low Density) (0.4 mi/ml), co-cultured for 3, 6 and 10 day readouts of % leakage into the neuronal channel. With the exception of GABAergic Neurons with Glutamatergic Neurons (high density) which showed increased leakage at 10 days, around 60%, and low density GABAergic Neurons alone around 30% leakage at 10 days; the remaining neuronal seedings and corresponding time periods showed comparable leakage, around 2-10% day 3, around 10-15% day 6, and around 15-20% day 10. Compared to Brain-chips without neurons, Brain-Chips with Low Density GABAergic+Glutamatergic Neurons showed higher rates of leakage over time, Days 6 to 10, however the difference was less than 20%.

2. Cortical Neuronal Composition Evaluated in Brain-Chips.

In one embodiment of a Brain-Chip, glutamatergic neurons were numerically dominant, whereas GABArgic cells constituted about 30-40% of total neurons in the Brain-Chip. Antibodies to vesicular glutamate transporter 1 (VGLUT1) and vesicular GABA transporter (VGAT), were used to identify each neuronal population, where overlapping labeling were used quantify overlapping glutamatergic+ and GABAergic+ labeling as presynaptic terminals. Additionally, Glutamate and GABA are observed to be co-expressed in a subset of cortical neurons, shown in FIG. 24B by approximately 80% VGLUT1+ neurons with approximately 30-40% VGAT+ cells, and approximately 30% double positive (merged) cells.

FIG. 24A-B shows an exemplary immunofluorescence micrographic view and quantitation of a Glutamatergic/GABAergic ratio observed in the Brain-Chip that has an in vitro relevance with the in vivo human cerebral cortex.

FIG. 24A shows exemplary glutamatergic neurons identified using vesicular glutamate transporter 1 (VGLUT1) antibodies (red) and GABArgic neurons identified using vesicular GABA transporter (VGAT) antibodies (green). Overlaping neurons, yellow, identify glutamatergic and GABAergic presynaptic terminals. Bar=50 μm.

FIG. 24B shows exemplary graphical quantitation as a percentage of the neuronal population on-chip.

3. Permeability of the Brain-Chip in Relation to Types of Cortical Neurons.

One embodiment of a Brain-Chip model showed physiologically relevant vascular-to-brain diffusion, i.e. astrocytes and pericytes seeded with iPS derived glutamatergic neurons and GABArgic neurons, in co-cultures with endothelial cells. Physiological ratios of glutamatergic and GABAergic compositions strengthen the barrier integrity, whereas individual glutamatergic or GABAergic neurons in combination with astrocytes, pericytes and endothelial cells do not.

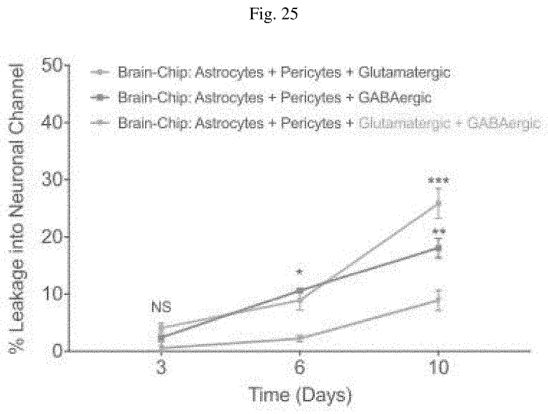

FIG. 25 shows an exemplary assessment of the permeability in several embodiments of a Brain-Chip, e.g. astrocytes and pericytes seeded with iPS derived 1) glutamatergic neurons; 2) GABArgic neurons or 3) with both glutamatergic neurons and GABArgic neurons. A known concentration of a fluorescent protein, Dextran, was added in the vascular channel media; then after a defined period of time, the fluorescent Dextran was quantified in the neuronal channel via effluent.

4. Embodiments of an Inflamed Brain-Chip.

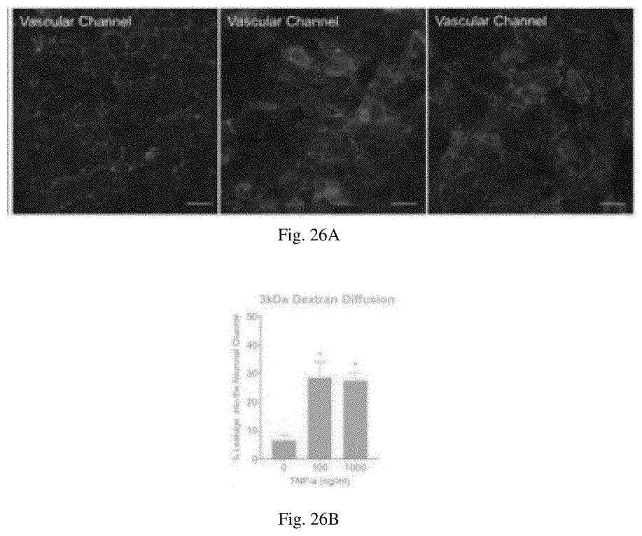

FIG. 26A-B shows an exemplary creation of an inflammatory environment on a Brain-chip. The vascular channel was directly stimulated the vascular channel at two concentrations of TNF-α. Exposure to TNF-α for 16 hours induces changes in endothelial cell morphology, and significantly increases the paracellular permeability.

FIG. 26A shows exemplary morphological disruption of endothelial cell expression of ZO-1, control—left panel, TNF-α (100 ng/ml)—middle panel and TNF-α 1 ug/ml—right panel, viewed as immunofluorescent micrographs. Bar=50 μm.

FIG. 26B shows exemplary quantitative correlation of a loss in barrier function, indicated by the disrupted ZO-1 expression. The percent leakage of Dextran into the apical channel is significantly higher than the control for both levels of TNF-α treatment.

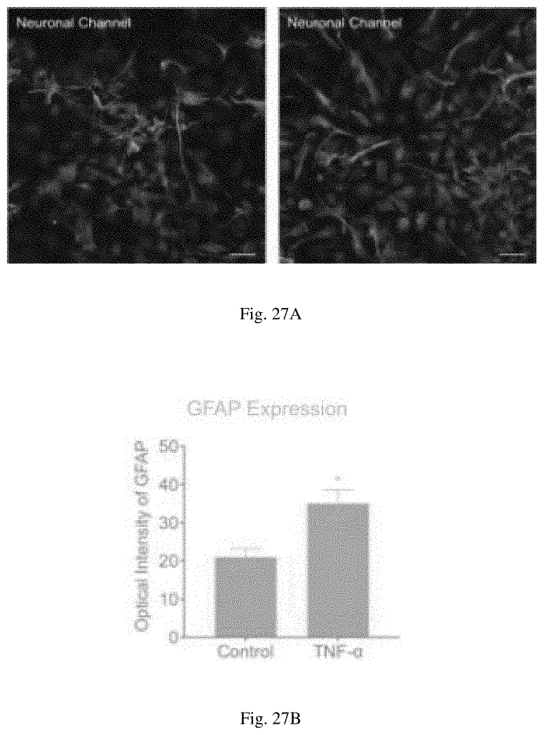

FIG. 27A-B shows exemplary TNF-α treatment of one embodiment of a Brain-Chip significantly increases the GFAP expression (i.e. by astrocytes) up to 24 hours after stimulation. TNF-α (100 ng/ml) or vehicle was perfused into the neuronal channel.

FIG. 27A shows exemplary immunofluorescent micrographs of the neuronal channel demonstrating an increase in GFAP expression (pink); MAP2 (green); and Hoechst nuclear stain (blue) for vehicle, left panel (control) vs. TNF-α treatment, right panel.

FIG. 27A shows an exemplary graphical quantification of optical intensity of GFAP staining showing TNF-α (100 ng/ml) significantly increases the GFAP expression up to 24 hours after stimulation.

Thus, in some preferred embodiments, numbers of Glutamatergic and GABAergic neurons are within a range of physiologically relevant ratios, e.g. relevant to ratios measured in vivo within the human cerebral cortex, Thus, in one embodiment, an innervated Brain chip mimics the blood brain barrier of a human cerebral cortex. In some embodiments, Brain chips do not have embedded electrodes. In some embodiments, Brain chips do have embedded electrodes.

### Example H—Chemical Capsaicin Stimulation of Neurons On-Chip

In one embodiment, a chemical may be used to stimulate innervated neurons on-chip. In one embodiment, a microfluidic chip is referred to as a “Pain-chip” or “Nociceptors-on-Chip” or “Nociceptors-Chip.”

Merely as an example, Capsaicin, e.g. (trans-8-methyl-N-vanillyl-6-nonenamide) may be used to provide a model for inducing a pain stimulus in a Nociceptors-Chip. Thus, a capsaicin (pain) induced model using human iPS-derived sensory neurons (Nociceptors) that “sense” pain with clinically relevant readouts, e.g. endpoints, (such as elevated calcium levels, Substance P secretion, etc., was developed as described herein.

Thus, in one embodiment, human iPS-derived sensory neurons (Nociceptors) were differentiated on-chip in the apical channel as described herein. In particular, the apical channel was coated with Laminin 10 μg/ml, then incubated at 4° C. overnight. Unattached laminin was washed away with PBS, then culture medium was flowed in to cover the coated membrane to replace the PBS. Seeded iPS cells were not embedded in a gel.

In one embodiment, endothelial cells were seeded into the basal channel, e.g. Human Dermal Microvascular Endothelial Cells (HMVEC-D), i.e. Primary Dermal Microvascular Endothelial Cells; Normal, Human, Neonatal (HDMVECn) (e.g. ATCC® PCS-110-010™). However, it is not meant to limit the source of HMVEC, such that Primary Dermal Microvascular Endothelial Cells may be obtained from neonatal biopsies, e.g. foreskins. Prior to seeding, basal channels were coated with a thin rat collagen coating. Rat collagen was diluted in distillated water to obtain a final protein concentration at 150 μg/mL−1. The basal channel was coated by incubating the channel in collagen solution at 4° C. overnight. Unattached rat collagen was washed away with PBS, then culture medium was flowed in to cover the coated membrane to replace the PBS.

After seeding iPS cells and endothelial cells, microfluidic chips were incubated under conditions for generating differentiated human iPS-derived sensory neurons (Nociceptors), as described herein.

Such differentiated co-cultures of sensory neurons on-chip maintained their characteristics after 7 days in culture under flow, apical channel flow rate at 30 μl/h for 7 days and simultaneously, endothelial cells in the basal channel were incubated under flow at 60 μl/h for 7 days. See, FIG. 28A-C.

Irritation (as pain simulation) to sensory neurons on-chip was induced by capsaicin (1 μM in DMEM/F12 with 10% FBS, supplemented with BDNF, GDNF, NGF, NT-3 (25 ng/ml each) and Ascorbic Acid (200 μM)) in order to recapitulate the sensation of burning (heat) felt by humans exposed to capsaicin.

Nociceptors responded to capsaicin, as shown by live imaging of calcium using Fluo-4 AM, see FIGS. 28C and 28D.

Fluo-4 AM refers to a calcium sensitive fluorescent dye, loaded onto the sensory neurons. Intracellular calcium fluctuations were monitored over time using high-speed imaging. Acquisition protocols, for the example shown herein, consisted of 5-minute time-lapse sequences of Fluo-4 fluorescence. Alterations in fluorescence as a function of time were measured at a single wavelength (Fluo-4). Analysis and processing, as well as playback of the image sequences for visual inspection, was made using ImageJ/FIJI software. To visualize the spatial and temporal changes in, the raw sequences were processed to highlight changes in fluorescence intensity between frames. Regions of interest over the field of view were selected, and the mean pixel intensity at each frame was measured. The data was first plotted as fluorescence intensity versus and subsequently converted to a relative scale (ΔF/F baseline)

FIG. 28A-D shows exemplary sensory neurons, i.e. Nociceptors, in an apical channel on-chip, maintaining their neuronal characteristics on-chip after 7 days in culture.

FIG. 28A shows a fluorescent image of the entire neuronal channel showing Tuj-1+ neurons (green) upper panel; TRPV1 neurons (red) middle panel; and merged images showing yellow co-staining of Nociceptors, lower panel.

FIG. 28B shows exemplary Nociceptors as yellow co-stained Tuj-1+/TRPV1+ neurons, left panel, Tuj-1+(green) upper right; TRPV1+ (red) lower right. Scale bars=50 μm.

FIG. 28C shows exemplary Nociceptors stained with Fluo4-AM after irritation by Capsaicin (1 μM), see circled activated nodes.

FIG. 28D shows an exemplary graph of Nociceptors responding to capsaicin, as shown by live imaging of calcium, change in fluorescence (ΔF) compared to fluorescence before stimulation (F).

Chemical stimulation of Nociceptors with Capsaicin (e.g. 1 μM), perfused for 3 hours, was followed by effluent collected from the apical channel (neuronal). This Capsaicin treatment elicited the release of substance P, a pain neurotransmitter. Thus demonstrating the ability of an innervated chip to “Sense” with clinically-relevant endpoints, e.g. calcium flux, above, and release of substance P, see, FIGS. 23A-B.

FIG. 29A-B shows an exemplary read-out of substance P produced by chronic stimulation of Nociceptors (sensory neurons) though perfusion of the neuronal channel with capsaicin.

FIG. 29A shows an exemplary schematic of a closed channel chip comprising nociceptors in the apical channel perfused with capsaicin (1 uM) through the inlet, under flow, where the effluent was collected from the outlet of the apical channel after 3 hours (t=3 h).

FIG. 29B shows an exemplary graph of Substance P (pg/ml) measured in the apical channel effluent, as described in FIG. 23A showing a significant increase in Substance P released after capsaicin.

This model was further developed, as described herein, successfully simulating the process of sensitization that leads to pathological pain by the addition of inflammatory mediators to endothelial media perfused through the vascular channel. As one example, 5 μM each of Prostaglandin E2, bradykinin, serotonin and histamine in Vascular Cell Basal Medium (ATCC® PCS-100-030™) with Microvascular Endothelial Cell Growth Kit-VEGF (ATCC® PCS-110-041™) was perfused through the vascular channel. In other words, this model is intended to simulate peripheral sensitization of Nociceptors. Capsaicin (200 nM) and inflammatory mediators (each 5 μM) were perfused for 3 hours, and the effluents were collected from the apical channel (neuronal). See, FIG. 30A for an exemplary schematic diagram.

As shown below, sensitization of sensory neurons with capsaicin (e.g. 200 nM) resulted in significant increased release of substance P along with a significant increase in vascular permeability, see FIG. 30B and FIG. 31C.

FIG. 30A-B shows an exemplary read-out of substance P produced by chronic stimulation of Nociceptors (sensory neurons) though perfusion of the neuronal channel with capsaicin in the presence of inflammatory mediators simultaneously perfused through the basal channel.

FIG. 30A shows an exemplary schematic of a closed channel chip comprising Nociceptors in the apical channel perfused with capsaicin (200 nM in media) through the inlet, under flow. At the same time, inflammatory mediators, i.e. Prostaglandin E2, bradykinin, serotonin and histamine (each 5 μM in media), were added to the inlet of the basal channel. The effluent was collected from the outlet of the apical channel after 3 hours (t=3 h).

FIG. 30B shows an exemplary graph of Substance P (pg/ml) measured in the apical channel effluent, as described in FIG. 29A showing a significant increase in Substance P released after capsaicin treatment in the presence of inflammatory mediators.

Thus, Nociceptors become sensitized by inflammatory mediators and disperse considerably more substance P in the presence of low levels of Capsaicin.

Therefore, another readout of “pain” stimulated sensory neurons is measuring the effect of Nociceptors (e.g. results of producing substance P, see above) on vascular permeability. Thus, in one embodiment, vascular permeability is indicated by VE-cadherin staining of endothelial cells in the basal (vascular) channel. See, FIG. 31A.

FIG. 31A-B shows an exemplary read-out of changes in vascular permeability produced by chronic stimulation of Nociceptors (sensory neurons) though perfusion of the neuronal channel with capsaicin in the presence of inflammatory mediators simultaneously perfused through the basal channel.

FIG. 31A shows exemplary florescent microscope images of endothelial cells in the basal channel. Left, Hoechst staining of nuclei (blue), middle, e-cadherin staining (pink) and right, merged florescent images showing individual cells (blue nuclei) surrounded by e-cadherin.

Vascular endothelial (VE)-cadherin refers to a major adhesion molecule in endothelial cells. Similar to other classical cadherins, the cytoplasmic tail of VE-cadherin associates with various intracellular proteins including β-catenin and p120 catenin. Furthermore, the connection between adherens junctions and actin filaments mediated by VE-cadherin is believed to be crucial for the regulation of blood vascular endothelial functions, including cellular reactions to various endothelial permeability factors and angiogenic growth factors. For further information see, Dejana, et al., “The control of vascular integrity by endothelial cell junctions: Molecular basis and pathological implications.” Dev Cell 16:209-221, 2009; and Vestweber, “VE-cadherin: The major endothelial adhesion molecule controlling cellular junctions and blood vessel formation.” Arterioscler Thromb Vasc Biol; 28:223-232, 2008.

Another readout is obtained by using Dextran (3 kDa) permeability, where Dextran is added to the basal channel then after 24 hours, effluent from the apical channel was collected for measuring the amount of Dextran that diffused through the endothelial barrier into the neuronal channel. See, FIG. 31B.

FIG. 31B shows an exemplary schematic of a vertical chip cross-section of a closed channel chip comprising Nociceptors in the apical channel separated from HMVEC-D by a membrane (yellow) where Dextran (3 kDa—blue dots) added to the basal channel leaked into the apical channel.

Activation of Nociceptors, in the simultaneous presence of Capsaicin and inflammatory mediators triggered the local release of neuropeptides, such as substance P, as described above. Further, the simultaneous presence of Capsaicin and inflammatory mediator induced a significant increase in vascular permeability with stimulation of vasodilation (neurogenic inflammation) on-chip. See, FIG. 31C.

FIG. 31C shows an exemplary graphical comparison of leakage (percent) of 3 kDa Dextrin into the apical channel between controls, no treatment (left), inflammatory mediators (middle), demonstrating a significant amount of leakage after perfusion with capsaicin (200 nM in media) in the apical channel simultaneously with perfusion of inflammatory mediators, i.e. Prostaglandin E2, Bradykinin, serotonin and histamine (each 5 μM in media), added to the inlet of the basal channel, right, after 3 hours (t=3 h).

### DETAILED DESCRIPTION OF INVENTION

It is not meant to limit the types of fluidic devices intended for innervation. Exemplary fluidic devices are described herein that may be used for specific aspects of the invention. The present invention contemplates a variety of configurations and methods of using these fluidic devices.

### I. Microfluidic Chips, Devices and Systems.

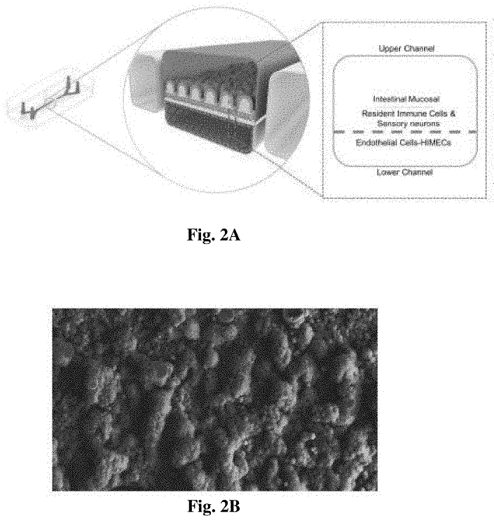

Microfluidic chips, fluidic devices, and systems contemplated for use include but are not limited to chips described in Bhatia and Ingber, “Microfluidic organs-on-chips.” Nature Biotechnology, 32(8):760-722, 2014; and related patent applications; and further include a wide range of chips of which some are briefly described in Zhang and Radisic,” Organ-on-a-chip devices advance to market.” Lab On A Chip, (2017), for some examples, see FIG. 2 in Zhang and Radisic, herein incorporated by reference in their entireties. The following section is merely for providing nonlimiting examples of embodiments that may find use as microfluidic devices.

In some embodiments, fluidic devices are closed-top systems. In some embodiments, fluidic devices are open-top systems. In some embodiments, fluidic devices comprise some components of both closed-top systems and open-top systems. In particular, embodiments of fluidic devices comprise structural anchors, in part for providing intact gel layers, stretchable membranes, etc. In some embodiments, fluidic devices comprise electrodes for taking real-time physiological measurements, such as neuronal electrical signals; for taking real-time TEER measurements for barrier function, etc. In some embodiments, fluidic devices comprise stretchable membranes with electrodes for taking both real-time TEER measurements and real-time physiological measurements.

In one embodiment, a TEER-Chip utilizing electrodes oriented in a manner so that only the electrical resistance of the cells on the membrane to be measured in real-time is described in Odijk, 2015. In one embodiment, a TEER-Chip utilizing electrodes oriented in a manner so that only the electrical resistance of the cells on the membrane to be measured in real-time is described in WO 2017/096297, published 8 Jun. 2017.

### II. Closed Top Chips.

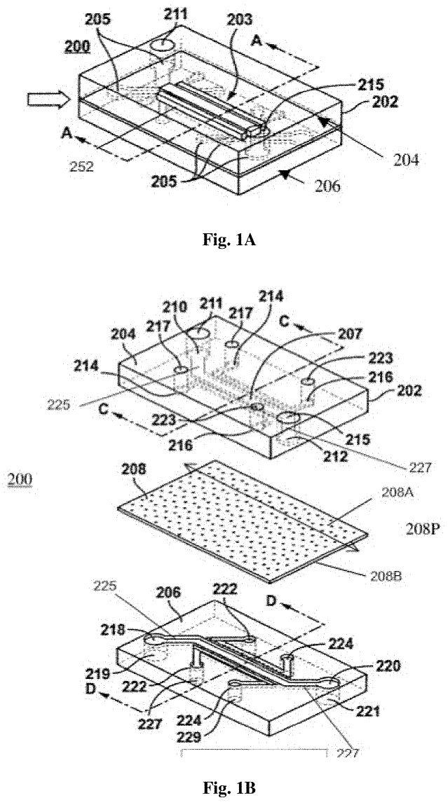

In some embodiments, the present disclosure relates to closed-top fluidic device, e.g. exemplary schematics in FIGS. 1A-B. The present disclosure relates to gut-on-chips, such as fluidic devices comprising one or more cells types for the simulation one or more of the function of gastrointestinal tract components. Accordingly, the present disclosure additionally describes closed-top intestine-on-chips, see, e.g. schematic in FIG. 1A-B.

The present disclosure also relates to BBB (blood brain barrier)-on-chips, which may also use a fluidic device such as depicted schematically in FIGS. 1A-B.

The present disclosure additionally relates to fluidic devices comprising cells described herein as part of closed-top devices.

FIGS. 1A-B illustrates a perspective view of the devices in accordance with some embodiments described herein. For example, as shown in FIGS. 1A-1B, the device 200 can include a body 202 comprising a first structure 204 and a second structure 206 in accordance with an embodiment. The body 202 can be made of an elastomeric material, although the body can be alternatively made of a non-elastomeric material, or a combination of elastomeric and non-elastomeric materials. It should be noted that the microchannel design 203 is only exemplary and not limited to the configuration shown in FIGS. 1A-1B. While operating chambers 252 (e.g., as a pneumatics means to actuate the membrane 208, see the International Appl. No. PCT/US2009/050830 for further details of the operating chambers, the content of which is incorporated herein by reference in its entirety) are shown in FIGS. 1A-1B, they are not required in all of the embodiments described herein. In some embodiments, the devices do not comprise operating chambers on either side of the first chamber and the second chamber. In other embodiments, the devices described herein can be configured to provide other means to actuate the membrane, e.g., as described in the International Pat. Appl. No. PCT/US2014/071570, the content of which is incorporated herein by reference in its entirety.

In some embodiments, various organ chip devices described in the International Patent Application Nos. PCT/US2009/050830; PCT/US2012/026934; PCT/US2012/068725; PCT/US2012/068766; PCT/US2014/071611; and PCT/US2014/071570, the contents of each of which are incorporated herein by reference in their entireties, can be modified to form the devices described herein. For example, the organ chip devices described in those patent applications can be modified in accordance with the devices described herein.

The first structure 204 and/or second structure 206 can be fabricated from a rigid material, an elastomeric material, or a combination thereof. As used herein, the term “rigid” refers to a material that is stiff and does not bend easily, or maintains very close to its original form after pressure has been applied to it. The term “elastomeric” as used herein refers to a material or a composite material that is not rigid as defined herein. An elastomeric material is generally moldable and curable, and has an elastic property that enables the material to at least partially deform (e.g., stretching, expanding, contracting, retracting, compressing, twisting, and/or bending) when subjected to a mechanical force or pressure and partially or completely resume its original form or position in the absence of the mechanical force or pressure. In some embodiments, the term “elastomeric” can also refer to a material that is flexible/stretchable but does not resume its original form or position after pressure has been applied to it and removed thereafter. The terms “elastomeric” and “flexible” are interchangeably used herein.

In some embodiments, the material used to make the first structure and/or second structure or at least the portion of the first structure 204 and/or second structure 206 that is in contact with a gaseous and/or liquid fluid can comprise a biocompatible polymer or polymer blend, including but not limited to, polydimethylsiloxane (PDMS), polyurethane, polyimide, styrene-ethylene-butylene-styrene (SEBS), polypropylene, polycarbonate, cyclic polyolefin polymer/copolymer (COP/COC), or any combinations thereof. As used herein, the term “biocompatible” refers to any material that does not deteriorate appreciably and does not induce a significant immune response or deleterious tissue reaction, e.g., toxic reaction or significant irritation, over time when implanted into or placed adjacent to the biological tissue of a subject, or induce blood clotting or coagulation when it comes in contact with blood.

Additionally or alternatively, at least a portion of the first structure 204 and/or second structure 206 can be made of non-flexible or rigid materials like glass, silicon, hard plastic, metal, or any combinations thereof.

The membrane 208 can be made of the same material as the first structure 204 and/or second structure 206 or a material that is different from the first structure 204 and/or second structure 206 of the devices described herein. In some embodiments, the membrane 208 can be made of a rigid material. In some embodiments, the membrane is a thermoplastic rigid material. Examples of rigid materials that can be used for fabrication of the membrane include, but are not limited to, polyester, polycarbonate or a combination thereof. In some embodiments, the membrane 208 can comprise a flexible material, e.g., but not limited to PDMS. Additional information about the membrane is further described below.

In some embodiments, the first structure and/or second structure of the device and/or the membrane can comprise or is composed of an extracellular matrix polymer, gel, and/or scaffold. Any extracellular matrix can be used herein, including, but not limited to, silk, chitosan, elastin, collagen, proteoglycans, hyaluronic acid, collagen, fibrin, and any combinations thereof

The device in FIG. 1A can comprise a plurality of access ports 205. In addition, the branched configuration 203 can comprise a tissue-tissue interface simulation region (membrane 208 in FIG. 1B) where cell behavior and/or passage of gases, chemicals, molecules, particulates and cells are monitored.

FIG. 1B illustrates an exploded view of the device in accordance with an embodiment. In one embodiment, the body 202 of the device 200 comprises a first outer body portion (first structure) 204, a second outer body portion (second structure) 206 and an intermediary membrane 208 configured to be mounted between the first and second outer body portions 204, 206 when the portions 204, 206 are mounted to one another to form the overall body.

The first outer body portion or first structure 204 can have a thickness of any dimension, depending, in part, on the height of the first chamber 204. In some embodiments, the thickness of the first outer body portion or first structure 204 can be about 1 mm to about 100 mm, or about 2 mm to about 75 mm, or about 3 mm to about 50 mm, or about 3 mm to about 25 mm. In some embodiments, the first outer body portion or first structure 204 can have a thickness that is more than the height of the first chamber by no more than 5 mm, no more than 4 mm, no more than 3 mm, no more than 2 mm, no more than 1 mm, no more than 500 microns, no more than 400 microns, no more than 300 microns, no more than 200 microns, no more than 100 microns, no more than 70 microns or less. In some embodiments, it is desirable to keep the first outer body portion or first structure 204 as thin as possible such that cells on the membrane can be visualized or detected by microscopic, spectroscopic, and/or electrical sensing methods.

The second outer body portion or second structure 206 can have a thickness of any dimension, depending, in part, on the height of the second chamber 206. In some embodiments, the thickness of the second outer body portion or second structure 206 can be about 50 μm to about 10 mm, or about 75 μm to about 8 mm, or about 100 μm to about 5 mm, or about 200 μm to about 2.5 mm. In one embodiment, the thickness of the second outer body portion or second structure 206 can be about 1 mm to about 1.5 mm. In one embodiment, the thickness of the second outer body portion or second structure 206 can be about 0.2 mm to about 0.5 mm. In some embodiments, the second outer first structure and/or second structure portion 206 can have a thickness that is more than the height of the second chamber by no more than 5 mm, no more than 4 mm, no more than 3 mm, no more than 2 mm, no more than 1 mm, no more than 500 microns, no more than 400 microns, no more than 300 microns, no more than 200 microns, no more than 100 microns, no more than 70 microns or less. In some embodiments, it is desirable to keep the second outer body portion or second structure 206 as thin as possible such that cells on the membrane can be visualized or detected by microscopic, spectroscopic, and/or electrical sensing methods.

In some embodiments, the first chamber and the second chamber can each independently comprise a channel. The channel(s) can be substantially linear or they can be non-linear. In some embodiments, the channels are not limited to straight or linear channels and can comprise curved, angled, or otherwise non-linear channels. It is to be further understood that a first portion of a channel can be straight, and a second portion of the same channel can be curved, angled, or otherwise non-linear. Without wishing to be bound by a theory, a non-linear channel can increase the ratio of culture area to device area, thereby providing a larger surface area for cells to grow. This can also allow for a higher amount or density of cells in the channel.

FIG. 1B illustrates an exploded view of the device in accordance with an embodiment. As shown in FIG. 1B, the first outer body portion 204 includes one or more inlet fluid ports 210 preferably in communication with one or more corresponding inlet apertures 211 located on an outer surface of the body 202. The device 100 is preferably connected to the fluid source 104 via the inlet aperture 211 in which fluid travels from the fluid source 104 into the device 100 through the inlet fluid port 210.

Additionally, the first outer body portion or first structure 204 can include one or more outlet fluid ports 212 in communication with one or more corresponding outlet apertures 215 on the outer surface of the first structure 204. In some embodiments, a fluid passing through the device 200 can exit the device to a fluid collector or other appropriate component via the corresponding outlet aperture 215. It should be noted that the device 200 can be set up such that the fluid port 210 is an outlet and fluid port 212 is an inlet.

In some embodiments, as shown in FIG. 29B, the device 200 can comprise an inlet channel 225 connecting an inlet fluid port 210 to the first chamber 204. The inlet channels and inlet ports can be used to introduce cells, agents (e.g., but not limited to, stimulants, drug candidate, particulates), airflow, and/or cell culture media into the first chamber 204.

The device 200 can also comprise an outlet channel 227 connecting an outlet fluid port 212 to the first chamber 204. The outlet channels and outlet ports can also be used to introduce cells, agents (e.g., but not limited to, stimulants, drug candidate, particulates), airflow, and/or cell culture media into the first chamber 204.

Although the inlet and outlet apertures 211, 215 are shown on the top surface of the first structure 204 and are located perpendicular to the inlet and outlet channels 225, 227, one or more of the apertures 211, 215 can be located on one or more lateral surfaces of the first structure and/or second structure such that at least one of the inlet and outlet apertures 211, 215 can be in-plane with the inlet and/or outlet channels 225, 227, respectively, and/or be oriented at an angle from the plane of the inlet and/or outlet channels 225, 227.

In another embodiment, the fluid passing between the inlet and outlet fluid ports can be shared between the first chamber 204 and second chamber 206. In either embodiment, characteristics of the fluid flow, such as flow rate, fluid type and/or composition, and the like, passing through the first chamber 204 can be controllable independently of fluid flow characteristics through the second chamber 206 and vice versa.

In some embodiments, while not necessary, the first structure 204 can include one or more pressure inlet ports 214 and one or more pressure outlet ports 216 in which the inlet ports 214 are in communication with corresponding apertures 217 located on the outer surface of the device 200. Although the inlet and outlet apertures are shown on the top surface of the first structure 204, one or more of the apertures can alternatively be located on one or more lateral sides of the first structure and/or second structure. In operation, one or more pressure tubes (not shown) connected to an external force source (e.g., pressure source) can provide positive or negative pressure to the device via the apertures 217. Additionally, pressure tubes (not shown) can be connected to the device 200 to remove the pressurized fluid from the outlet port 216 via the apertures 223. It should be noted that the device 200 can be set up such that the pressure port 214 is an outlet and pressure port 216 is an inlet. It should be noted that although the pressure apertures 217, 223 are shown on the top surface of the first structure 204, one or more of the pressure apertures 217, 223 can be located on one or more side surfaces of the first structure 204.

Referring to FIG. 1B, in some embodiments, the second structure 206 can include one or more inlet fluid ports 218 and one or more outlet fluid ports 220. As shown in FIG. 29B, the inlet fluid port 218 is in communication with aperture 219 and outlet fluid port 220 is in communication with aperture 221, whereby the apertures 219 and 221 are located on the outer surface of the second structure 206. Although the inlet and outlet apertures are shown on the surface of the second structure, one or more of the apertures can be alternatively located on one or more lateral sides of the second structure.

As with the first outer body portion or first structure 204 described above, one or more fluid tubes connected to a fluid source can be coupled to the aperture 219 to provide fluid to the device 200 via port 218. Additionally, fluid can exit the device 200 via the outlet port 220 and outlet aperture 221 to a fluid reservoir/collector or other component. It should be noted that the device 200 can be set up such that the fluid port 218 is an outlet and fluid port 220 is an inlet.

In some embodiments, the second outer body portion and/or second structure 206 can include one or more pressure inlet ports 222 and one or more pressure outlet ports 224. In some embodiments, the pressure inlet ports 222 can be in communication with apertures 227 and pressure outlet ports 224 are in communication with apertures 229, whereby apertures 227 and 229 are located on the outer surface of the second structure 206. Although the inlet and outlet apertures are shown on the bottom surface of the second structure 206, one or more of the apertures can be alternatively located on one or more lateral sides of the second structure. Pressure tubes connected to an external force source (e.g., pressure source) can be engaged with ports 222 and 224 via corresponding apertures 227 and 229. It should be noted that the device 200 can be set up such that the pressure port 222 is an outlet and fluid port 224 is an inlet.

In some embodiments where the operating channels (e.g., 252 shown in FIG. 1A) are not mandatory, the first structure 204 does not require any pressure inlet port 214, pressure outlet port 216. Similarly, the second structure 206 does not require any pressure inlet port 222 or pressure outlet port 224.

In an embodiment, the membrane 208 is mounted between the first structure 204 and the second structure 206, whereby the membrane 208 is located within the first structure 204 and/or second structure 206 of the device 200. In an embodiment, the membrane 208 is a made of a material having a plurality of pores or apertures therethrough, whereby molecules, cells, fluid or any media is capable of passing through the membrane 208 via one or more pores in the membrane 208. As discussed in more detail below, the membrane 208 in one embodiment can be made of a material which allows the membrane 208 to undergo stress and/or strain in response to an external force (e.g., cyclic stretching or pressure). In one embodiment, the membrane 208 can be made of a material, which allows the membrane 208 to undergo stress and/or strain in response to pressure differentials present between the first chamber 204, the second chamber 206 and the operating channels 252. Alternatively, the membrane 208 is relatively inelastic or rigid in which the membrane 208 undergoes minimal or no movement.

In some embodiments where the device simulates a function of a tissue, such as a lymph node, the membrane can be rigid.

The first chamber 204 and/or the second chamber 206 can have a length suited to the need of an application (e.g., a physiological system to be modeled), desirable size of the device, and/or desirable size of the view of field. In some embodiments, the first chamber 204 and/or the second chamber 206 can have a length of about 0.5 cm to about 10 cm. In one embodiment, the first chamber 204 and/or the second chamber 206 can have a length of about 1 cm to about 3 cm. In one embodiment, the first chamber 204 and/or the second chamber 206 can have a length of about 2 cm.

The width of the first chamber and/or the second chamber can vary with desired cell growth surface area. The first chamber 204 and the second chamber 206 can each have a range of width dimension between 100 microns and 50 mm, or between 200 microns and 10 mm, or between 200 microns and 1500 microns, or between 400 microns and 1 mm, or between 50 microns and 2 mm, or between 100 microns and 5 mm. In some embodiments, the first chamber 204 and the second chamber 206 can each have a width of about 500 microns to about 2 mm. In some embodiments, the first chamber 204 and the second chamber 206 can each have a width of about 1 mm.

In some embodiments, the widths of the first chamber and the second chamber can be configured to be different, with the centers of the chambers aligned or not aligned. In some embodiments, the channel heights, widths, and/or cross sections can vary along the length of the devices described herein.

The heights of the first chamber and the second chamber can vary to suit the needs of desired applications (e.g., to provide a low shear stress, and/or to accommodate cell size). The first chamber and the second chamber of the devices described herein can have the same heights or different heights. In some embodiments, the height of the second chamber 206 can be substantially the same as the height of the first chamber 204.

In some embodiments, the height of at least a length portion of the first chamber 204 (e.g., the length portion where the cells are designated to grow) can be substantially greater than the height of the second chamber 206 within the same length portion. For example, the height ratio of the first chamber to the second chamber can be greater than 1:1, including, for example, greater than 1.1:1, 1.5:1, 2:1, 2.5:1, 3:1, 3.5:1, 4:1, 4.5:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1. In some embodiments, the height ratio of the first chamber to the second chamber can range from 1.1:1 to about 50:1, or from about 2.5:1 to about 50:1, or from 2.5 to about 25:1, or from about 2.5:1 to about 15:1. In one embodiment, the height ratio of the first chamber to the second chamber ranges from about 1:1 to about 20:1. In one embodiment, the height ratio of the first chamber to the second chamber ranges from about 1:1 to about 15:1. In one embodiment, the height ratio of the first chamber to the second chamber can be about 10:1.

The height of the first chamber 204 can be of any dimension, e.g., sufficient to accommodate cell height and/or to permit a low shear flow. For example, in some embodiments, the height of the first chamber can range from about 100 μm to about 50 mm, about 200 μm to about 10 mm, about 500 μm to about 5 mm, or about 750 μm to about 2 mm. In one embodiment, the height of the first chamber can be about 150 μm. In one embodiment, the height of the first chamber can be about 1 mm.

The height of the second chamber 206 can be of any dimension provided that the flow rate and/or shear stress of a medium flowing in the second chamber can be maintained within a physiological range, or does not cause any adverse effect to the cells. In some embodiments, the height of the second chamber can range from 20 μm to about 1 mm, or about 50 μm to about 500 μm, or about 75 μm to about 400 μm, or about 100 μm to about 300 μm. In one embodiment, the height of the second chamber can be about 150 μm. In one embodiment, the height of the second chamber can be about 100 μm.

The first chamber and/or the second chamber can have a uniform height along the length of the first chamber and/or the second chamber, respectively. Alternatively, the first chamber and/or the second chamber can each independently have a varying height along the length of the first chamber and/or the second chamber, respectively. For example, a length portion of the first chamber can be substantially taller than the same length portion of the second chamber, while the rest of the first chamber can have a height comparable to or even smaller than the height of the second chamber.

In some embodiments, the first structure and/or second structure of the devices described herein can be further adapted to provide mechanical modulation of the membrane. Mechanical modulation of the membrane can include any movement of the membrane that is parallel to and/or perpendicular to the force/pressure applied to the membrane, including, but are not limited to, stretching, bending, compressing, vibrating, contracting, waving, or any combinations thereof. Different designs and/or approaches to provide mechanical modulation of the membrane between two chambers have been described, e.g., in the International Patent App. Nos. PCT/US2009/050830, and PCT/US2014/071570, the contents of which are incorporated herein by reference in their entireties, and can be adapted herein to modulate the membrane in the devices described herein.

In some embodiments, the devices described herein can be placed in or secured to a cartridge. In accordance with some embodiments of some aspects described herein, the device can be integrated into a cartridge and form a monolithic part. Some examples of a cartridge are described in the International Patent App. No. PCT/US2014/047694, the content of which is incorporated herein by reference in its entirety. The cartridge can be placed into and removed from a cartridge holder that can establish fluidic connections upon or after placement and optionally seal the fluidic connections upon removal. In some embodiments, the cartridge can be incorporated or integrated with at least one sensor, which can be placed in direct or indirect contact with a fluid flowing through a specific portion of the cartridge during operation. In some embodiments, the cartridge can be incorporated or integrated with at least one electric or electronic circuit, for example, in the form of a printed circuit board or flexible circuit. In accordance with some embodiments of some aspects described herein, the cartridge can comprise a gasketing embossment to provide fluidic routing.

In some embodiments, the cartridge and/or the device described herein can comprise a barcode. The barcode can be unique to types and/or status of the cells present on the membrane. Thus, the barcode can be used as an identifier of each device adapted to mimic function of at least a portion of a specific tissue and/or a specific tissue-specific condition. Prior to operation, the barcode of the cartridge can be read by an instrument so that the cartridge can be placed and/or aligned in a cartridge holder for proper fluidic connections and/or proper association of the data obtained during operation of each device. In some embodiments, data obtained from each device include, but are not limited to, cell response, immune cell recruitment, intracellular protein expression, gene expression, cytokine/chemokine expression, cell morphology, functional data such as effectiveness of an endothelium as a barrier, concentration change of an agent that is introduced into the device, or any combinations thereof

In some embodiments, the device can be connected to the cartridge by an interconnect adapter that connects some or all of the inlet and outlet ports of the device to microfluidic channels or ports on the cartridge. Some examples interconnect adapters are disclosed in U.S. Provisional Application No. 61/839,702, filed on Jun. 26, 2013, and the International Patent Application No. PCT/US2014/044417 filed Jun. 26, 2014, the contents of each of which are hereby incorporated by reference in their entirety. The interconnect adapter can include one or more nozzles having fluidic channels that can be received by ports of the device described herein. The interconnect adapter can also include nozzles having fluidic channels that can be received by ports of the cartridge.

In some embodiments, the interconnect adaptor can comprise a septum interconnector that can permit the ports of the device to establish transient fluidic connection during operation, and provide a sealing of the fluidic connections when not in use, thus minimizing contamination of the cells and the device. Some examples of a septum interconnector are described in U.S. Provisional Application No. 61/810,944 filed Apr. 11, 2013, the content of which is incorporated herein by reference in its entirety.

Membrane: The membrane 208 is oriented along a plane 208P parallel to the x-y plane between the first chamber 204 and the second chamber 206. It should be noted that although one membrane 208, more than one membrane 208 can be configured in devices which comprise more than two chambers.

The membrane separating the first chamber and the second chamber in the devices described herein can be porous (e.g., permeable or selectively permeable), non-porous (e.g., non-permeable), rigid, flexible, elastic or any combinations thereof.

Accordingly, the membrane 208 can have a porosity of about 0% to about 99%. As used herein, the term “porosity” is a measure of total void space (e.g., through-holes, openings, interstitial spaces, and/or hollow conduits) in a material, and is a fraction of volume of total voids over the total volume, as a percentage between 0 and 100% (or between 0 and 1). A membrane with substantially zero porosity is non-porous or non-permeable.

As used interchangeably herein, the terms “non-porous” and “non-permeable” refer to a material that does not allow any molecule or substance to pass through.

In some embodiments, the membrane can be porous and thus allow molecules, cells, particulates, chemicals and/or media to migrate or transfer between the first chamber 204 and the second chamber 206 via the membrane 208 from the first chamber 204 to the second chamber 206 or vice versa.

As used herein, the term “porous” generally refers to a material that is permeable or selectively permeable. The term “permeable” as used herein means a material that permits passage of a fluid (e.g., liquid or gas), a molecule, a whole living cell and/or at least a portion of a whole living cell, e.g., for formation of cell-cell contacts. The term “selectively permeable” as used herein refers to a material that permits passage of one or more target group or species, but act as a barrier to non-target groups or species. For example, a selectively-permeable membrane can allow passage of a fluid (e.g., liquid and/or gas), nutrients, wastes, cytokines, and/or chemokines from one side of the membrane to another side of the membrane, but does not allow whole living cells to pass therethrough. In some embodiments, a selectively-permeable membrane can allow certain cell types to pass therethrough but not other cell types.

he permeability of the membrane to individual matter/species can be determined based on a number of factors, including, e.g., material property of the membrane (e.g., pore size, and/or porosity), interaction and/or affinity between the membrane material and individual species/matter, individual species size, concentration gradient of individual species between both sides of the membrane, elasticity of individual species, and/or any combinations thereof.

A porous membrane can have through-holes or pore apertures extending vertically and/or laterally between two surfaces 208A and 208B of the membrane (FIG. 1B), and/or a connected network of pores or void spaces (which can, for example, be openings, interstitial spaces or hollow conduits) throughout its volume. The porous nature of the membrane can be contributed by an inherent physical property of the selected membrane material, and/or introduction of conduits, apertures and/or holes into the membrane material.

In some embodiments, a membrane can be a porous scaffold or a mesh. In some embodiments, the porous scaffold or mesh can be made from at least one extracellular matrix polymer (e.g., but not limited to collagen, alginate, gelatin, fibrin, laminin, hydroxyapatite, hyaluronic acid, fibroin, and/or chitosan), and/or a biopolymer or biocompatible material (e.g., but not limited to, polydimethylsiloxane (PDMS), polyurethane,styrene-ethylene-butylene-styrene (SEBS), poly(hydroxyethylmethacrylate) (pHEMA), polyethylene glycol, polyvinyl alcohol and/or any biocompatible material described herein for fabrication of the device first structure and/or second structure) by any methods known in the art, including, e.g., but not limited to, electrospinning, cryogelation, evaporative casting, and/or 3D printing. See, e.g., Sun et al. (2012) “Direct-Write Assembly of 3D Silk/Hydroxyapatite Scaffolds for Bone Co-Cultures.” Advanced Healthcare Materials, no. 1: 729-735; Shepherd et al. (2011) “3D Microperiodic Hydrogel Scaffolds for Robust Neuronal Cultures.” Advanced Functional Materials 21: 47-54; and Barry III et al. (2009) “Direct-Write Assembly of 3D Hydrogel Scaffolds for Guided Cell Growth.” Advanced Materials 21: 1-4, for examples of a 3D biopolymer scaffold or mesh that can be used as a membrane in the device described herein.

In some embodiments, a membrane can comprise an elastomeric portion fabricated from a styrenic block copolymer-comprising composition, e.g., as described in the International Pat. App. No. PCT/US2014/071611, can be adopted in the devices described herein, the contents of each of which are incorporated herein by reference in its entirety. In some embodiments, the styrenic block copolymer-comprising composition can comprise SEBS and polypropylene.

In some embodiments, a membrane can be a hydrogel or a gel comprising an extracellular matrix polymer, and/or a biopolymer or biocompatible material. In some embodiments, the hydrogel or gel can be embedded with a conduit network, e.g., to promote fluid and/or molecule transport. See, e.g., Wu et al. (2011) “Omnidirectional Printing of 3D Microvascular Networks.” Advanced Materials 23: H178-H183; and Wu et al. (2010) “Direct-write assembly of biomimetic microvascular networks for efficient fluid transport.” Soft Matter 6: 739-742, for example methods of introducing a conduit network into a gel material.

In some embodiments, a porous membrane can be a solid biocompatible material or polymer that is inherently permeable to at least one matter/species (e.g., gas molecules) and/or permits formation of cell-cell contacts. In some embodiments, through-holes or apertures can be introduced into the solid biocompatible material or polymer, e.g., to enhance fluid/molecule transport and/or cell migration. In one embodiment, through-holes or apertures can be cut or etched through the solid biocompatible material such that the through-holes or apertures extend vertically and/or laterally between the two surfaces of the membrane 208A and 208B. It should also be noted that the pores can additionally or alternatively incorporate slits or other shaped apertures along at least a portion of the membrane 208 which allow cells, particulates, chemicals and/or fluids to pass through the membrane 208 from one section of the central channel to the other.

The pores of the membrane (including pore apertures extending through the membrane 208 from the top 208A to bottom 208B surfaces thereof and/or a connected network of void space within the membrane 208) can have a cross-section of any size and/or shape. For example, the pores can have a pentagonal, circular, hexagonal, square, elliptical, oval, diamond, and/or triangular shape.

The cross-section of the pores can have any width dimension provided that they permit desired molecules and/or cells to pass through the membrane. In some embodiments, the pore size of the membrane should be big enough to provide the cells sufficient access to nutrients present in a fluid medium flowing through the first chamber and/or the second chamber. In some embodiments, the pore size can be selected to permit passage of cells (e.g., immune cells and/or cancer cells) from one side of the membrane to the other. In some embodiments, the pore size can be selected to permit passage of nutrient molecules. In some embodiments, the width dimension of the pores can be selected to permit molecules, particulates and/or fluids to pass through the membrane 208 but prevent cells from passing through the membrane 208. In some embodiments, the width dimension of the pores can be selected to permit cells, molecules, particulates and/or fluids to pass through the membrane 208. Thus, the width dimension of the pores can be selected, in part, based on the sizes of the cells, molecules, and/or particulates of interest. In some embodiments, the width dimension of the pores (e.g., diameter of circular pores) can be in the range of 0.01 microns and 20 microns, or in one embodiment, approximately 0.1-15 microns, or approximately 1-10 microns. In one embodiment, the pores have a width of about 7 microns.

In an embodiment, the porous membrane 208 can be designed or surface patterned to include micro and/or nanoscopic patterns therein such as grooves and ridges, whereby any parameter or characteristic of the patterns can be designed to desired sizes, shapes, thicknesses, filling materials, and the like.

The membrane 208 can have any thickness to suit the needs of a target application. In some embodiments, the membrane can be configured to deform in a manner (e.g., stretching, retracting, compressing, twisting and/or waving) that simulates a physiological strain experienced by the cells in its native microenvironment. In these embodiments, a thinner membrane can provide more flexibility. In some embodiments, the membrane can be configured to provide a supporting structure to permit growth of a defined layer of cells thereon. Thus, in some embodiments, a thicker membrane can provide a greater mechanical support. In some embodiments, the thickness of the membrane 208 can range between 70 nanometers and 100 μm, or between 1 μm and 100 μm, or between 10 and 100 μm. In one embodiment, the thickness of the membrane 208 can range between 10 μm and 80 μm. In one embodiment, the thickness of the membrane 208 can range between 30 μm and 80 μm. In one embodiment, the thickness of the membrane 208 can be about 50 μm.

While the membrane 208 generally have a uniform thickness across the entire length or width, in some embodiments, the membrane 208 can be designed to include regions which have lesser or greater thicknesses than other regions in the membrane 208. The decreased thickness area(s) can run along the entire length or width of the membrane 208 or can alternatively be located at only certain locations of the membrane 208. The decreased thickness area can be present along the bottom surface of the membrane 208 (i.e. facing second chamber 206), or additionally/alternatively be on the opposing surface of the membrane 208 (i.e. facing second chamber 204). It should also be noted that at least portions of the membrane 208 can have one or more larger thickness areas relative to the rest of the membrane, and capable of having the same alternatives as the decreased thickness areas described above.

In some embodiments, the membrane can be coated with substances such as various cell adhesion promoting substances or ECM proteins, such as fibronectin, laminin, various collagen types, glycoproteins, vitronectin, elastins, fibrin, proteoglycans, heparin sulfate, chondroitin sulfate, keratin sulfate, hyaluronic acid, fibroin, chitosan, or any combinations thereof. In some embodiments, one or more cell adhesion molecules can be coated on one surface of the membrane 208 whereas another cell adhesion molecule can be applied to the opposing surface of the membrane 208, or both surfaces can be coated with the same cell adhesion molecules. In some embodiments, the ECMs, which can be ECMs produced by cells, such as primary cells or embryonic stem cells, and other compositions of matter are produced in a serum-free environment.

In an embodiment, one can coat the membrane with a cell adhesion factor and/or a positively-charged molecule that are bound to the membrane to improve cell attachment and stabilize cell growth. The positively charged molecule can be selected from the group consisting of polylysine, chitosan, poly(ethyleneimine) or acrylics polymerized from acrylamide or methacrylamide and incorporating positively-charged groups in the form of primary, secondary or tertiary amines, or quaternary salts. The cell adhesion factor can be added to the membrane and is fibronectin, laminin, various collagen types, glycoproteins, vitronectin, elastins, fibrin, proteoglycans, heparin sulfate, chondroitin sulfate, keratin sulfate, hyaluronic acid, tenascin, antibodies, aptamers, or fragments or analogs having a cell binding domain thereof. The positively-charged molecule and/or the cell adhesion factor can be covalently bound to the membrane. In another embodiment, the positively-charged molecule and/or the cell adhesion factor are covalently bound to one another and either the positively-charged molecule or the cell adhesion factor is covalently bound to the membrane. Also, the positively-charged molecule or the cell adhesion factor or both can be provided in the form of a stable coating non-covalently bound to the membrane.

In an embodiment, the cell attachment-promoting substances, matrix-forming formulations, and other compositions of matter are sterilized to prevent unwanted contamination. Sterilization can be accomplished, for example, by ultraviolet light, filtration, gas plasma, ozone, ethylene oxide, and/or heat. Antibiotics can also be added, particularly during incubation, to prevent the growth of bacteria, fungi and other undesired micro-organisms. Such antibiotics include, by way of non-limiting example, gentamicin, streptomycin, penicillin, amphotericin and ciprofloxacin.

In some embodiments, the membrane and/or other components of the devices described herein can be treated using gas plasma, charged particles, ultraviolet light, ozone, or any combinations thereof.

Using the devices described herein, one can study biotransformation, absorption, as well as drug clearance, metabolism, delivery, and toxicity. The activation of xenobiotics can also be studied. The bioavailability and transport of chemical and biological agents across epithelial layers as in a tissue or organ, e.g., gut, lung, and across endothelial layers as in blood vessels, such as for a BBB-on-chip, and across embodiments of gut epithelial layers for drug metabolism can also be studied. The acute basal toxicity, acute local toxicity or acute organ-specific toxicity, teratogenicity, genotoxicity, carcinogenicity, and mutagenicity, of chemical agents can also be studied. Effects of infectious biological agents, biological weapons, harmful chemical agents and chemical weapons can also be detected and studied. Infectious diseases and the efficacy of chemical and biological agents to treat these diseases, as well as optimal dosage ranges for these agents, can be studied. The response of organs in vivo to chemical and biological agents, and the pharmacokinetics and pharmacodynamics of these agents can be detected and studied using the devices described herein. The impact of genetic content on response to the agents can be studied. The amount of protein and gene expression in response to chemical or biological agents can be determined. Changes in metabolism in response to chemical or biological agents can be studied as well using devices described herein.

In some embodiments, the devices described herein (e.g., a Gut-on-Chip) can be used to assess the clearance of a test compound. For clearance studies, the disappearance of a test compound can be measured (e.g. using mass spec) in the media of the top chamber, bottom chamber, or both chambers (divided by a membrane comprising intestinal epithelial cells).

For example, in accordance to one aspect of the invention, a Gut-on-Chip drug-metabolizing performance can be measured by i) disposing a substrate compound with known liver metabolites in the media of the top chamber, bottom chamber, or both chambers; and ii) measuring the amount of generated metabolite in the media of the top chamber, bottom chamber or both chambers (e.g. using mass spec). As is known in the art, the choice of the substrate and measured metabolite can help provide information on specific liver drug-metabolism enzymes (e.g. CYP450 isoforms, Phase II enzymes, etc.)

In some embodiments, the devices described herein (e.g., a Gut-on-Chip) can be used to assess the induction or inhibition potential of a test compound. For induction or inhibition studies a variety of tests are contemplated. For example, induction of CYP3A4 activity in the liver is one of main causes of drug-drug interactions, which is a mechanism to defend against exposure to drugs and toxin, but can also lead to unwanted side-effects (toxicity) or change the efficacy of a drug. A reliable and practical CYP3A induction assay with human hepatocytes in a 96-well format has been reported, where various 96-well plates with different basement membrane were evaluated using prototypical inducers, rifampicin, phenytoin, and carbamazepine. See Drug Metab. Dispo. (2010) November; 38(11):1912-6.

According to one aspect of the invention, the induction or inhibition potential of a test compound at a test concentration can be evaluated by i) disposing the test compound in the media of the top chamber, bottom chamber or both chambers at the test concentration; ii) exposing the device for a selected period of time; and iii) assessing the induction or inhibition of enzymes by comparing performance to a measurement performed before the test compound was applied, to a measurement performed on a Gut-on-Chip that was subjected to a lower concentration of test compound (or no test compound at all), or both. In some embodiments, the performance measurement can comprise an RNA expression level. In some embodiments, the performance measurement comprises assessing drug-metabolizing capacity.

In some embodiments, the devices described herein (e.g., a Gut-on-Chip) can be used to identify in vivo metabolites of a test compound or agent, and optionally the in vivo ratio of these metabolites. According to one aspect of the invention, in vivo metabolites can be identified by i) disposing a test compound or agent in the media of the top chamber, bottom chamber, or both chambers; and ii) measuring the concentration of metabolites in the media of the top chamber, bottom chamber, or both chambers. In some embodiments, the measuring of the concentration of metabolites comprises mass spectroscopy.

In some embodiments, the devices described herein (e.g., a Gut-on-Chip) can be used to identify the toxicity of a test compound or agent at a test concentration. According to one aspect of the invention, toxicity can be evaluated by i) disposing a test compound in the media of the top chamber, bottom chamber, or both chambers; and ii) measuring one or more toxicity endpoints selected from the list of leakage of cellular enzymes (e.g., lactose dehydrogenase, alanine aminotransferase, aspartate aminotransferase) or material (e.g., adenosine triphosphate), variation in RNA expression, inhibition of drug-metabolism capacity, reduction of intracellular ATP (adenosine triphosphate), cell death, apoptosis, and cell membrane degradation.

A. Closed Top Microfluidic Chips Without Gels.

In one embodiment, closed top gut-on-chips do not contain gels, either as a bulk gel or a gel layer. Thus, in one embodiment, the device generally comprises (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the first and second chambers are enclosed. The first side of the membrane may have an extracellular matrix composition disposed thereon, wherein the extracellular matrix (ECM) composition comprises an ECM coating layer. In some embodiments, an ECM gel layer e.g. ECM overlay, is located over the ECM coating layer.

Additional embodiments are described herein that may be incorporated into closed top chips without gels.

B. Closed Top Microfluidic Chips With Gels.

In one embodiment, closed top gut-on-chips do contain gels, such as a gel layer, or bulk gel, including but not limited to a gel matrix, hydrogel, etc. Thus, in one embodiment, the device generally comprises (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the first and second chambers are enclosed. In some embodiments, the device further comprises a gel. In some embodiments, the gel is a continuous layer. In some embodiments, the gel is a layer of approximately the same thickness across the layer. In some embodiments, the gel is a discontinuous layer. In some embodiments, the gel has different thicknesses across the layer. In some embodiments, the first side of the membrane may have a gel layer. In some embodiments, a gel is added to the first side of the membrane without an ECM layer. The first side of the membrane may have an extracellular matrix composition disposed thereon, wherein the extracellular matrix (ECM) composition comprises an ECM coating layer. In some embodiments, an ECM gel layer e.g. ECM overlay, is located over the ECM coating layer. In some embodiments, the gel layer is above the ECM coating layer. In some embodiments, the ECM coating layer may have a gel layer on the bottom, i.e. the side facing the membrane. In some embodiments, the gel overlays the ECM gel layer.

In some embodiments, a gel may be on top of neurons. In some embodiments, a gel may be below neurons.

Additional embodiments are described herein that may be incorporated into closed top chips with gels.

C. Closed Top Microfluidic Chips With Simulated Lumens.

A closed top gut-on-chip comprising a gel-lined simulated lumen may be used for generating a more physiological relevant model of gastrointestinal tissue. In some embodiments, closed top gut-on-chips further comprise a gel simulated three-dimensional (3-D) lumen. In other words, a 3-D lumen may be formed using gels by providing simulated intestinal villi (e.g. viscous fingers) and/or mimicking intestinal folds. In a preferred embodiment, the gel forms a lumen, i.e. by viscous fingering patterning.

Using viscous fingering techniques, e.g. viscous fingering patterning, a simulated intestinal lumen may be formed by numerous simulated intestinal villi structures. Intestinal villi (singular: villus) refer to small, finger-like projections that extend into the lumen of the small intestine. For example, healthy small intestine mucosa contains these small finger-like projections of tissue that are present along the lumen as folds of circular plica finger-like structures. A villus is lined on the luminal side by an epithelia cell layer, where the microvillus of the epithelial cells (enterocytes) faces the lumen (i.e. apical side). Viscous fingers may be long and broad, for mimicking villi in the duodenum of the small intestine, while thinner or shorter viscous fingers may be used for mimicking villi in other parts of the gastrointestinal tract. As one example, viscous fingers may be formed and used to mimic epithelial projections in the colon.

Methods to create three-dimensional (3-D) lumen structures in permeable matrices are known in the art. One example of a 3-D structure forming at least one lumen is referred to as “viscous fingering”. One example of viscous fingering methods that may be used to for form lumens, e.g. patterning lumens, is described by Bischel, et al. “A Practical Method for Patterning Lumens through ECM Hydrogels via Viscous Finger Patterning.” J Lab Autom. 2012 April; 17(2): 96-103. Author manuscript; available in PMC 2012 Jul. 16, herein incorporated by reference in its entirety. In one example of a viscous finger patterning method for use with microfluidic gut-on-chips, lumen structures are patterned with an ECM hydrogel.

“Viscous” generally refers to a substance in between a liquid and a solid, i.e. having a thick consistency. A “viscosity” of a fluid refers to a measure of its resistance to gradual deformation by shear stress or tensile stress. For liquids, it corresponds to an informal concept of “thickness”; for example, honey has a much higher viscosity than water.

“Viscous fingering” refers in general to the formation of patterns in “a morphologically unstable interface between two fluids in a porous medium.

A “viscous finger” generally refers to the extension of one fluid into another fluid. Merely as an example, a flowable gel or partially solidified gel may be forced, by viscous fingering techniques, into another fluid, into another viscous fluid in order to form a viscous finger, i.e. simulated intestinal villus.

In some embodiments, the lumen can be formed by a process comprising (i) providing the first chamber filled with a viscous solution of the first matrix molecules; (ii) flowing at least one or more pressure-driven fluid(s) with low viscosity through the viscous solution to create one or more lumens each extending through the viscous solution; and (iii) gelling, polymerizing, and/or cross linking the viscous solution. Thus, one or a plurality of lumens each extending through the first permeable matrix can be created.

In another embodiment, gel is added to a channel for making a lumen.

In some embodiments as described herein, the first and second permeable matrices can each independently comprise a hydrogel, an extracellular matrix gel, a polymer matrix, a monomer gel that can polymerize, a peptide gel, or a combination of two or more thereof. In one embodiment, the first permeable matrix can comprise an extracellular matrix gel, (e.g. collagen). In one embodiment, the second permeable matrix can comprise an extracellular matrix gel and/or protein mixture gel representing an extracellular microenvironment, (e.g. MATRIGEL®. In some embodiments, the first and second permeable matrixes can each independently comprise a polymer matrix. Methods to create a permeable polymer matrix are known in the art, including, e.g. but not limited to, particle leaching from suspensions in a polymer solution, solvent evaporation from a polymer solution, sold-liquid phase separation, liquid-liquid phase separation, etching of specific “block domains” in block co-polymers, phase separation to block-co-polymers, chemically cross-linked polymer networks with defined permabilities, and a combination of two or more thereof.

Another example for making branched structures using fluids with differing viscosities is described in “Method And System For Integrating Branched Structures In Materials” to Katrycz, Publication number US20160243738, herein incorporated by reference in its entirety.

Regardless of the type of lumen formed by a gel and/or structure, cells can be attached to theses structures either to lumen side of the gel and/or within the gel and/or on the side of the gel opposite the lumen. Thus, three-dimensional (3-D) lumen gel structures may be used in several types of embodiments for closed top microfluidic chips, e.g. epithelial cells can be attached to outside of the gel, or within the gel. In some embodiments, LPDCs may be added within the gel, or below the gel, on the opposite side of the lumen. In some embodiments, stoma cells are added within the gel. In some embodiments, stomal cells are attached to the side of the gel opposite from the lumen. In some embodiments, endothelial cells are located below the gel on the side opposite the lumen. In some embodiments, endothelial cells may be present within the gel.

Additional embodiments are described herein that may be incorporated into closed top chips with simulated 3D lumens containing a gel.

D. Double Membrane Devices (Chips).

In some embodiments, a chip described in U.S. Pat. No. 8,647,861, herein incorporated in its entirety, is used in at least one step for providing innervated gastrointestinal tissue on-chip. In one embodiment, a chip having at least two membranes and at least 3 channels is used for providing neuronal cells.

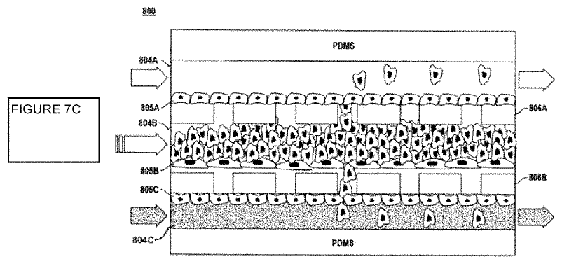

FIG. 7A illustrates a perspective view of an organ mimic device in accordance with an embodiment that contains three parallel microchannels separated by two porous membranes. As shown in FIG. 7A, the organ mimic device 800 includes operating microchannels 802 and an overall central microchannel 804 positioned between the operating microchannels 802. The overall central microchannel 804 includes multiple membranes 806A, 806B positioned along respective parallel x-y planes which separate the microchannel 804 into three distinct central microchannels 804A, 804B and 804C. The membranes 806A and 806B may be porous, elastic, or a combination thereof. Positive and/or negative pressurized media may be applied via operating channels 802 to create a pressure differential to thereby cause the membranes 806A, 806B to expand and contract along their respective planes in parallel.

FIG. 7B illustrates a perspective view of an organ mimic device in accordance with an embodiment. As shown in FIG. 7B, the tissue interface device 900 includes operating microchannels 902A, 902B and a central microchannel 904 positioned between the microchannels 902. The central microchannel 904 includes multiple membranes 906A, 906B positioned along respective parallel x-y planes. Additionally, a wall 910 separates the central microchannel into two distinct central microchannels, having respective sections, whereby the wall 910 along with membranes 904A and 904B define microchannels 904A, 904B, 904C, and 904D. The membranes 906A and 906B at least partially porous, elastic or a combination thereof.

The device in FIG. 7B differs from that in FIG. 7A in that the operating microchannels 902A and 902B are separated by a wall 908, whereby separate pressures applied to the microchannels 902A and 902B cause their respective membranes 904A and 904B to expand or contract. In particular, a positive and/or negative pressure may be applied via operating microchannels 902A to cause the membrane 906A to expand and contract along its plane while a different positive and/or negative pressure is applied via operating microchannels 902B to cause the membrane 906B to expand and contract along its plane at a different frequency and/or magnitude. Of course, one set of operating microchannels may experience the pressure while the other set does not experience a pressure, thereby only causing one membrane to actuate. It should be noted that although two membranes are shown in the devices 800 and 900, more than two membranes are contemplated and can be configured in the devices.

In an example, shown in FIG. 7C, the device containing three channels described in FIG. 7A has two membranes 806A and 806B which are coated to determine cell behavior of a vascularized tumor. In particular, membrane 806A is coated with a lymphatic endothelium on its upper surface 805A and with stromal cells on its lower surface, and stromal cells are also coated on the upper surface of the second porous membrane 805B and a vascular endothelium on its bottom surface 805C. Tumor cells are placed in the central microchannel surrounded on top and bottom by layers of stromal cells on the surfaces of the upper and lower membranes in section 804B. Fluid such as cell culture medium or blood enters the vascular channel in section 804 C. Fluid such as cell culture medium or lymph enters the lymphatic channel in section 804A. This configuration of the device 800 allows researchers to mimic and study tumor growth and invasion into blood and lymphatic vessels during cancer metastasis. In the example, one or more of the membranes 806A, 806B may expand/contract in response to pressure through the operating microchannels. Additionally or alternatively, the membranes may not actuate, but may be porous or have grooves to allow cells to pass through the membranes.

In some embodiments top side is referring to an upper surface of a membrane. In some embodiments, bottom side is referring to a lower surface of a membrane.

### III. Open Top Microfluidic Chips.

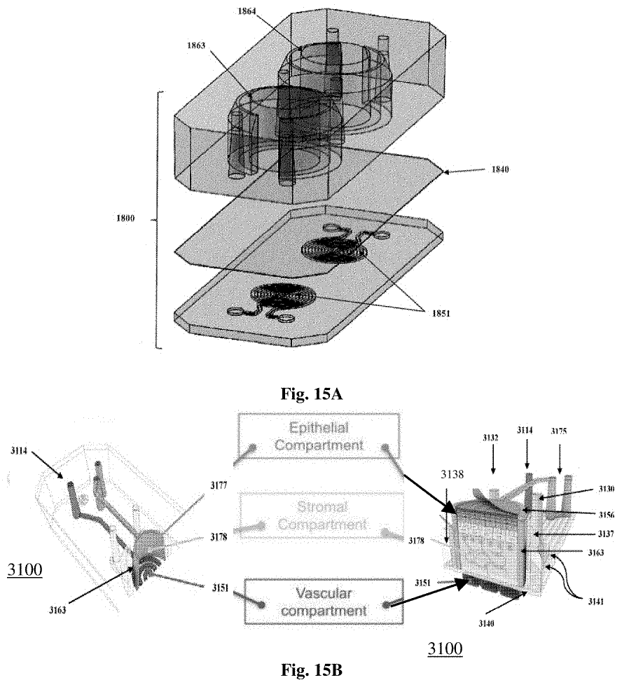

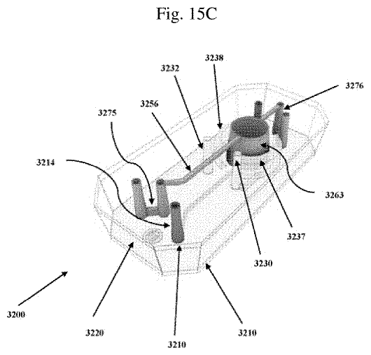

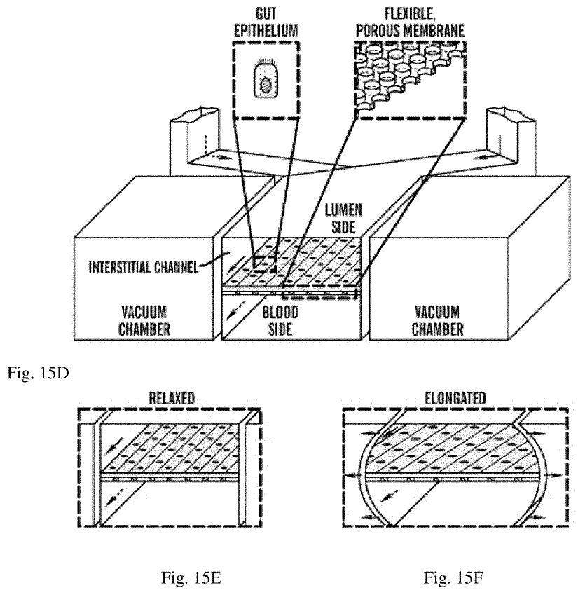

The present disclosure relates to gut-on-chips, such as fluidic devices comprising one or more cells types for the simulation one or more of the function of gastrointestinal tract components. Accordingly, the present disclosure additionally describes open-top gut-on-chips, see, e.g. schematic in FIG. 15A-C. As another example, the use of an open-top chip allows electrical stimulation, e.g. using electrodes, and allows recording electrical measurements in real-time, e.g. recording electrical output of neurons innervating cells, e.g. intestinal epithelial layer.

FIG. 15A shows an exemplary exploded view of one embodiment of an open-top fluidic device 1800, wherein a membrane 1840 resides between the bottom surface of the first chamber 1863 and the second chamber 1864 and the at least two spiral microchannels 1851. Open-top fluidic devices, such as microfluidic chips, include but are not limited to chips having removable covers, such as removable plastic covers, paraffin covers, tape covers, etc.

In some embodiments of a microfluidic device, it is desirable to include a cover that comprises sensors or actuators. For example, a cover can comprise one or more electrodes that can be used for measurement of electrical excitation. In some embodiments, such as where the device comprises a membrane (e.g., membrane 540), the one or more electrodes can be used to perform a measurement of trans-epithelial electrical resistance (TEER) for the membrane. It may also be desirable to include one or more electrodes on the opposite side of the membrane 540. In some embodiments, the electrodes can be included in a bottom structure (e.g., bottom structure 525). In some embodiments, the bottom structure can be an open bottom with bottom electrodes included on a bottom cover that can be brought into contact with the bottom structure. The bottom cover may support any of the features or variations discussed herein in the context of a top cover, including, for example, removability, fluidic channels, multiple layers, clamping features, etc.

FIG. 15B shows exemplary schematic views of one embodiment of an open-top chip device in relation to exemplary cell compartments, e.g. epithelial, stromal and vascular. In one embodiment, the present invention contemplates a stretchable open top chip device 3100 comprising a chamber 3163 comprising an epithelial region 3177 and a dermal region 3178. In one embodiment, the epithelial region comprises an epithelial cell layer. In one embodiment, the dermal region comprises a dermal cell layer, wherein said epithelial cell layer adheres to the surface of the dermal cell layer. In one embodiment, the device further comprises a spiral microchannel 3151 in fluid communication with a fluid inlet port 3114, wherein the microchannel comprises a plurality of vascular cells, in one embodiment, a membrane 3140 is placed between the chamber dermal cell layer and the microchannel plurality of vascular cells. In one embodiment, the device further comprises an upper microchannel with a circular chamber 3156 connected to a fluid or gas port pair 3175. In one embodiment, the device further comprises a first vacuum port 3130 connected to a first vacuum chamber 3137 and a second vacuum port 3132 connected to a second vacuum chamber 3138. In one embodiment, the membrane 3140 comprises a PDMS membrane comprising a plurality of pores 3141, wherein said pores 3141 are approximately 50 μm thick, approximately 7 urn in diameter, packed as 40 Um hexagons, wherein each pore has a surface area of approximately 0.32 cm². Although it is not necessary to understand the mechanism of an invention, it is believed that the pore surface area contacts a gel layer (if present). FIGS. 15A and 15B.

FIG. 15C shows another exemplary schematic of an open top microfluidic chip showing embodiments of a stretchable open top chip device 3200. In one embodiment, the present invention contemplates a stretchable open top chip device 3200 comprising: i) a fluidic cover 3210 comprising an upper microchannel with a circular chamber 3256 configured with a first fluid or gas port pair 3275 and second fluid or gas port pair 3276; a fluid inlet port 3214, a fluid outlet port 3216, a first vacuum port 3230 and a second vacuum port 3232; ii) a top structure 3220 comprising a chamber 3263, a first vacuum chamber 3237 connected to the first vacuum port 3230, and a second vacuum chamber 3238, connected to the second vacuum port 3232, wherein the upper microchannel with a circular chamber 3256 seals with the top surface of the chamber 3263; and iii) a bottom structure 3225 layered underneath said top structure 3220. FIG. 15C.

Many of the problems associated with earlier systems can be solved by providing an open-top style microfluidic device that allows topical access to one or more parts of the device or cells that it comprises. For example, the microfluidic device can include a removable cover, that when removed, provides access to the cells of interest in the microfluidic device. In some aspects, the microfluidic devices include systems that constrain fluids, cells, or biological components to desired area(s). The improved systems provide for more versatile experimentation when using microfluidic devices, including improved application of treatments being tested, improved seeding of additional cells, and/or improved aerosol delivery for select tissue types.

It is also desirable in some aspects to provide access to regions of a cell-culture device. For example, it can be desirable to provide topical access to cells to (i) apply topical treatments with liquid, gaseous, solid, semi-solid, or aerosolized reagents, (ii) obtain samples and biopsies, or (iii) add additional cells or biological/chemical components

Therefore, the present disclosure relates to fluidic systems that include a fluidic device, such as a microfluidic device with an opening that provides direct access to device regions or components (e.g. access to the gel region, access to one or more cellular components, etc.). Although the present disclosure provides an embodiment wherein the opening is at the top of the device (referred to herein with the term “open top”), the present invention contemplates other embodiments where the opening is in another position on the device. For example, in one embodiment, the opening is on the bottom of the device. In another embodiment, the opening is on one or more of the sides of the device. In another embodiment, there is a combination of openings (e.g. top and sides, top and bottom, bottom and side, etc.).

While detailed discussion of the “open top” embodiment is provided herein, those of ordinary skill in the art will appreciate that many aspects of the “open top” embodiment apply similarly to open bottom embodiments, as well as open side embodiments or embodiments with openings in any other regions or directions, or combinations thereof. Similarly, the device need not remain “open” throughout its use; rather, as several embodiments described herein illustrate, the device may further comprise a cover or seal, which may be affixed reversibly or irreversibly. For example, removal of a removable cover creates an opening, while placement of the cover back on the device closes the device. The opening, and in particular the opening at the top, provides a number of advantages, for example, allowing (i) the creation of one or more gel layers for simulating the application of topical treatments on the cells, tissues, or organs, or (ii) the addition of chemical or biological components such as the seeding of additional cell types for simulated tissue and organ systems. The present disclosure further relates to improvement in fluidic system(s) that improve the delivery of aerosols to simulated tissue and organ systems, such as simulated gastrointestinal tissues.

The present invention contemplates a variety of uses for these open top microfluidic devices and methods described herein. In one embodiment, the present invention contemplates a method of topically testing an agent (whether a drug, food, gas, or other substance) comprising 1) providing a) an agent and b) microfluidic device comprising i) a chamber, said chamber comprising a lumen and projections into the lumen, said lumen comprising ii) a gel matrix anchored by said projections and comprising cell in, on or under said gel matrix, said gel matrix positioned above iii) a porous membrane and under iv) a removable cover, said membrane in contact with v) fluidic channels; 2) removing said removable cover; and 3) topically contacting said cells in, on or under said gel matrix with said agent. In one embodiment, said agent is in an aerosol. In one embodiment, agent is in a liquid, gas, gel, semi-solid, solid, or particulate form. These uses may apply to the open top microfluidic chips described below and herein.

A. Open Top Microfluidic Chips Without Gels.

In one embodiment, open top gut-on-chips do not contain gels, either as a bulk gel or a gel layer. Thus, the present invention also contemplates, in one embodiment, a layered structure comprising i) fluidic channels covered by ii) a porous membrane, said membrane comprising iii) a layer of cells and said membrane positioned below said cells. In one embodiment, there is a removable cover over the cells.

Additional embodiments are described herein that may be incorporated into open top chips without gels.

B. Open Top Microfluidic Chips with Gels.

Furthermore, the present disclosure contemplates improvements to fluidic systems that include a fluidic device, such as a microfluidic device with an open-top region that reduces the impact of stress that can cause the delamination of tissue or related component(s) (e.g., such as a gel layer). Thus, in a preferred embodiment, the open-top microfluidic device comprises a gel matrix. In one embodiment, the open-top microfluidic device does not contain a bulk gel.

In one embodiment, said gel matrix is patterned (or at least a portion of it is patterned). In one embodiment, the device further comprises electrodes, e.g. electrodes are configured for measuring the electrophysiology of said brain microvascular endothelial cells. In one embodiment, said gel matrix comprises collagen. It is not intended that the present invention be limited to embodiments with only one gel or gel layer. In one embodiment, the layered structure further comprises a second gel matrix (e.g. positioned under said membrane). The gel(s) or coatings can be patterned or not patterned. Moreover, when patterned, the pattern need not extend to the entire surface. For example, in one embodiment, at least a portion of said gel matrix is patterned.

The present invention also contemplates, in one embodiment, a layered structure comprising i) fluidic channels covered by ii) a porous membrane, said membrane comprising iii) a layer of cells and said membrane positioned below iv) a gel matrix. In one embodiment, there is a removable cover over the gel matrix (and/or cells). It is not intended that the present invention be limited to embodiments with only one gel or gel layer. In one embodiment, the layered structure further comprises a second gel matrix (e.g. positioned under said membrane). The gel(s) or coatings can be patterned or not patterned. Moreover, when patterned, the pattern need not extend to the entire surface. For example, in one embodiment, at least a portion of said gel matrix is patterned. It is not intended that the present invention be limited by the nature or components of the gel matrix or gel coating. In one embodiment, gel matrix comprises collagen. A variety of thickness is contemplated. In one embodiment of the layered structure, said gel matrix is between 0.2 and 6 mm in thickness.

Also described is a simulated lumen further comprising gel projections into the simulated lumen. Thus, in yet another embodiment, the present invention contemplates a microfluidic device comprising i) a chamber, said chamber comprising a lumen and projections in the lumen, said lumen comprising ii) a gel matrix anchored by said projections, said gel matrix positioned above iii) a porous membrane, said membrane in contact with iv) fluidic channels. In one embodiment, said membrane comprises cells. The projections serve as anchors for the gel. The projections, in one embodiment, project outward from the sidewalls. The projections, in another embodiment, project upward. The projects, in another embodiment, project downward. The projections can take a number of forms (e.g. a T structure, a Y structure, a structure with straight or curving edges, etc.). In some embodiments, there are two or more projections; in other embodiments, there are four or more projections to anchor the gel matrix. In one embodiment, the membrane is above said fluidic channels.

In other embodiments, open top microfluidic chips comprise partial lumens as described herein for closed top chips. Thus, in some embodiments, open top microfluidic chips comprise lumens formed by viscous fingering described herein for closed top chips.

Lumen gel structures may be used in several types of embodiments for open top microfluidic chips, e.g. epithelial cells or parenchymal cells can be attached to outside of the gel, or within the gel. In some embodiments, LPDCs may be added within the gel, below the gel, or above the gel. In some embodiments, stomal cells are added within the gel. In some embodiments, stomal cells are attached to the side of the gel opposite from the lumen. In some embodiments, endothelial cells are located below the gel on the side opposite the lumen. In some embodiments, endothelial cells may be present within the gel.

IV. Fluidic Devices with Structural Anchors.

In one embodiment, the present invention contemplates a fluidic device comprising an open-top cavity with structural anchors on the vertical wall surfaces that serve to at least in part, to prevent gel shrinkage-induced delamination, a porous membrane (optionally stretchable) positioned in the middle over a microfluidic channel(s). See, for nonlimiting examples, WO2017096297, Open-top microfluidic device with structural anchors, published 8 Jun. 2017.

In one embodiment, the present invention contemplates a method of topically testing an agent (whether a drug, food, gas, or other substance) comprising 1) providing a) an agent and b) microfluidic device comprising i) a chamber, said chamber comprising a lumen and projections into the lumen, said lumen comprising ii) a gel matrix anchored by said projections and comprising cell in, on or under said gel matrix, said gel matrix positioned above iii) a porous membrane and under iv) a removable cover, said membrane comprising brain microvascular endothelial cells in contact with v) fluidic channels; 2) removing said removable cover; and 3) topically contacting said cells in, on or under said gel matrix with said agent. In one embodiment, said agent is in an aerosol. In one embodiment, agent is in a liquid, gas, gel, semisolid, solid, or particulate form.

In one embodiment, the present invention contemplates a fluidic cover comprising a fluidic channel, said fluidic cover configured to engage a microfluidic device. In one embodiment, the microfluidic device comprises an open chamber, and wherein said fluidic cover configured to cover and close said open chamber. In one embodiment, the fluidic cover further comprises one or more electrodes.

In one embodiment, the present invention contemplates an assembly comprising a fluidic cover comprising a fluidic channel, said fluidic cover detachably engaged with a microfluidic device. In one embodiment, the microfluidic device comprises an open chamber, and wherein said fluidic cover configured to cover and close said open chamber. In one embodiment, the open chamber comprises a non-linear lumen. In one embodiment, the non-linear lumen is circular. In one embodiment, the fluidic cover further comprises one or more electrodes.

In some embodiments of a microfluidic device, it is desirable to include a cover that comprises sensors or actuators. For example, a cover can comprise one or more electrodes that can be used for measurement of electrical excitation. In some embodiments, such as where the device comprises a membrane (e.g., membrane 208, 806A, 806B, 904A, 904B, 540), the one or more electrodes can be used to perform a measurement of trans-epithelial electrical resistance (TEER) for the membrane. It may also be desirable to include one or more electrodes on the opposite side of the membrane 540. In some embodiments, the electrodes can be included in a bottom structure. In some embodiments, the bottom structure can be an open bottom with bottom electrodes included on a bottom cover that can be brought into contact with the bottom structure. The bottom cover may support any of the features or variations discussed herein in the context of a top cover, including, for example, removability, fluidic channels, multiple layers, clamping features, etc.

Additional embodiments are described herein that may be incorporated into open top chips with gels, or without gels.

The present invention also contemplates, in one embodiment, a layered structure comprising i) fluidic channels covered by ii) a porous membrane, said membrane comprising iii) a layer of cells and said membrane positioned below iv) a gel matrix. In one embodiment, there is a removable cover over the gel matrix (and/or cells). While not intending to be limited to any particular cell type, in one embodiment, the cells are brain microvascular endothelial cells. In one embodiment of the layered structure, it further comprises neurons on, in or under the gel matrix. In still another embodiment of the layered structure, it further comprises astrocytes on, in or under the gel matrix. Cells can be positioned in various different places (in or on the layered structure). In one embodiment, the layer of brain microvascular endothelial cells is positioned on the bottom of the membrane so as to be in contact with the fluidic channels. It is not intended that the present invention be limited to any particular source of cells. In one embodiment, the brain microvascular endothelial cells are primary cells.

FIG. 15D shows another exemplary schematic of a fluidic gut-on-a-Chip wherein a body has a central microchannel therein; and an at least partially porous and at least partially flexible porous membrane positioned within the central microchannel and along a plane. The membrane is configured to separate the central microchannel to form a first central microchannel having a lumen side and a second central microchannel having a blood side, wherein a first fluid is applied through the first central microchannel and a second fluid is applied through the second central microchannel. There is at least one operating channel (vacuum chambers) separated from the first and second central microchannels by a first microchannel wall. The membrane is mounted to the first microchannel wall, and when a pressure is applied to the operating channel (one or more vacuum chambers), it can cause the membrane to expand or contract along the plane within the first and the second central microchannels. In some embodiments, one side of the membrane, example lumen side, can be seeded with epithelial cells to mimic an epithelial layer while another side of the membrane, example blood side, can be seeded with microvascular endothelial cells to mimic capillary vessels.

FIGS. 15E and 15F schematically depicts relaxed and elongated cell layers, via a stretched membrane, in an exemplary gut On-Chip. See, WO2013086486, Integrated Human Organ-On-Chip Microphysiological Systems. Publication Date: Jun. 13, 2013, herein incorporated by reference in its entirety.

### V. Embedded Electrodes.

To make measurements, electrodes can be included in the layered structure, i.e. electrodes configured for measuring the electrophysiology of cells, such as neuronal cells, brain microvascular endothelial cells. Other cells can also be tested (e.g. muscle cells). In some embodiments, electrodes are integrated into fluidic devices, see, WO2017106727, herein incorporated by reference in its entirety.

The present invention also contemplates, in one embodiment, a method of testing, comprising 1) providing a layered structure comprising i) fluidic channels covered by ii) a porous membrane, said membrane comprising iii) a layer of cells in contact with said fluidic channels, said membrane positioned below iv) a gel matrix, said gel matrix under a removable cover; and 2) measuring the electrophysiology of said cells. A variety of cell types can be tested. In one embodiment, the cells are brain microvascular endothelial cells. In another embodiment, the cells are muscle cells. In one embodiment of this method, the layered structure further comprises v) electrodes configured for measuring the electrophysiology of said brain microvascular endothelial cells. In one embodiment, said measuring comprises TEER measurements with said electrodes. In one embodiment, said TEER measurements indicate tight cell-to-cell junctions between said brain microvascular endothelial cells. In another embodiment, said measuring of step 2) comprises patch clamp measurements, extracellular electrophysiology measurements, imaging using calcium-sensitive dyes or proteins, or imaging using voltage-sensitive dyes or proteins. In one embodiment, said brain microvascular endothelial cells express the marker Glut 1. In one embodiment of this method, said layered structure further comprises neurons on, in or under said gel matrix. In one embodiment, the layered structure further comprises a second gel matrix positioned under said membrane.

In one embodiment, a fluidic device comprising electrodes, such as a trans-epithelial electrical resistance (TEER)-Chip, is used for testing barrier function. In one embodiment, a TEER-Chip is used for providing a real-time readout. In one embodiment, a TEER-Chip has trans-endothelial electrical resistance (TEER) electrodes. Integration of in-line TEER measurements on chip allows continuous interrogation of the barrier function integrity of tissues. For example, abrupt changes in TEER measurements can be observed during inflammatory challenges to endothelial cells (FIG. 7). We have integrated sensors into prototype Organ-Chips so that these measurements can be made in real-time throughout an experiment.

For TEER measurements, an embodiment is contemplated wherein a layered structure or microfluidic device 2300 has a top electrode 2371 and a bottom electrode 2372 configured for measuring the electrophysiology of said brain microvascular endothelial cells.

FIG. 16 shows one embodiment for microfluidic devices as contemplated herein that is configured for electrophysiological measurements (e.g., for example, patch clamp measurements using transepithelial electric resistance (TEER). In one embodiment, the top electrode 2371 is a chromium/gold (Cr—Au) electrode. In one embodiment, the bottom electrode 2372 is a chromium/gold (Cr—Au) electrode.

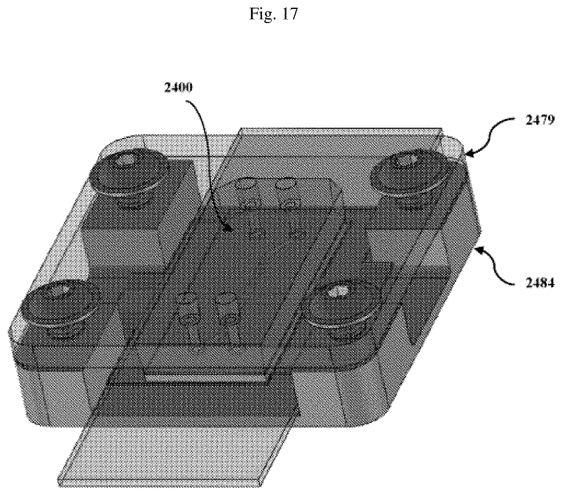

FIG. 17 shows one embodiment for microfluidic devices as contemplated herein that is configured for electrophysiological measurements (e.g., for example, patch clamp measurements using transepithelial electric resistance (TEER). However, it is not intended that the present invention be limited to only TEER measurements. In one embodiment, the present invention contemplates a method of testing, comprising 1) providing a layered TEER microfluidic device 2300 comprising i) a bottom structure 2325 comprising at least one upper microfluidic channel 2334 covered by ii) a porous membrane 2340, said membrane comprising iii) a layer of brain microvascular endothelial cells in contact with said at least one upper microfluidic channel, said membrane position below iv) a gel matrix (or other porous volume), said gel matrix (preferably) under a removable cover; and 2) measuring the electrophysiology of said brain microvascular endothelial cells. In one embodiment, the porous membrane 2340 is covered by a top structure 2320. In one embodiment, the layered TEER microfluidic device 2300 further comprises a top clamp 2379 and a bottom clamp 2384, wherein said top clamp 2379 has at least one access hole 2381. In one embodiment, the at least one access hole 2381 is configured to align with a port adapter 2383. In some embodiments, a glass slide 2382 is placed between the bottom electrode 2372 and the bottom clamp 2384. In one embodiment, the top clamp 2379 comprises a lasercut acrylic material. In one embodiment, the port adapter 2383 comprises a cast PDMS material. In one embodiment, the top electrode 2371 comprises a lasercut PET material. In one embodiment, the bottom electrode 2372 comprises a lasercut PET material. In one embodiment, the top structure 2320 comprises an open-top channel gasket having a cast PDMS material. In one embodiment, the bottom structure 2325 comprises an open-bottom channel gasket having a spin-coated and laser-cut PDMS material. In one embodiment, the bottom clamp 2384 comprises a 3D printed ABS plastic material. Although not limiting, the top clamp 2379 and bottom clamp 2384 may be attached with M4 screws 2386 and M4 nuts 2387. Although it is not necessary to understand the mechanism of an invention, it is believed that a TEER microfluidic device is clamped because the various layered components described above would be difficult to glue (e.g., bonding). It is further believed that a clamp facilitates an ability to open the device and have direct access to cells for patch-clamp measurements. Alternatively, if this openable feature is not desired, the device layers can be bonded together. A fully assembled layered TEER chip 2400 between a top clamp 479 and bottom clamp 2384 is presented in FIG. 17.

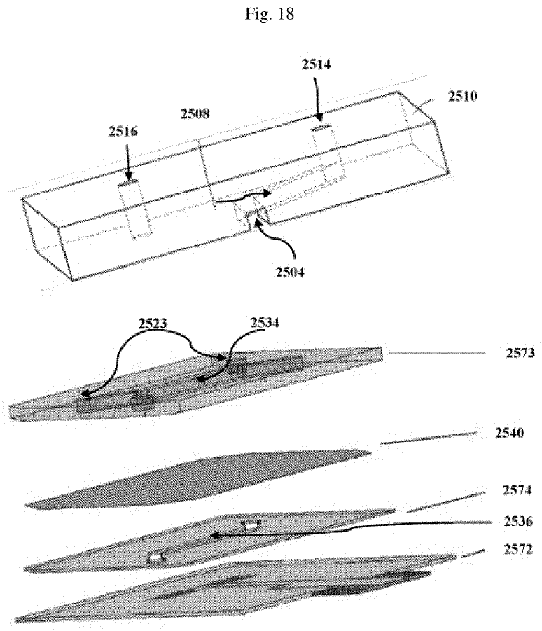

FIG. 18 shows one embodiment for microfluidic devices as contemplated herein that is configured for electrophysiological measurements (e.g., for example, patch clamp measurements using transepithelial electric resistance (TEER). A variety of techniques are contemplated including but not limited to using a multi-electrode array or patch clamping. In one embodiment, the present invention contemplates an “open top” design that allows for patch clamping through the opening. For example, an open-top patch clamp layered TEER microfluidic device 2500 may comprise an optional top microfluidic cover 2510 comprising an open region 2504, an optional top microfluidic cover fluidic channel 2508 and inlet port 2514, wherein the open region 2504 provides access to an open-top channel gasket 2573. In one embodiment, the TEER microfluidic subassembly device 2500 comprises an open-top channel gasket 2573 having at least one upper microchannel 2534 in fluid communication with at least one upper microchannel well 2523. A porous membrane 2540 is placed between the open-top channel gasket 2573 and an open-bottom channel gasket 2574, wherein the open-bottom channel gasket 2574 comprises at least one lower microchannel 2536. A bottom electrode 2572 is placed underneath the open-top channel gasket/porous membrane/open-bottom channel gasket layered stack. In one embodiment, the bottom electrode 2572 is a chromium/gold electrode.

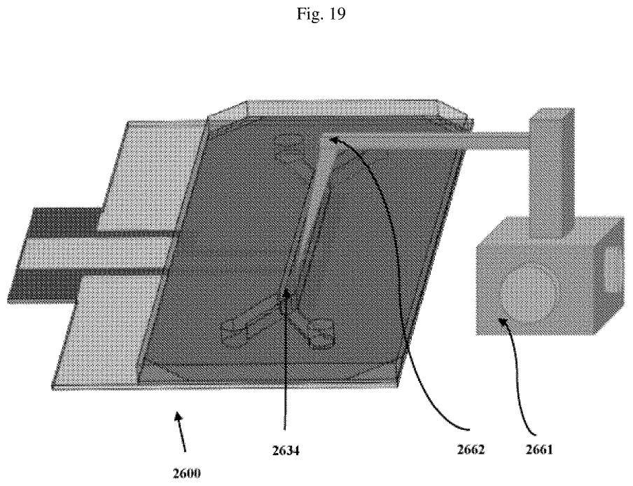

FIG. 19 shows one embodiment for microfluidic devices as contemplated herein that is configured for electrophysiological measurements (e.g., for example, patch clamp measurements using transepithelial electric resistance (TEER). See, for examples without a specific description of innervated tissues, WO2017143049, Improved Blood-Brain Barrier Endothelial Cells Derived From Pluripotent Stem Cells For Blood-Brain Barrier Models, Publication Date: Aug. 24, 2017. An open-top TEER microfluidic subassembly patch clamp device 2600 may be exposed to allow access with a micro-manipulator 2661. FIG. 19. For example, a micromanipulator arm 2661 may be placed directly within an upper microchannel 2634. Although it is not necessary to understand the mechanism of an invention, it is believed that the micromanipulator arm 2661 may, for example, add reagents, remove a fluid sample, add cells and/or remove cells. This allows the configuration of the patch clamp device 2600 to interchangeably go between a flow configuration (e.g., where the upper microchannel 2634 is not exposed) and an open configuration (e.g., where the upper microchannel 2634 is exposed).

### VI. Chip Activation.

A. Chip Activation Compounds.

In one embodiment, bifunctional crosslinkers are used to attach one or more extracellular matrix (ECM) proteins. A variety of such crosslinkers are available commercially, including (but not limited to) the following compounds:

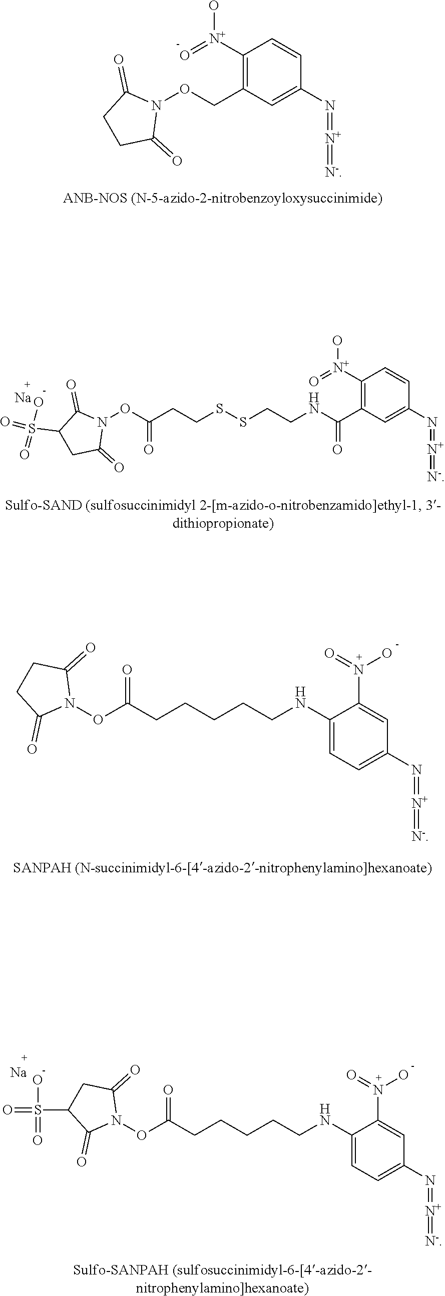

By way of example, sulfosuccinimidyl 6-(4′-azido-2′-nitrophenyl-amino) hexanoate or “Sulfo-SANPAH” (commercially available from Pierce) is a long-arm (18.2 angstrom) crosslinker that contains an amine-reactive N-hydroxysuccinimide (NHS) ester and a photoactivatable nitrophenyl azide. NHS esters react efficiently with primary amino groups (—NH₂) in pH 7-9 buffers to form stable amide bonds. The reaction results in the release of N-hydroxy-succinimide. When exposed to UV light, nitrophenyl azides form a nitrene group that can initiate addition reactions with double bonds, insertion into C—H and N—H sites, or subsequent ring expansion to react with a nucleophile (e.g., primary amines). The latter reaction path dominates when primary amines are present.

Sulfo-SANPAH should be used with non-amine-containing buffers at pH 7-9 such as 20 mM sodium phosphate, 0.15M NaCl; 20 mM HEPES; 100 mM carbonate/bicarbonate; or 50 mM borate. Tris, glycine or sulfhydryl-containing buffers should not be used. Tris and glycine will compete with the intended reaction and thiols can reduce the azido group.

For photolysis, one should use a UV lamp that irradiates at 300-460 nm. High wattage lamps are more effective and require shorter exposure times than low wattage lamps. UV lamps that emit light at 254 nm should be avoided; this wavelength causes proteins to photodestruct. Filters that remove light at wavelengths below 300 nm are ideal. Using a second filter that removes wavelengths above 370 nm could be beneficial but is not essential.

B. Exemplary methods of Chip Activation.

Prepare and Sanitize Hood-Working Space

1. S-1 Chip Handling—Use aseptic technique, hold Chip using Carrier
a. Use 70% ethanol spray and wipe the exterior of Chip package prior to bringing into hood
b. Open package inside hood
c. Remove Chip and place in sterile petri dish (6 Chips/Dish)
d. Label Chips and Dish with respective condition and Lot #
   2. Surface Activation with Chip Activation Compound (light and time sensitive)
a. Turn off light in biosafety hood
b. Allow vial of Chip Activation Compound powder to fully equilibrate to ambient temperature (to prevent condensation inside the storage container, as reagent is moisture sensitive)
c. Reconstitute the Chip Activation Compound powder with ER-2 solution
   i. Add 10 ml Buffer, such as HEPES, into a 15 ml conical covered with foil
   ii. Take 1 ml Buffer from above conical and add to chip Activation Compound (5 mg) bottle, pipette up and down to mix thoroughly and transfer to same conical
   iii. Repeat 3-5 times until chip Activation Compound is fully mixed
   iv. NOTE: Chip Activation Compound is single use only, discard immediately after finishing Chip activation, solution cannot be reused
d. Wash channels
   i. Inject 200 ul of 70% ethanol into each channel and aspirate to remove all fluid from both channels
   ii. Inject 200 μl of Cell Culture Grade Water into each channel and aspirate to remove all fluid from both channels
   iii. Inject 200 μl of Buffer into each channel and aspirate to remove fluid from both channels
e. Inject Chip Activation Compound Solution (in buffer) in both channels
   i. Use a P200 and pipette 200 μl to inject Chip Activation Compound/Buffer into each channel of each chip (200 μl should fill about 3 Chips (Both Channels))
   ii. Inspect channels by eye to be sure no bubbles are present. If bubbles are present, flush channel with Chip Activation Compound/Buffer until bubbles have been removed
f. UV light activation of Chip Activation Compound Place Chips into UV light box
   i. UV light treat Chips for 20 min

While the Chips are being treated, prepare ECM Solution.
ii. After UV treatment, gently aspirate Chip Activation Compound/Buffer from channels via same ports until channels are free of solution
iii. Carefully wash with 200 μl of Buffer solution through both channels and aspirate to remove all fluid from both channels
iv. Carefully wash with 200 μl of sterile DPBS through both channels
v. Carefully aspirate PBS from channels and move on to: ECM-to-Chip.

References incorporated by reference in their entirety include: Corpening et al., Dev Dyn. 2008, 237(4): 1119-1132 and Lee et al., Nat Prot, 2010 March; Vol 5: 688-701.

### EXPERIMENTAL

Exemplary protocol for Neural Crest differentiation includes (Chambers et al., Meth in Mol Biol. 2013, 1307: 329-343), herein incorporated by reference in its entirety. In brief, mouse embryonic fibroblast-conditioned media (MEF-CM); and KSR Gibco™ KnockOut™ Serum Replacement (KnockOut™ SR) are used comprising at specific feedings, different concentrations of media and factors, see, FIG. 5. This differentiation method provided, passage 1, Day 11 a population of cells comprising HNK1+ plus p75+ cells for use in Example A for providing glial cells and neurons.

### Example A—Innervation Using iNeural Crest Cell Derived Neurons

In one embodiment, glial cells and neuron cells may find use for innervation on chips.

Glial cells (e.g. S100B+) and neuron cells (e.g. TUJ1+) were induced from HNK1+/p75+ sorted passage 1-Day 11 (P1d11) neural crest cell populations differentiated from PS cells (e.g. 20,000 cells/cm²).

In one embodiment, beads were used for isolating (sorting out) HNK1+ plus p75+ cells. HNK1+ plus p75+ cells were then seeded onto a second membrane (lower) of a two-membrane chip. In one embodiment, Human Colonic Epithelial Cells (NCM460) were seeded on top of the upper (first membrane). In one embodiment, HNK1+ plus p75+ cells were seeded on top of Human Colonic Microvascular Epithelial Cells (cHIMECs). In one embodiment, cHIMECs are a source of NGF. In another embodiment, HNK1+ plus p75+ cells were seeded on top of Human Intestinal Smooth Muscle Cells (SMCs). In one embodiment, SMCs are a source of GDNF.

After 6 days of culture under flow with a Flow rate: 30 ul/hr, NCM460/cHIMECs and NCM460/SMCs showed S100β+ (glial cells) and TUJ1+ (neurons).

In one embodiment, S100β+ TUJ1+ (neurons) may be added to a chip for inducing innervation.

### Example B—Barrier Function—Electric Resistance

There are many ways to evaluate the integrity and physiology of an in vitro system that mimics a physiological barrier, e.g. an epithelial layer, an endothelial layer, a parenchymal cell barrier, etc. Two common methods are Transepithelial Electric Resistance (TEER) and dye particle diffusion measurements, e.g. Lucifer Yellow (LY) dye particle rejection, Luciferase Yellow, 450 Daltons (Da), Cascade blue particles, 3 kDa, etc., travel across cell monolayers. Because dye particle movement is based upon passive paracellular diffusion (through spaces between cells), dye particles have low permeability, i.e. movement, through intact cell layer barriers. Therefore, dye particles are considerably impeded in passing across cell monolayers with tight junctions. Permeability (Papp) for LY of <5 to 12 nm/s was reported to be indicative of a well-established monolayer having intact barrier function.

TEER measures the resistance to pass current across one or more cell layers on a membrane. Specifically, this electrical resistance is a direct measurement of the resistance of the cell monolayer to the transport of ions. The measurement may be affected by the pore size and density of the membrane, but it aims to ascertain cell and/or tissue properties. The TEER value is considered a good measure of the integrity of a cell monolayer.

Thus, in one embodiment, barrier function is monitored, including but not limited to innervated tissue on-chip.

As one example, in one embodiment of a BBB-chip, such as described in Example B, barrier function was measured by flowing a fluorescent molecule on one side of the chip then measuring the amount that crosses the endothelial cell layer (FIG. 23B). The resolution of this measurement is limited by the time it takes for a sufficient sample of fluorescent molecules to flow through the endothelial cell layer of the chip.

FIG. 23A-C shows exemplary Real-time TEER measurements in and exemplary BBB-chip. FIG. 23A (left) shows a gradual increase in barrier function upon initiation of flow and then an abrupt drop after TNF-a challenge. The barrier function of the same BBB-chip measured by flowing a fluorescent molecule on one side of the chip and measuring how much of the molecules crosses the endothelial cells, FIG. 23B (middle). The resolution of this measurement is limited by the time it takes for a sufficient sample to flow through the chip. A model of an exemplary TEER-Chip design FIG. 23C (right) utilizing electrodes oriented in a manner that allows electrical resistance of the cells on the membrane to be measured in real-time.

### Example C—Stimulating Neurons for Electrical Measurements

In one embodiment, a TEER-Chip comprising electrodes is used for testing compounds for inhibiting neuron function, i.e. Ca++ ion movement across membranes, electrical signaling, etc. In one embodiment, a TEER-Chip comprising electrodes is used for testing compounds for inducing neuronal function by electrically stimulating innervated neurons using attached electrodes, embedded electrodes, etc. In one embodiment, a TEER-Chip comprising electrodes is used for testing compounds for inducing neuronal function by electrically stimulating innervated neurons for inducing muscle twitch. Exemplary embodiments of attached electrodes are shown in FIGS. 16-20 and described herein.

In one embodiment, neurons are stimulated while directly stimulating the organ cells. In one embodiment, neurons are stimulated without directly stimulating the organ cells. Further, at least one of the advantages of using a fluidic device comprising a membrane, such that when directly stimulating cells on one side of a membrane, the membrane may prevent direct electrical stimulation of cells on the other side of the membrane. For example, electrodes may be attached to the neuronal compartment (or channel containing neuronal cells), such that the electrical stimulation predominantly does not get to the other side of the membrane or channel. As another example, applying a chemical/agent/drug/soluble factor to neuronal side in a way that predominantly does not make it to the other side due to the characteristics of the membrane.

Alternatively, in some embodiments, electrical connections to neurons are provided in open top microfluidic chips. This, in some embodiments, an innervated open top microfluidic chip allows for electrical stimulation of neurons. In some embodiments, an innervated open top microfluidic chip allows for recording neuronal electrical emissions.

### Example D—Inhibiting Neurons and/or Muscle Twitch

In one embodiment, a TEER-Chip comprising electrodes is used for testing compounds for inhibiting neuron function, i.e. Ca++ ion movement across membranes, electrical signaling, etc. In one embodiment, a TEER-Chip comprising electrodes is used for testing compounds for inhibiting muscle twitch.

Another nonlimiting example of a tool for real-time analysis of cellular function is integrated microscopy. This allows for real-time readouts of cell morphology, cell motility, transport of fluorescent molecules across the tissue barrier, changes in function of reporter cell lines, Calcium ions in neuronal circuits, etc. Exemplary embodiments of attached electrodes are shown in FIGS. 16-20 and described herein.

### Example E—Interactions of Epithelial Mucosa with Sensory Neurons and Microbiome

Previous studies showed a correlation between decreasing immune functions in humans with an increasing virulence of bacteria. Thus, in some embodiments, a human innervated Intestine-Chip (hiIC) is contemplated for use with the addition of immune cells and pathogenic bacteria. In some embodiments, the immune cell-innervated Intestine-Chip (hiIC) is used to test the immune response of this model with and without, or before and after, the addition of a pathogenic microorganism, such as a known pathogenic bacteria strain. In some embodiments, biological read outs include but are not limited to assessing the activation of the incorporated primary sensory neurons as an in vivo-relevant biological read out, disrupted barrier function, etc., for identifying bacteria-mediated disruption of the intestinal response (or defense) to pathogens. Such an understanding of the disruption of regulation of barrier function, etc., may be used in testing known and novel therapeutic targets for use in treatments for autoimmune associated microbial responses and infectious disorders of the intestine.

### Example F—Chemical Capsaicin Stimulation of Neurons On-Chip

In one embodiment, a chemical may be used to stimulate innervated neurons on-chip. Merely as an example, Capsaicin, e.g. (trans-8-methyl-N-vanillyl-6-nonenamide) may be used to provide a model for inducing a pain stimulus in neurons on-chip. Thus, a capsaicin (pain) induced model using human iPS-derived sensory neurons (Nociceptors) that “sense” pain with clinically relevant readouts, e.g. endpoints, (such as elevated calcium levels, Substance P secretion, etc. was developed as described herein.

Thus, in one embodiment, human iPS-derived sensory neurons (Nociceptors) were differentiated on-chip in the apical channel as described herein. In particular, the apical channel was coated with Laminin 10 μg/ml, then incubated at 4° C. overnight. Unattached laminin was washed away with PBS, then culture medium was flowed in to cover the coated membrane to replace the PBS. Seeded iPS cells were not embedded in a gel.

In one embodiment, endothelial cells were seeded into the basal channel, e.g. Human Dermal Microvascular Endothelial Cells (HMVEC-D), i.e. Primary Dermal Microvascular Endothelial Cells; Normal, Human, Neonatal (HDMVECn) (e.g. ATCC® PCS-110-010™). However, it is not meant to limit the source of HMVEC, such that Primary Dermal Microvascular Endothelial Cells may be obtained from neonatal biopsies, e.g. foreskins. Prior to seeding, basal channels were coated with a thin rat collagen coating. Rat collagen was diluted in distillated water to obtain a final protein concentration at 150 μg/mL−1. The basal channel was coated by incubating the channel in collagen solution at 4° C. overnight. Unattached rat collagen was washed away with PBS, then culture medium was flowed in to cover the coated membrane to replace the PBS.

After seeding iPS cells and endothelial cells, microfluidic chips were incubated under conditions for generating differentiated human iPS-derived sensory neurons (Nociceptors), as described herein.

Such differentiated co-cultures of sensory neurons on-chip maintained their characteristics after 7 days in culture under flow, apical channel flow rate at 30 μl/h for 7 days and simultaneously, endothelial cells in the basal channel were incubated under flow at 60 μl/h for 7 days. See, FIG. 28A-C.

Irritation (as pain simulation) to sensory neurons on-chip was induced by capsaicin (1 μM in DMEM/F12 with 10% FBS, supplemented with BDNF, GDNF, NGF, NT-3 (25 ng/ml each) and Ascorbic Acid (200 μM)) in order to recapitulate the sensation of burning (heat) felt by humans exposed to capsaicin.

Nociceptors responded to capsaicin, as shown by live imaging of calcium using Fluo-4 AM, see FIGS. 28C and 28D.

Fluo-4 AM refers to a calcium sensitive fluorescent dye, loaded onto the sensory neurons. Intracellular calcium fluctuations were monitored over time using high-speed imaging. Acquisition protocols, for the example shown herein, consisted of 5-minute time-lapse sequences of Fluo-4 fluorescence. Alterations in fluorescence as a function of time were measured at a single wavelength (Fluo-4). Analysis and processing, as well as playback of the image sequences for visual inspection, was made using ImageJ/FIJI software. To visualize the spatial and temporal changes in, the raw sequences were processed to highlight changes in fluorescence intensity between frames. Regions of interest over the field of view were selected, and the mean pixel intensity at each frame was measured. The data was first plotted as fluorescence intensity versus and subsequently converted to a relative scale (ΔF/F baseline)

FIG. 28A-D shows exemplary sensory neurons, i.e. Nociceptors, in an apical channel on-chip, maintaining their neuronal characteristics on-chip after 7 days in culture.

FIG. 28A shows a fluorescent image of the entire neuronal channel showing Tuj-1+ neurons (green) upper panel; TRPV1 neurons (red) middle panel; and merged images showing yellow co-staining of Nociceptors, lower panel.

FIG. 28B shows exemplary Nociceptors as yellow co-stained Tuj-1+/TRPV1+ neurons, left panel, Tuj-1+ (green) upper right; TRPV1+ (red) lower right. Scale bars=50 μm.

FIG. 28C shows exemplary Nociceptors stained with Fluo4-AM after irritation by Capsaicin (1 μM), see circled activated nodes.

FIG. 28D shows an exemplary graph of Nociceptors responding to capsaicin, as shown by live imaging of calcium, change in fluorescence (ΔF) compared to fluorescence before stimulation (F).

Chemical stimulation of Nociceptors with Capsaicin (e.g. 1 μM), perfused for 3 hours, was followed by effluent collected from the apical channel (neuronal). This Capsaicin treatment elicited the release of substance P, a pain neurotransmitter. Thus demonstrating the ability of an innervated chip to “Sense” with clinically-relevant endpoints, e.g. calcium flux, above, and release of substance P, see, FIGS. 23A-B.

FIG. 29A-B shows an exemplary read-out of substance P produced by chronic stimulation of Nociceptors (sensory neurons) though perfusion of the neuronal channel with capsaicin.

FIG. 29A shows an exemplary schematic of a closed channel chip comprising nociceptors in the apical channel perfused with capsaicin (1 uM) through the inlet, under flow, where the effluent was collected from the outlet of the apical channel after 3 hours (t=3 h).

FIG. 29B shows an exemplary graph of Substance P (pg/ml) measured in the apical channel effluent, as described in FIG. 23A showing a significant increase in Substance P released after capsaicin.

This model was further developed, as described herein, successfully simulating the process of sensitization that leads to pathological pain by the addition of inflammatory mediators to endothelial media perfused through the vascular channel. As one example, 5 μM each of Prostaglandin E2, bradykinin, serotonin and histamine in Vascular Cell Basal Medium (ATCC® PCS-100-030™) with Microvascular Endothelial Cell Growth Kit-VEGF (ATCC® PCS-110-041™) was perfused through the vascular channel. In other words, this model is intended to simulate peripheral sensitization of Nociceptors. Capsaicin (200 nM) and inflammatory mediators (each 5 μM) were perfused for 3 hours, and the effluents were collected from the apical channel (neuronal). See, FIG. 30A for an exemplary schematic diagram.

As shown below, sensitization of sensory neurons with capsaicin (e.g. 200 nM) resulted in significant increased release of substance P along with a significant increase in vascular permeability, see FIG. 30B and FIG. 31C.

FIG. 30A-B shows an exemplary read-out of substance P produced by chronic stimulation of Nociceptors (sensory neurons) though perfusion of the neuronal channel with capsaicin in the presence of inflammatory mediators simultaneously perfused through the basal channel.

FIG. 30A shows an exemplary schematic of a closed channel chip comprising nociceptors in the apical channel perfused with capsaicin (200 nM in media) through the inlet, under flow. At the same time, inflammatory mediators, i.e. Prostaglandin E2, bradykinin, serotonin and histamine (each 5 μM in media), were added to the inlet of the basal channel. The effluent was collected from the outlet of the apical channel after 3 hours (t=3 h).

FIG. 30B shows an exemplary graph of Substance P (pg/ml) measured in the apical channel effluent, as described in FIG. 29A showing a significant increase in Substance P released after capsaicin treatment in the presence of inflammatory mediators.

Thus, Nociceptors become sensitized by inflammatory mediators and disperse considerably more substance P in the presence of low levels of Capsaicin.

Therefore, another readout of “pain” stimulated sensory neurons is measuring the effect of Nociceptors (e.g. results of producing substance P, see above) on vascular permeability. Thus, in one embodiment, vascular permeability is indicated by VE-cadherin staining of endothelial cells in the basal (vascular) channel. See, FIG. 31A.

FIG. 31A-B shows an exemplary read-out of changes in vascular permeability produced by chronic stimulation of Nociceptors (sensory neurons) though perfusion of the neuronal channel with capsaicin in the presence of inflammatory mediators simultaneously perfused through the basal channel.

FIG. 31A shows exemplary florescent microscope images of endothelial cells in the basal channel. Left, Hoechst staining of nuclei (blue), middle, e-cadherin staining (pink) and right, merged florescent images showing individual cells (blue nuclei) surrounded by e-cadherin.

Vascular endothelial (VE)-cadherin refers to a major adhesion molecule in endothelial cells. Similar to other classical cadherins, the cytoplasmic tail of VE-cadherin associates with various intracellular proteins including β-catenin and p120 catenin. Furthermore, the connection between adherens junctions and actin filaments mediated by VE-cadherin is believed to be crucial for the regulation of blood vascular endothelial functions, including cellular reactions to various endothelial permeability factors and angiogenic growth factors. For further information see, Dejana, et al., “The control of vascular integrity by endothelial cell junctions: Molecular basis and pathological implications.” Dev Cell 16:209-221, 2009; and Vestweber, “VE-cadherin: The major endothelial adhesion molecule controlling cellular junctions and blood vessel formation.” Arterioscler Thromb Vasc Biol; 28:223-232, 2008.

Another readout is obtained by using Dextran (3 kDa) permeability, where Dextran is added to the basal channel then after 24 hours, effluent from the apical channel was collected for measuring the amount of Dextran that diffused through the endothelial barrier into the neuronal channel. See, FIG. 31B.

FIG. 31B shows an exemplary schematic of a vertical chip cross-section of a closed channel chip comprising Nociceptors in the apical channel separated from HMVEC-D by a membrane (yellow) where Dextran (3 kDa—blue dots) added to the basal channel leaked into the apical channel.

Activation of Nociceptors, in the simultaneous presence of Caspacian and inflammatory mediators triggered the local release of neuropeptides, such as substance P, as described above. Further, the simultaneous presence of Caspacian and inflammatory mediator induced a significant increase in vascular permeability with stimulation of vasodilation (neurogenic inflammation) on-chip. See, FIG. 31C.

FIG. 31C shows an exemplary graphical comparison of leakage (percent) of 3 kDa Dextrin into the apical channel between controls, no treatment (left), inflammatory mediators (middle), demonstrating a significant amount of leakage after perfusion with capsaicin (200 nM in media) in the apical channel simultaneously with perfusion of inflammatory mediators, i.e. Prostaglandin E2, Bradykinin, serotonin and histamine (each 5 μM in media), added to the inlet of the basal channel, right, after 3 hours (t=3 h).

All references cited herein are incorporated by reference in their entirety as though fully set forth. Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described.

1-11. (canceled)

12. A method of culturing cells, comprising: a) providing a microfluidic device comprising a membrane, said membrane comprising a first side and a second side; b) seeding a population of induced pluripotent stem cells (iPSC)-derived cortical neurons on said first side and brain endothelial cells on said second side so as to create seeded cells, wherein said cortical neurons comprise a mixture of glutamatergic neurons and GABAergic neurons; c) exposing at least some of said seeded cells to a flow of culture fluid for a period of time; and d) culturing said seeded cells under conditions such that said endothelial cells foam a cell barrier.

13. The method of claim 12, further comprising a step of seeding said first side of said membrane with additional cells, said additional cell comprising astrocyte cells and pericyte cells.

14. The method of claim 12, wherein said a mixture of glutamatergic neurons and GABAergic neurons comprises approximately 0.8 million cells per milliliter of seeding solution.

15. The method of claim 12, wherein said glutamatergic neurons range from 60-90% of said cortical neurons.

16. The method of claim 12, wherein said cortical neurons comprises approximately 80% vesicular glutamate transporter 1 (VGLUT1)+ cells.

17. The method of claim 12, wherein said GABAergic neurons range from 30-40% of said cortical neurons.

18. The method of claim 12, wherein said cortical neurons comprise approximately 30-40% vesicular GABA transporter (VGAT)+cells.

19. The method of claim 12, wherein said cortical neurons comprises neurons having terminal bulbs in contact with endothelial cells.

20. The method of claim 13, wherein said human iPS-derived cortical neurons are in direct contact with astrocytes and pericytes.

21. The method of claim 20, further comprising seeding said first side of said membrane with microglial cells.

22. The method of claim 12, wherein said endothelial cells are human iPSC-derived brain microvascular endothelial cells.

23. The method of claim 12, wherein said cell barrier exhibits a level of leakage of small molecules from the second side to the first side of said membrane.

24. The method of claim 23, wherein said leakage level is less than the level of leakage of a cell barrier in a microfluidic device seeded with brain endothelial cells on said second side and either glutamatergic neurons or GABAergic neurons alone on said first side.

25. The method of claim 13, further comprising the step of inducing an inflammatory condition in at least one cell type.

26. The method of claim 25, wherein said inflamed cell type comprises astrocytes.

27. The method of claim 25, wherein said inflammatory condition is induced by flowing a solution comprising TNF-α into said bottom channel.

28. The method of claim 25, wherein said inflammatory condition increases cell barrier permeability.

29. A method of culturing cells, comprising: a) providing a microfluidic device comprising a membrane, said membrane comprising a first side and a second side; b) seeding a population of neural crest cells derived from induced pluripotent stem cells (iPSC) on said first side and brain endothelial cells on said second side so as to create seeded cells; c) exposing at least some of said seeded cells to a flow of culture fluid for a period of time; and d) culturing said seeded cells under conditions such that at least a portion of said neural crest cells differentiate into a population of cortical neurons.

30. The method of claim 29, wherein said population of neural crest cells comprise HNK1+/p75+ double positive cells.

31. The method of claim 29, wherein said cortical neurons comprise a mixture of glutamatergic neurons and GABAergic neurons.
